(19) United States
(12) Patent Application Publication
(10) Pub. No.: US 2022/0372580 A1
(43) Pub. Date: Nov. 24, 2022

(54) MACHINE LEARNING TECHNIQUES FOR ESTIMATING TUMOR CELL EXPRESSION IN COMPLEX TUMOR TISSUE

(71) Applicant: BostonGene Corporation, Waltham, MA (US)

(72) Inventors: Aleksandr Zaitsev, Drozhzhino (RU); Alexander Bagaev, Moscow (RU); Maksim Chelushkin, Moscow (RU); Valentina Beliaeva, Moscow (RU); Boris Shpak, Krasnoyarsk (RU); Daniiar Dyikanov, Moscow (RU); Anastasia Zotova, Moscow (RU); Michael F. Goldberg, Brookline, MA (US); Cagdas Tazearslan, Waltham, MA (US)

(73) Assignee: BostonGene Corporation, Waltham, MA (US)

(21) Appl. No.: 17/733,941

(22) Filed: Apr. 29, 2022

Related U.S. Application Data
(60) Provisional application No. 63/239,895, filed on Sep. 1, 2021.
(60) Provisional application No. 63/181,365, filed on Apr. 29, 2021.

(51) Int. Cl. C12Q 1/6886; G16B 40/20; G16H 70/20; G16H 20/40; G16H 50/20

(52) U.S. Cl. CPC C12Q 1/6886; G16B 40/20; G16H 70/20; G16H 20/40; G16H 50/20

(57) ABSTRACT

Techniques for using machine learning to estimate tumor expression levels of genes in tumor cells. The techniques include obtaining expression data for a set of genes comprising a first plurality of genes associated with the tumor cells and a second plurality of genes associated with tumor microenvironment cells; determining the tumor expression levels of the first plurality of genes in the tumor cells using a plurality of machine learning models, the determining comprising: generating a first set of features for the first gene; providing the first set of features as input to the first machine learning model to obtain an output comprising a tumor microenvironment expression level estimate of the first gene in the tumor microenvironment cells; and determining a first tumor expression level for the first gene in the tumor cells using the output of the first machine learning model and a total expression level for the first gene.

### RELATED APPLICATIONS

This application claims benefit under 35 U.S.C. § 119(e) of the filing date of U.S. provisional patent application Ser. No. 63/239,895, filed Sep. 1, 2021, entitled “MACHINE LEARNING TECHNIQUES FOR ESTIMATING MALIGNANT CELL GENE EXPRESSION IN COMPLEX TUMOR TISSUE,” Attorney Docket No. B1462.70026US01, and U.S. provisional patent application Ser. No. 63/181,365, filed Apr. 29, 2021, entitled “COMPUTATIONAL MACHINE LEARNING TOOL TO DECIPHER MALIGNANT CELL GENE EXPRESSION FROM COMPLEX TUMOR TISSUE”, Attorney Docket No. B1462.70026US00, the entire contents of each of which are incorporated by reference herein.

### BACKGROUND

In general, complex tumor tissue (or other diseased tissue) may comprise a population of tumor cells and a tumor microenvironment (TIME) which may include, for example, immune cells, fibroblasts, and extracellular matrix proteins.

### SUMMARY

Some embodiments provide for a method for using machine learning to estimate tumor expression levels of genes in tumor cells in a biological sample of a subject having cancer, the biological sample comprising the tumor cells and tumor microenvironment (TME) cells, the method comprising: obtaining expression data for a set of genes, the set of genes comprising a first plurality of genes associated with the tumor cells and a second plurality of genes associated with the tumor microenvironment cells, the expression data comprising first total expression levels for genes in the first plurality of genes and second total expression levels for genes in the second plurality of genes; determining the tumor expression levels of the first plurality of genes in the tumor cells using a plurality of machine learning models, the plurality of machine learning models comprising a respective machine learning model for each gene in the first plurality of genes including a first machine learning model for a first gene in the first plurality of genes, the tumor expression levels including a first tumor expression level for the first gene in the tumor cells, the determining comprising: generating a first set of features for the first gene, the generating including: obtaining, using the expression data, an initial expression level estimate of the first gene in the tumor cells of the biological sample and including the initial expression level estimate of the first gene in the first set of features; including at least some of the first total expression levels in the first set of features; and including at least some of the second total expression levels in the first set of features; providing the first set of features as input to the first machine learning model to obtain an output indicative of a TME expression level estimate of the first gene in the TME cells; and determining the first tumor expression level for the first gene in the tumor cells using the output of the first machine learning model and a total expression level, in the first total expression levels, for the first gene; and outputting the tumor expression levels of the first plurality of genes in the tumor cells.

Some embodiments provide for a system, comprising: at least one processor; at least one non-transitory computer-readable storage medium storing processor-executable instructions that, when executed by the at least one processor, cause the at least one processor to perform a method for using machine learning to estimate tumor expression levels of genes in tumor cells in a biological sample of a subject having cancer, the biological sample comprising the tumor cells and tumor microenvironment (TME) cells, the method comprising: obtaining expression data for a set of genes, the set of genes comprising a first plurality of genes associated with the tumor cells and a second plurality of genes associated with the TME cells, the expression data comprising first total expression levels for genes in the first plurality of genes and second total expression levels for genes in the second plurality of genes; determining the tumor expression levels of the first plurality of genes in the tumor cells using a plurality of machine learning models, the plurality of machine learning models comprising a respective machine learning model for each gene in the first plurality of genes including a first machine learning model for a first gene in the first plurality of genes, the tumor expression levels including a first tumor expression level for the first gene in the tumor cells, the determining comprising: generating a first set of features for the first gene, the generating including: obtaining, using the expression data, an initial expression level estimate of the first gene in the tumor cells of the biological sample and including the initial expression level estimate of the first gene in the first set of features; including at least some of the first total expression levels in the first set of features; and including at least some of the second total expression levels in the first set of features; providing the first set of features as input to the first machine learning model to obtain an output indicative of a TME expression level estimate of the first gene in the TME cells; and determining the first tumor expression level for the first gene in the tumor cells using the output of the first machine learning model and a total expression level, in the first total expression levels, for the first gene; and outputting the tumor expression levels of the first plurality of genes in the tumor cells.

Some embodiments provide for at least one non-transitory computer-readable storage medium storing processor-executable instructions that, when executed by at least one computer hardware processor, cause the at least one computer hardware processor to perform a method for using machine learning to estimate tumor expression levels of genes in tumor cells in a biological sample of a subject having cancer, the biological sample comprising the tumor cells and tumor microenvironment (TME) cells, the method comprising: obtaining expression data for a set of genes, the set of genes comprising a first plurality of genes associated with the tumor cells and a second plurality of genes associated with the TME cells, the expression data comprising first total expression levels for genes in the first plurality of genes and second total expression levels for genes in the second plurality of genes; determining the tumor expression levels of the first plurality of genes in the tumor cells using a plurality of machine learning models, the plurality of machine learning models comprising a respective machine learning model for each gene in the first plurality of genes including a first machine learning model for a first gene in the first plurality of genes, the tumor expression levels including a first tumor expression level for the first gene in the tumor cells, the determining comprising: generating a first set of features for the first gene, the generating including: obtaining, using the expression data, an initial expression level estimate of the first gene in the tumor cells of the biological sample and including the initial expression level estimate of the first gene in the first set of features; including at least some of the first total expression levels in the first set of features; and including at least some of the second total expression levels in the first set of features; providing the first set of features as input to the first machine learning model to obtain an output indicative of a TME expression level estimate of the first gene in the TME cells; and determining the first tumor expression level for the first gene in the tumor cells using the output of the first machine learning model and a total expression level, in the first total expression levels, for the first gene; and outputting the tumor expression levels of the first plurality of genes in the tumor cells.

In some embodiments, the plurality of machine learning models includes a second machine learning model for a second gene in the first plurality of genes and the tumor expression levels include a second tumor expression level for the second gene in the tumor cells, wherein the second machine learning model is different from the first machine learning model and wherein the second gene is different from the first gene. In some embodiments, determining the tumor expression levels of the first plurality of genes in the tumor cells further comprises: generating a second set of features for the second gene; providing the second set of features as input to the second machine learning model to obtain an output indicative of a TME expression level estimate of the second gene in the TME cells; and determining the second tumor expression level for the second gene in the tumor cells using the output of the second machine learning model and a total expression level, in the first total expression levels, for the second gene.

In some embodiments, generating the second set of features for the second gene comprises: obtaining, using the expression data, an initial expression level estimate of the second gene in the tumor cells of the biological sample and including the initial expression level estimate of the second gene in the second set of features; including at least some of the first total expression levels in the second set of features; and including at least some of the second total expression levels in the second set of features.

In some embodiments, the plurality of machine learning models includes a third machine learning model for a third gene in the first plurality of genes and the tumor expression levels include a third tumor expression level for the third gene in the tumor cells, wherein the third machine learning model is different from the first machine learning model and from the second machine learning model, wherein the third gene is different from the second gene and from the first gene. In some embodiments, determining the tumor expression levels of the first plurality of genes in the tumor cells further comprises: generating a third set of features for the third gene; providing the third set of features as input to the third machine learning model to obtain an output comprising a TME expression level estimate of the third gene in the TME cells; and determining the third tumor expression level for the third gene in the tumor cells using the output of the third machine learning model and a total expression level, in the first total expression levels, for the third gene.

In some embodiments, generating the first set of features for the first gene further comprises: obtaining, using the expression data, a first plurality of RNA percentages for a respective plurality of types of cells that occur in the TME, wherein each of the first plurality of RNA percentages indicates a percent of RNA associated with the first gene and originating from cells of a respective type in the TME in the biological sample.

In some embodiments, generating the first set of features for the first gene further comprises including at least some of the first plurality of RNA percentages in the first set of features.

In some embodiments, obtaining the first plurality of RNA percentages comprises processing at least some of the expression data using at least one non-linear regression model.

In some embodiments, the TME cells comprise TME cells of a first type and TME cells of a second type. In some embodiments, the at least some of the expression data includes a first subset of the expression data and a second subset of the expression data. In some embodiments, the at least one non-linear regression model includes a first non-linear regression model and a second non-linear regression model different from the first non-linear regression model. In some embodiments, obtaining the first plurality of RNA percentages comprises: processing the first subset of the expression data using the first non-linear regression model to obtain a first RNA percentage for the TME cells of the first type; and processing the second subset of the expression data using the second non-linear regression model to obtain a second RNA percentage for the TME cells of the second type.

In some embodiments, the first type and the second type are each selected from the group consisting of B cells, CD4+ T cells, CD8+ T cells, endothelial cells, fibroblasts, lymphocytes, macrophages, monocytes, NK cells, and neutrophils, wherein the first type is different from the second type.

In some embodiments, obtaining the initial expression level estimate of the first gene in the tumor cells of the biological sample comprises: obtaining an average TME expression level of the first gene for each of the plurality of types of cells that occur in the TME; determining a weighted sum of the obtained expression levels based on the first plurality of RNA percentages; and subtracting the weighted sum from the total expression level for the first gene to obtain the initial expression level estimate.

Some embodiments further comprise obtaining, using the expression data, a first RNA percentage for the tumor cells, wherein the first RNA percentage indicates a percent of RNA associated with the first gene and originating from the tumor cell of the biological sample.

In some embodiments, determining the first tumor expression level for the first gene in the tumor cells further comprises: subtracting the TME expression level estimate from the total expression level for the first gene; and dividing a result of the subtracting by the first RNA percentage.

In some embodiments, the expression data has been previously obtained at least in part by sequencing the biological sample of the subject having cancer.

In some embodiments, the at least some of the first total expression levels included in the first set of features include total expression levels for at least 25 genes in the first plurality of genes associated with the tumor cells. In some embodiments, the plurality of machine learning models comprises at least 25 machine learning models corresponding to the at least 25 genes.

In some embodiments, each machine learning model of the at least 25 machine learning models comprises a different gradient boost model.

In some embodiments, the at least some of the first total expression levels included in the first set of features include total expression levels for at least 10 genes selected from genes listed in Table 1. In some embodiments, the at least some of the first total expression levels included in the first set of features include total expression levels for at least 25 genes selected from genes listed in Table 1. In some embodiments, the at least some of the first total expression levels included in the first set of features include total expression levels for at least 50 genes selected from genes listed in Table 1. In some embodiments, the at least some of the first total expression levels included in the first set of features include total expression levels for at least 75 genes selected from genes listed in Table 1.

In some embodiments, the first machine learning model of the plurality of machine learning models is a gradient boosted model.

Some embodiments further comprise training the first machine learning by: obtaining training data comprising simulated expression data for genes in the set of genes, wherein the training data is associated with one or more biological samples; generating, using the training data, a training set of features for the first gene; training the first machine learning model to estimate a TME expression level of the first gene, the training comprising: providing the training set of features as input to the first machine learning model to obtain an output comprising an estimate of the TME expression level of the first gene in the TME cells of the one or more biological samples; and updating parameters of the first machine learning model using the estimate of the TME expression level.

In some embodiments, generating the training set of features for the first gene comprises: obtaining, using the simulated expression data, an initial expression level estimate of the first gene in tumor cells of the one or more biological samples and including the initial expression level estimate in the training set of features; and including at least some of the simulated expression levels in the training set of features.

In some embodiments, the first machine learning model was trained at least in part by generating training data comprising simulated expression data, wherein generating the training data comprises: obtaining training expression data for each of one or more biological samples, the training expression data comprising first training expression levels for the first plurality of genes and second training expression levels for the second plurality of genes; generating first simulated expression data using the first training expression levels; generating second simulated expression data using the second training expression levels; and combining the first simulated expression data and the second simulated expression data to produce at least part of the simulated expression data.

Some embodiments further comprise identifying at least one anti-cancer therapy for the subject based on the first tumor expression level for the first gene in the tumor cells.

Some embodiments further comprise administering the at least one anti-cancer therapy.

In some embodiments, the at least one anti-cancer therapy is selected from the group of therapies for the first gene listed in Table 3.

In some embodiments, identifying the at least one anti-cancer therapy for the subject comprises: determining whether the first tumor expression level satisfies at least one criterion associated with the first gene; and after determining that the first tumor expression level satisfies the at least one criterion, selecting the at least one anti-cancer therapy from the group of therapies listed for the first gene in Table 3.

### BRIEF DESCRIPTION OF DRAWINGS

FIG. 1 is a diagram depicting an illustrative technique 100 for estimating tumor expression levels of genes in tumor cells in a biological sample, according to some embodiments of the technology described herein.

FIG. 2A is a flowchart depicting a process 200 for estimating tumor expression levels of genes in tumor cells in a biological sample using machine learning, according to some embodiments of the technology described herein.

FIG. 2B is a flowchart depicting a process 220 for determining a tumor expression level of a gene in the tumor cells of the biological sample using machine learning, according to some embodiments of the technology described herein.

FIG. 2C is a flowchart depicting a process 250 for generating a set of features for a particular gene to be provided as input to a trained machine learning model trained to estimate a tumor microenvironment (TME) expression level of the particular gene, according to some embodiments of the technology described herein.

FIG. 3A is a diagram of an illustrative technique for estimating tumor expression levels of genes expressed in tumor cells of a biological sample, according to some embodiments of the technology described herein.

FIG. 3B is a diagram depicting an illustrative example of sets of features generated for the genes expressed in tumor cells of the biological sample, according to some embodiments of the technology described herein.

FIG. 4 is a block diagram of an example system 400 for estimating tumor expression levels of genes in tumor cells in a biological sample, according to some embodiments of the technology described herein.

FIG. 5A and FIG. 5B depict illustrative examples for estimating a tumor expression level of a gene in tumor cells of a biological sample, according to some embodiments of the technology described herein.

FIG. 6 is a flowchart depicting a process 600 for training a machine learning model to estimate a tumor microenvironment (TME) expression level of a gene in TME cells of a biological sample, according to some embodiments of the technology described herein.

FIG. 7A and FIG. 7B are diagrams depicting an exemplary technique for generating training data for training various machine learning models described herein, the process including generating simulated expression data as part of the training data, according to some embodiments of the technology described herein.

FIG. 8A is a flowchart depicting an exemplary process 800 for determining RNA percentages based on expression data, according to some embodiments of the technology described herein.

FIG. 8B is a flowchart illustrating an example implementation of process 800 for determining RNA percentages based on expression data, according to some embodiments of the technology described herein.

FIG. 8C is a flowchart illustrating an example implementation of act 816a of method 800, according to some of the embodiments of the technology described herein.

FIG. 9 is a diagram depicting example techniques for preparing data for training, validating, and testing a machine learning model for estimating TME expression levels of genes in TME cells of one or more biological samples, according to some embodiments of the technology described herein.

FIG. 10 shows graphs depicting results showing effectiveness of the techniques described herein for estimating tumor cell expression on an artificial transcriptomes dataset, according to some embodiments of the technology described herein.

FIG. 11 shows a chart depicting results showing effectiveness of the techniques described herein for estimating tumor cell on an artificial transcriptomes dataset, according to some embodiments of the technology described herein.

FIG. 12 shows graphs depicting results showing effectiveness of the techniques described herein for estimating tumor cell expression of single genes for an artificial transcriptomes dataset, according to some embodiments of the technology described herein.

FIG. 13 shows graphs depicting results showing effectiveness of the techniques described herein for estimating tumor cell gene expression on melanoma single-cell data, according to some embodiments of the technology described herein.

FIG. 14 shows graphs depicting results showing effectiveness of the techniques described herein for estimating tumor cell gene expression on lung cancer single-cell data, according to some embodiments of the technology described herein.

FIG. 15 shows graphs depicting results showing effectiveness of the techniques described herein for estimating tumor cell gene expression on head and neck cancer single-cell data, according to some embodiments of the technology described herein.

FIG. 16 shows graphs depicting results showing effectiveness of the techniques described herein for estimating tumor cell gene expression on glioblastoma single-cell data, according to some embodiments of the technology described herein.

FIG. 17 shows graphs depicting results showing effectiveness of the techniques described herein for estimating tumor cell gene expression on non-small-cell lung carcinoma single-cell data, according to some embodiments of the technology described herein.

FIG. 18 shows graphs depicting results showing effectiveness of the techniques described herein for estimating tumor cell gene expression of single genes for scRNA-seq based datasets, according to some embodiments of the technology described herein.

FIG. 19 shows graphs depicting results showing effectiveness of the techniques described herein for estimating tumor cell gene expression on datasets of in vitro mixed RNA fractions, according to some embodiments of the technology described herein.

FIG. 20 shows graphs depicting results showing effectiveness of the techniques described herein for estimating tumor cell gene expression of single genes for datasets of in vitro mixed RNA fractions, according to some embodiments of the technology described herein.

FIG. 21 shows graphs depicting results showing effectiveness of the techniques described herein for estimating tumor cell expression of the PIK3CD gene on scRNA-seq based datasets, according to some embodiments of the technology described herein.

FIG. 22 shows graphs depicting results showing effectiveness of the techniques described herein for estimating tumor cell expression of the MMP2 gene on scRNA-seq based datasets, according to some embodiments of the technology described herein.

FIG. 23 is a flowchart depicting an illustrative process for processing sequence data to obtain expression data, according to some embodiments of the technology described herein.

FIG. 24 depicts an illustrative implementation of a computer system that may be used in connection with some embodiments of the technology described herein.

### DETAILED DESCRIPTION

The inventors have developed machine learning techniques for estimating expression levels of genes in tumor cells (which may be referred to herein as “tumor expression levels”) in a biological sample (e.g., such as a sample from a tumor or other diseased tissue) based on expression data (e.g., data obtained, in part, by sequencing the biological sample, for example, using bulk RNA-sequencing). In some embodiments, the techniques involve using multiple machine learning models to estimate respective expression levels of the genes in the tumor microenvironment (TME) cells (which may be referred to herein as “TME expression levels”) of the biological sample. For example, in some embodiments, a different machine learning model may be used to estimate a respective TME expression level for each gene. In some embodiments, the outputs of the machine learning models may be used to determine respective tumor expression levels for genes in the tumor cells of the biological sample.

The inventors have appreciated that expression of particular genes by tumor cells may be used to inform tumor diagnosis, monitor disease progression, inform treatment decisions, and identify clinically-relevant biomarkers. For example, expression levels of a gene in tumor cells may be used to determine whether the tumor is of a particular type of cancer. For example, over-expression of the insulin-like growth factor 2 (IGF2) gene by tumor cells is a feature of hepatoblastoma. If the expression levels of the IGF2 gene in tumor cells are relatively high (e.g., the IGF2 gene is over-expressed), this may indicate that the tumor is of the hepatoblastoma type. Such information can be used to identify drugs known to effectively treat hepatoblastoma, to inform whether to initiate or adjust therapy, and to inform other clinical decisions related to the care of the patient. Of course, this example use of the expression levels of IGF2 should be employed only when the expression levels of IGF2 may be estimated with sufficient accuracy.

Expression levels of a gene in tumor cells may also be used to identify an effective treatment or therapy for the tumor. For example, expression of the CDK2 (cyclin dependent kinase 2) gene by tumor cells has been shown to permit immortalization of tumor cells. Due to this functionality, the CDK2 gene has been identified as a target for mechanism-based therapeutic strategies in cancer treatment. Therefore, if a patient's tumor cells are shown to express the CDK2 gene, this may indicate that the mechanism-based therapeutic strategies will effectively treat the tumor, and such therapeutic strategies may be administered to the patient.

The inventors have further recognized and appreciated that bulk sequencing, which can provide information about tens of thousands of genes in a biological sample simultaneously, can allow for the detection of a signal that represents the combined contribution of multiple cell types, including tumor cells and tumor microenvironment cells. However, the inventors have recognized that total expression data of this kind does not yield information regarding the origin of individual RNA or DNA molecules, such that there remains a significant challenge with estimating the expression level of a gene in tumor cells when that same gene is also simultaneously expressed by one or more types of TME cells. For example, PTK7 (protein tyrosine kinase 7), CCDN2 (Cyclin D2), CDK2, and IGF2 are just a few of the many genes that can be simultaneously expressed by both tumor and TME cells. Since the tumor expression of a gene can inform important decisions relating to diagnosis, prognosis, and treatment of the tumor, the inventors have recognized and appreciated that it is critical to distinguish between tumor and TME expression of genes.

Additionally, the inventors have recognized and appreciated that tumor cells may make up only a relatively small percentage of complex tumor tissue as a whole, with percentages sometimes below 10%. Measuring expression of small cell populations from bulk RNA-seq data can be especially challenging because of the reduced signal-to-noise ratio—if were to consider expression levels of tumor cells as the “signal” and expression levels of TME cells as “noise.” Moreover, because TME cellular transcripts may comprise the majority of the total transcripts in the tumor, this may lead to biases during clinical decision-making and biomarker development.

Various techniques have been employed in an attempt to estimate tumor expression of genes in a biological sample. However, such techniques have limitations and do not adequately address the above-identified issues associated with tumor expression estimation. In particular, conventional techniques involve: (a) predicting the TME expression of a gene in a biological sample based on average TME expression levels of the gene across multiple samples; and (b) subtracting the TME expression of the gene from the total expression of the gene to estimate the tumor expression of the gene. Conventional techniques for predicting the TME expression of the gene involve obtaining the average expression levels of the gene in different TME cell populations and scaling the average expression levels by a respective fraction of each of the TME cell populations. However, using average expression levels of a gene introduce inaccuracies into the predicted TME and tumor expression levels of the gene because the average levels, by definition, are not particular to an individual tumor sample—they are obtained as averages of data collected from sequencing multiple diverse samples. On the other hand, cells (e.g., tumor and TME cells) react to different environments, meaning their gene expression levels differ based on their surrounding environment. Accordingly, the average expression levels of a gene do not accurately reflect the tumor and TME expression levels of that gene in a particular tumor sample for a particular patient.

Due to the limitations in their accuracy, the output of conventional techniques cannot be used to reliably inform clinical decision making or to identify clinically-relevant biomarkers. For example, because of their reliance on average expression levels of individual genes, conventional techniques will underestimate the expression level of a gene that is uniquely, highly-expressed in TME cells of a particular tumor. Rather, the conventional techniques will inaccurately attribute this expression to tumor cells in the tumor. This could lead to, among other problems, inaccurate diagnosis, selection and administration of an ineffective treatment, and inaccurate identification of the gene as a clinically-relevant biomarker.

To address the drawbacks of conventional techniques of tumor expression estimation, the inventors have developed machine learning techniques that account for the unique expression of a particular tumor. In particular, the inventors have developed systems and methods for using machine learning to estimate tumor expression levels of genes in tumor cells in a biological sample of a subject having cancer. The developed techniques include: (a) obtaining expression data (e.g., RNA and/or DNA expression data) for genes associated with tumor cells (e.g., genes listed in Table 1) and for genes associated with TME cells (e.g., genes listed in Table 2); and (b) determining tumor expression levels for the genes associated with tumor cells using multiple machine learning models, each of which corresponds to a gene associated with tumor cells. In some embodiments, determining a tumor expression level for a particular gene associated with tumor cells involves generating a set of features for the particular gene, providing the set of features as input to a respective machine learning model (e.g., a machine learning model trained to estimate a TME expression level of the particular gene) to obtain a TME expression level estimate of the particular gene, and determining the tumor expression level for the particular gene using the TME expression level estimate and a total expression level of the gene. In some embodiments, the determined tumor expression level of the gene may be used to identify a recommended appropriate anti-cancer therapy for the subject, which therapy may then be administered.

In some embodiments, the machine learning techniques used for determining tumor expression levels include using multiple machine learning models, each trained to determine a tumor expression level for a particular respective gene. In some embodiments, the machine learning model may have multiple parameters (e.g., at least 10) and training the machine learning model may include estimating values of those parameters, computationally from training data. The training data may, in some embodiments, include real expression data obtained from sequencing samples and/or simulated expression data obtained by synthesizing these data for purposes of training using the techniques described herein. In some embodiments, generating the simulated expression data may include generating many training sets (e.g., e.g., at least 25,000, at least 50,000, at least 100,000, at least 150,000, at least 200,000, at least 500,000, etc.) for each machine learning model associated with a respective gene.

In some embodiments, the techniques developed by the inventors and described herein may be used in conjunction (e.g., onboard) with one or more sequencing platforms to immediately process the data being generated by the sequencing platforms. As a result, the data provided by the sequencing platform include accurate estimates of expression levels of genes in tumor cell and in their microenvironment. As such, the techniques described herein constitute an improvement to bioinformatics, generally and specifically, to supporting clinical decision making and understanding tumor pathogenesis because the techniques described herein provide for improved methods determining tumor expression levels of genes in tumor cells of a biological sample.

Furthermore, unlike conventional techniques, the techniques described herein account for gene expression that is particular to the biological sample by using expression data, obtained by sequencing the biological sample, as input to a machine learning model trained to estimate the tumor expression level for the particular gene. By accounting for gene expression that is particular to the biological sample, as opposed to relying solely on the average gene expression level from multiple, unrelated biological samples, the techniques determine the tumor expression level for the particular gene with greater accuracy.

Another advantage of the techniques developed by the inventors is that, in some embodiments, the models described herein have been trained with data representing artificial mixtures of cell types, allowing the training process to take into account the diverse and tissue-specific expression of tumor and TME cells across much larger numbers of samples of diverse composition (e.g., simulating a wide variety of tumor microenvironments) than could be practically possible by physically sampling and analyzing tumor samples. This substantially reduces the effort and computational resources associated with training the machine learning models for expression level estimation. The artificial mixes described herein can also be obtained in such a way that they capture a wide biological variability, improving the ability of a machine learning model trained using this data to identify biologically meaningful signals in the presence of such noise and variability. For example, as described herein, a quantitative noise model for technical noise was developed and may be applied to artificial mixes, in some embodiments. Moreover, the RNA expression data used to develop these artificial mixes was derived from multiple different samples, across multiple cell populations having a variety of biological states. These artificial mixes improve the ability of the machine learning models to effectively determine tumor expression levels for genes in tumor cells across real tumor samples.

Consequently, the techniques developed by the inventors provide for an improved diagnostic tool, which enables more accurate identification of treatments for patients, thereby improving clinical outcomes. In particular, by accurately and reliably determining the tumor expression level of a particular gene, the techniques described herein can be used to identify a treatment most effective for treating patients having that particular tumor expression level of a particular gene. By contrast, conventional techniques fail to reliably estimate tumor expression levels, resulting in unreliable and poor identification of anti-cancer treatments.

In addition to identifying therapies for a subject based on tumor expression levels using the techniques described herein, one or more clinical trials may be identified for the subject using the determined tumor expression levels.

Additionally or alternatively, the techniques described herein may be utilized in the context of quality control processes in the laboratory environment. For example, immunohistochemistry techniques may be used to initially estimate the tumor expression of a gene in tumor cells of a biological sample. However, immunohistochemistry is highly subjective since it relies on user observation of the sample under a microscope. Therefore, different users will estimate different values of tumor expression, leading to inconsistent, unreliable, and often inaccurate results. The techniques described herein may be used to objectively confirm or correct the laboratory results.

Accordingly, some embodiments provide for computer-implemented machine learning techniques for estimating tumor expression levels of genes in tumor cells in a biological sample (e.g., having tumor and TME cells) of a subject having cancer. The techniques include: (a) obtaining expression data for a set of genes, the set of genes comprising a first plurality of genes (e.g., at least one, at least some, all of the) genes shown in Table 1) associated with tumor cells and a second plurality of genes associated (e.g., at least one, at least some, all of the) genes shown in Table 2) with the tumor microenvironment cells, the expression data including first total expression levels for genes in the first plurality of genes (e.g., the combined expression of the genes by all cells in the biological sample) and second total expression levels for genes in the second plurality of genes (e.g., the combined expression of the genes by all cells in the biological sample); (b) determining the tumor expression levels (e.g., the expression levels of genes in tumor cells) of the first plurality of genes in the tumor cells using a plurality of machine learning models, the plurality of machine learning models comprising a respective machine learning model for each gene in the first plurality of genes including a first machine learning model for a first gene in the first plurality of genes, the tumor expression levels including a first tumor expression level for the first gene in the tumor cells; and (c) outputting the tumor expression levels (e.g., storing in memory, displaying a graphical user interface (GUI), transmitting to one or more devices, etc.) of the first plurality of genes in the tumor cells.

In some embodiments, determining the tumor expression levels of the first plurality of genes includes: (a) generating a first set of features for the first gene; (b) providing the first set of features as input to the first machine learning model to obtain an output indicative of a TME expression level estimate (e.g., expression level of a gene in TME cells) of the first gene in the TME cells; and (c) determining the first tumor expression level for the first gene in the tumor cells using the output of the first machine learning model and a total expression level, in the first total expression levels, for the first gene (e.g., at least in part by subtracting the TME expression level estimate from the total expression level).

In some embodiments, generating the first set of features for the first gene includes: (a) obtaining, using the expression data, an initial expression level estimate of the first gene in the tumor cells of the biological sample and including the initial expression level estimate of the first gene in the first set of features; (b) including at least some of the first total expression levels (e.g., at least 25, at least 50, at least 75, at least 100, at least 150, etc.) in the first set of features; and (c) including at least some of the second total expression levels (e.g., at least 25, at least 50, at least 75, at least 100, at least 150, etc.) in the first set of features.

In some embodiments, the plurality of machine learning models includes a second machine learning model for a second gene (e.g., one of the genes listed in Table 1) in the first plurality of genes and the tumor expression levels include a second tumor expression level for the second gene in the tumor cells. For example, the second machine learning model may be different from the first machine learning model and the second gene may be different from the first gene. In some embodiments, determining the tumor expression levels of the first plurality of genes further includes: (a) generating a second set of features for the second gene; (b) providing the second set of features as input to the second machine learning model to obtain an output indicative of a TME expression level estimate of the second gene in the TME cells; and (c) determining the second tumor expression level for the second gene in the tumor cells using the output of the second machine learning model and a total expression level, in the first total expression levels, for the second gene.

In some embodiments, generating the second set of features for the second gene includes: (a) obtaining, using the expression data, an initial expression level estimate of the second gene in the tumor cells of the biological sample and including the initial expression level estimate of the second gene in the second set of features; (b) including at least some of the first total expression levels (e.g., at least 25, at least 50, at least 75, at least 100, at least 150, etc.) in the second set of features; and (c) including at least some of the second total expression levels (e.g., at least 25, at least 50, at least 75, at least 100, at least 150, etc.) in the second set of features.

In some embodiments, the plurality of machine learning models includes a third machine learning model for a third gene (e.g., selected from the genes listed in Table 1) in the first plurality of genes and the tumor expression levels include a third tumor expression level for the third gene in the tumor cells. For example, the third machine learning model may be different from both the first and second machine learning models and the second gene may be different from both the first and second genes. In some embodiments, determining the tumor expression levels of the first plurality of genes further includes (a) generating a third set of features for the third gene, (b) providing the third set of features as input to the third machine learning model to obtain an output indicative of a TME expression level estimate of the third gene in the TME cells, and (c) determining the third tumor expression level for the third gene in the tumor cells using the output of the third machine learning model and a total expression level, in the first total expression levels, for the third gene.

In some embodiments, generating the first set of features for the first gene further comprises obtaining, using the expression data, a first plurality of RNA percentages (e.g., by cellular deconvolution) for a respective plurality of types of cells that occur in the TME, wherein each of the first plurality of RNA percentages indicates a percent of RNA (e.g., in the biological sample) associated with the first gene (e.g., produced during expression of the first gene) and originating (e.g., produced by) cells of a respective type (e.g., neutrophils, fibroblasts, etc.) in the biological sample. For example, in some embodiments, obtaining the first plurality of RNA percentages includes processing at least some of the expression (e.g., a portion or all of the expression data) using at least one non-linear regression model.

In some embodiments, generating the first set of features for the first gene further comprises including at least some of the first plurality of RNA percentages in the first set of features

In some embodiments, the TME cells comprise TME cells of a first type and TME cells of a second type (e.g., different from the first type). In some embodiments, the at least some of the expression data includes a first subset of the expression data and a second subset (e.g., different from the first subset) of the expression data. In some embodiments, the at least one non-linear regression model includes a first non-linear regression model and a second non-linear regression model different from the first non-linear regression model. In some embodiments, obtaining the first plurality of RNA percentages includes (a) processing the first subset of the expression data using the first non-linear regression model to obtain a first RNA percentage for the TME cells of the first type; and (b) processing the second subset of the expression data using the second non-linear regression model to obtain a second RNA percentage for the TME cells of the second type.

In some embodiments, the first type of TME cells and second type of TME cells are each selected from the group consisting of B cells, CD4+ T cells, CD8+ T cells, endothelial cells, fibroblasts, lymphocytes, macrophages, monocytes, NK cells, and neutrophils, wherein the first type is different from the second type. However, it should be appreciated that the cell type could be any suitable type of TME cell, as aspects of the technology described herein are not limited to any particular type of TME cell.

In some embodiments, obtaining the initial expression level estimate of the first gene in the tumor cells of the biological sample includes (a) obtaining an average TME expression level (e.g., obtained based on previously-determined expression levels of the first gene in TME cells of different biological samples) of the first gene for each of the plurality of types of cells that occur in the TME; (b) determining a weighted sum of the obtained expression levels based on the first plurality of RNA percentages (e.g., by multiplying the first plurality of RNA percentages with respective average expression levels); and (c) subtracting the weighted sum from the total expression level for the first gene to obtain the initial expression level estimate.

In some embodiments, the techniques further include obtaining, using the expression data, a first RNA percentage for the tumor cells, wherein the first RNA percentage indicates a percent of RNA associated with the first gene and originating from the tumor cell of the biological sample. For example, the first RNA percentage may be obtained using the techniques for obtaining RNA percentages for the types of cells that occur in the TME.

In some embodiments, the expression data has been previously obtained at least in part by sequencing (e.g., RNA or DNA sequencing) the biological sample of the subject having cancer.

In some embodiments, the at least some of the first total expression levels included in the first set of features include total expression levels for at least 25 genes, at least 50 genes, at least 75 genes, at least 100 genes, or at least 150 genes in the first plurality of genes associated with tumor cells. In some embodiments, the plurality of machine learning models comprises at least 25 machine learning models, at least 50 machine learning models, at least 75 machine learning models, at least 100 machine learning models, or at least 150 machine learning models corresponding to the at least 25 genes, at least 50 genes, at least 75 genes, at least 100 genes, or at least 150 genes, respectively.

In some embodiments, each machine learning model of the at least 25 machine learning models (at least 50 machine learning models, at least 75 machine learning models, at least 100 machine learning models, or at least 150 machine learning models, etc.) comprises a different gradient boost model.

In some embodiments, the at least some of the first total expression levels included in the first set of features include total expression levels for at least 10 genes selected from genes listed in Table 1. In some embodiments, the at least some of the first total expression levels included in the first set of features include total expression levels for at least 25 genes selected from genes listed in Table 1. In some embodiments, the at least some of the first total expression levels included in the first set of features include total expression levels for at least 50 genes selected from genes listed in Table 1. In some embodiments, the at least some of the first total expression levels included in the first set of features include total expression levels for at least 75 genes selected from genes listed in Table 1. In some embodiments, the at least some of the first total expression levels included in the first set of features include total expression levels for at least 100 genes selected from genes listed in Table 1. In some embodiments, the at least some of the first total expression levels included in the first set of features include total expression levels for at least 150 genes selected from genes listed in Table 1.

In some embodiments, the first machine learning model of the plurality of machine learning models is a gradient boosted model (e.g., trained using a gradient boosting framework such as LightGBM, Catboost, XGBoost, Adaboost, etc.).

In some embodiments, the techniques further include training the first machine learning model by (a) obtaining training data comprising simulated expression data for genes in the set of genes, wherein the training data is associated with one or more biological samples (e.g., tumor and/or non-tumor samples obtained from one or more subjects); (b) generating, using the training data, a training set of features for the first gene; and (c) training the first machine learning model to estimate a TME expression level of the first gene. In some embodiments, the training includes providing the training set of features as input to the first machine learning model to obtain an output comprising an estimate of the TME expression level of the first gene in the TME cells of the one or more biological samples and updating parameters of the first machine learning model using the estimate of the TME expression level.

In some embodiments, generating the training set of features for the first gene includes obtaining, using the simulated expression data, an initial expression level estimate of the first gene in tumor cells of the one or more biological samples and including the initial expression level estimate in the training set of features and including at least some of the simulated expression levels in the training set of features (e.g., at least some expression levels of genes associated with tumor cells and at least some expression levels of genes associated with TME cells).

In some embodiments, the first machine learning model was trained at least in part by generating training data comprising simulated expression data. In some embodiments, generating the training data includes (a) obtaining training expression data for each of one or more biological samples, the training expression data comprising first training expression levels for the first plurality of genes (e.g., associated with tumor cells) and second training expression levels for the second plurality of genes (e.g., associated with TME cells); (b) generating first simulated expression data using the first training expression levels; (c) generating second simulated expression data using the second training expression levels; and (d) combining the first simulated expression data and the second simulated expression data to produce at least part of the simulated expression data.

In some embodiments, the techniques further include identifying at least one anti-cancer therapy for the subject based on the first tumor expression level for the first gene in the tumor cells. For example, an anti-cancer therapy may be identified for the subject if the first tumor expression level satisfies some criteria (e.g., falls within a range of expression levels, exceeds a threshold expression level, is lower than a threshold expression level, etc.). In some embodiments, the techniques further comprise administering the at least one anti-cancer therapy.

In some embodiments, the at least one anti-cancer therapy is selected from the group of therapies for the first gene listed in Table 3.

In some embodiments, identifying the at least one anti-cancer therapy includes determining whether the first tumor expression level satisfies at least one criterion associated with the first gene and after determining that the first tumor expression level satisfies the at least one criterion, selecting the at least one anti-cancer therapy from the group of therapies listed for the first gene in Table 3. For example, the at least one criterion may be particular to the first gene.

Following below are more detailed descriptions of various concepts related to, and embodiments of, the cellular deconvolution systems and methods developed by the inventors. It should be appreciated that various aspects described herein may be implemented in any of numerous ways. Examples of specific implementations are provided herein for illustrative purposes only. In addition, the various aspects described in the embodiments below may be used alone or in any combination and are not limited to the combinations explicitly described herein.

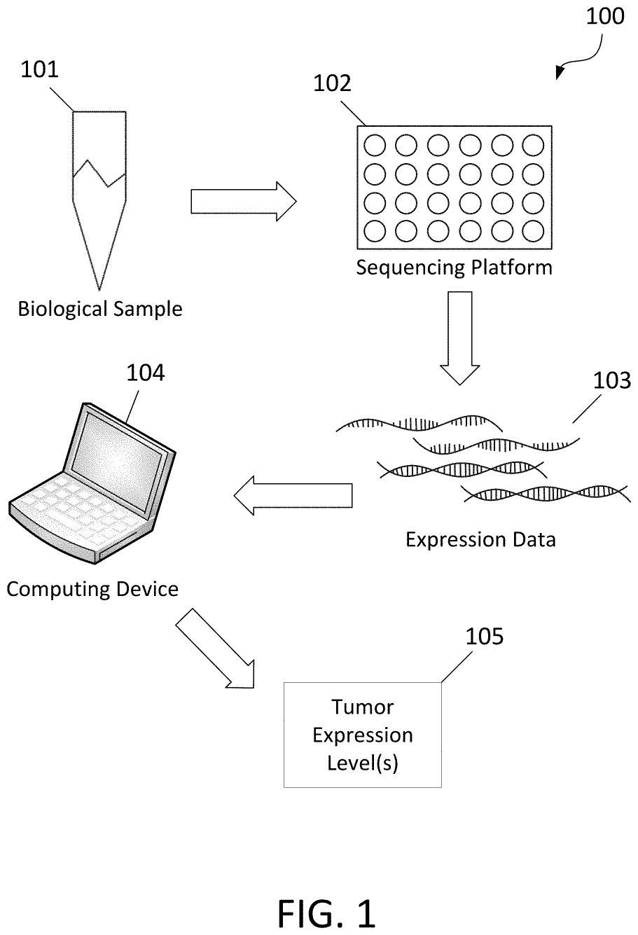

FIG. 1 depicts an illustrative technique 100 for estimating tumor expression level(s) 105 of genes in tumor cells in a biological sample 101 based on expression data 103 obtained using sequencing platform 102 to process biological sample 101. The tumor expression level(s) are determined by processing the expression data 103 using computing device 104.

In some embodiments, the illustrative technique 100 may be implemented in a clinical or laboratory setting. For example, the technique 100 may be implemented on a computing device 104 that is located within the clinical or laboratory setting. In some embodiments, the computing device 104 may directly obtain the expression data 103 from a sequencing platform 102 located within the clinical or laboratory setting. For example, a computing device 104 included in the sequencing platform 102 may directly obtain the expression data 103 via a communication network, such as Internet or any other suitable network, as aspects of the technology described herein are not limited to any particular communication network.

Additionally or alternatively, the illustrative technique 100 may be implemented in a setting that is remote from a clinical or laboratory setting. For example, the illustrated technique 100 may be implemented on computing device 104 that is located externally from a clinical or laboratory setting. In this case, the computing device may indirectly obtain expression data 103 that is generated using a sequencing platform 102 located within or external to a clinical or laboratory setting. For example, the expression data 103 may be provided to computing device 104 via a communication network, such as Internet or any other suitable network, as aspects of the technology described herein are not limited to any particular communication network.

As shown in FIG. 1, the technique 100 involves processing the biological sample 101 using a sequencing platform 102, which produces expression data 103. The biological sample 101 may be obtained from a subject having, suspected of having, or at risk of having cancer. The biological sample 101 may be obtained by performing a biopsy or by obtaining a blood sample, a salivary sample, or any other suitable biological sample from the subject. The biological sample 101 may include diseased tissue (e.g., cancerous) and/or healthy tissue (e.g., non-tumorous). The biological sample may include tumor cells and/or TME cells. Different types of cells occur in the TME. For example, the TME may include, as nonlimiting examples, B cells, CD4+ T cells, CD8+ T cells, endothelial cells, fibroblasts, lymphocytes, macrophages, monocytes, NK cells, and neutrophils. In some embodiments, the origin or preparation methods of the biological sample may include any of the methods described herein including in the “Biological Samples” section.

In some embodiments, the sequencing platform 102 may be a next generation sequencing platform (e.g., Illumina™, Roche™, Ion Torrent™, etc.), or any high-throughput or massively parallel sequencing platform. In some embodiments, the sequencing platform 102 may include any suitable sequencing device and/or any sequencing system including one or more devices. In some embodiments, the sequencing methods may be automated, in some embodiments, there may be manual intervention. In some embodiments, the expression data 103 may be obtained using techniques other than next generation sequencing (e.g., Sanger sequencing, microarrays, etc.).

Expression data 103 may include the sequence data generated by a sequencing protocol (e.g., the series of nucleotides in a nucleic acid molecule identified by next-generation sequencing, Sanger sequencing, etc.) as well as information contained therein (e.g., information indicative of source, tissue type, etc.) which may also be considered information that can be inferred or determined from the sequence data. In some embodiments, expression data 103 may include information included in a FASTA file, a description and/or quality scores included in a FASTQ file, an aligned position included in a BAM file, and/or any other suitable information.

The expression data 103 may be generated by sequencing biological sample 101. Biological sample 101 may include nucleic acid. A nucleic acid may include one or multiple nucleic acid molecules.

In some embodiments, the nucleic acid is RNA. In some embodiments, sequenced RNA comprises both coding and non-coding transcribed RNA found in a sample. When such RNA is used for sequencing the sequencing is said to be generated from “total RNA” and also can be referred to as whole transcriptome sequencing. Alternatively, the nucleic acids can be prepared such that the coding RNA (e.g., mRNA) is isolated and used for sequencing. This can be done through any means known in the art, for example by isolating or screening the RNA for polyadenylated sequences. This is sometimes referred to as mRNA-Seq.

In some embodiments, the nucleic acid is DNA. In some embodiments, the nucleic acid is prepared such that the whole genome is present in the nucleic acid. In some embodiments, the nucleic acid is processed such that only the protein coding regions of the genome remain (e.g., the exome). When nucleic acids are prepared such that only the exome is sequenced, it is referred to as whole exome sequencing (WES). A variety of methods are known in the art to isolate the exome for sequencing, for example, solution-based isolation wherein tagged probes are used to hybridize the targeted regions (e.g., exons) which can then be further separated from the other regions (e.g., unbound oligonucleotides). These tagged fragments can then be prepared and sequenced.

In some embodiments, expression data 103 may include raw DNA or RNA sequence data, DNA exome sequence data (e.g., from whole exome sequencing (WES), DNA genome sequence data (e.g., from whole genome sequencing (WGS)), RNA expression data, gene expression data, bias-corrected gene expression data, or any other suitable type of sequence data comprising data obtained from the sequencing platform 102 and/or comprising data derived from data obtained from sequencing platform 102. In some embodiments, the origin or preparation of the expression data 103 may include any of the embodiments described with respect to the “Expression Data” and “Obtaining Expression Data” sections.

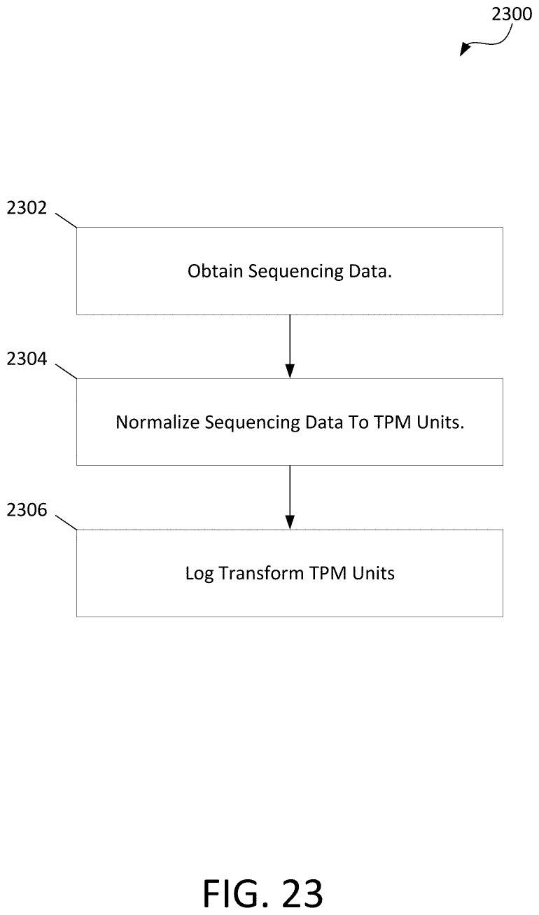

In some embodiments, the expression data 103 includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject. Example techniques for processing sequencing data to obtain expression data, including expression levels, are described herein including at least with respect to FIG. 23 and the section “Expression Levels.”

In some embodiments, the gene expression levels include total expression levels. As referred to herein, the “total expression level” for a gene is a numeric value quantifying the degree to which the gene is expressed in the biological sample 101. The total expression level for a gene may reflect the combined expression of the gene in both tumor and TME cells of the biological sample. As such, the total expression level for a particular gene may not distinguish between the expression of that particular gene in tumor cells and the expression of that particular gene in TME cells.

In some embodiments, a total expression level is obtained for each of multiple genes. For example, total expression levels may be obtained for at least 10 genes, at least 25 genes, at least 50 genes, at least 75, genes, at least 100 genes, at least 150 genes, at least 200 genes, at least 250 genes, at least 300 genes, at least 350 genes, at least 400 genes, at least 450 genes, at least 500 genes, at least 550 genes, at least 600 genes, or more genes.

In some embodiments, the genes include genes associated with tumor cells and genes associated with TME cells. In some embodiments, genes “associated with tumor cells” include those that are predominantly expressed in tumor cells. Nonlimiting examples of genes associated with the tumor cells include those listed in Table 1. In some embodiments, genes “associated with TME cells” include those that are predominantly expressed in TME cells. Nonlimiting examples of genes associated with TME cells include those listed in Table 2.

In some embodiments, the expression data 103 includes total expression levels for at least some of the genes associated with tumor cells and at least some of the genes associated with TME cells. For example, expression data 103 may include total expression levels for at least 10, at least 25, at least 30, at least 40, at least 50, at least 60, at least 75, at least 100, at least 150, or more genes associated with tumor cells. The genes may be selected, for example, from those listed in Table 1. Additionally or alternatively, expression data 103 may include total expression levels for at least 10, at least 25, at least 30, at least 40, at least 50, at least 60, at least 75, at least 100, at least 150, at least 200, at least 250, at least 300, at least 350, at least 400, or more genes associated with TME cells. The genes may be selected, for example, from those listed in Table 2.

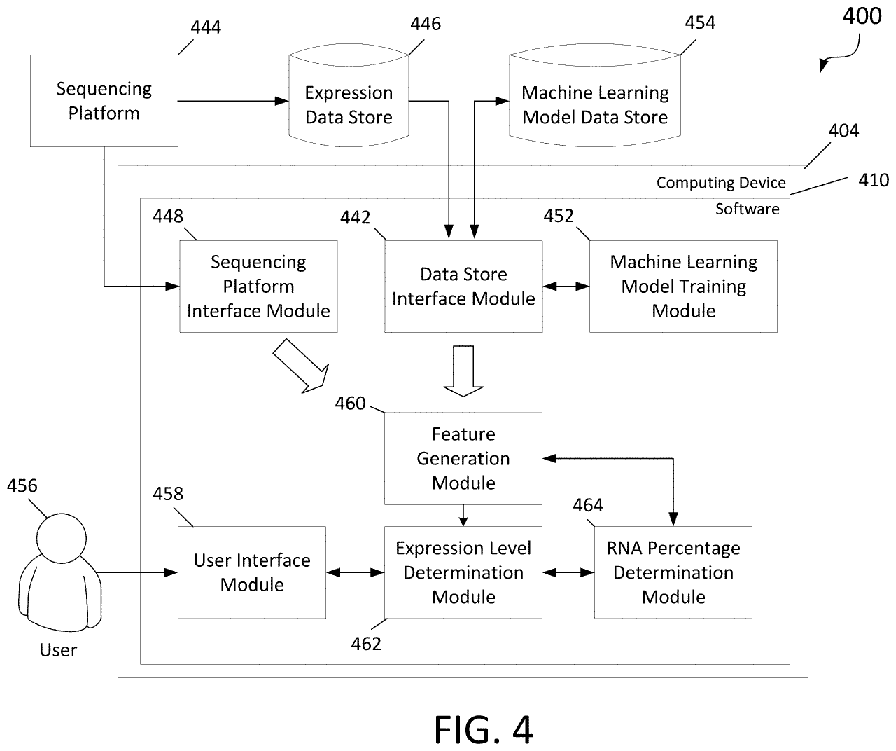

Regardless of the type of expression data 103 obtained, the expression data 103 is processed using computing device 104. The computing device 104 can be one or multiple computing devices of any suitable type. For example, the computing device 104 may be a portable computing device (e.g., laptop, a smartphone) or a fixed computing device (e.g., a desktop computer, a server). When computing device 104 includes multiple computing devices, the device(s) may be physically co-located (e.g., in a single room) or distributed across multiple physical locations. In some embodiments, the computing device 104 may be part of a cloud computing infrastructure. In some embodiments, one or more computer(s) 104 may be co-located in a facility operated by an entity (e.g., a hospital, a research institution). In some embodiments, the one or more computing device(s) 104 may be physically co-located with a medical device, such as a sequencing platform 102. For example, a sequencing platform 102 may include computing device 104. FIG. 4 shows a system 400 including example computing device 404 and software 410.

In some embodiments, the computing device 104 may be operated by a user such as a doctor, clinician, researcher, patient, or other individual. For example, the user may provide the expression data 103 as input to the computing device 104 (e.g., by uploading a file), and/or may provide user input specifying processing or other methods to be performed using the expression data 103.

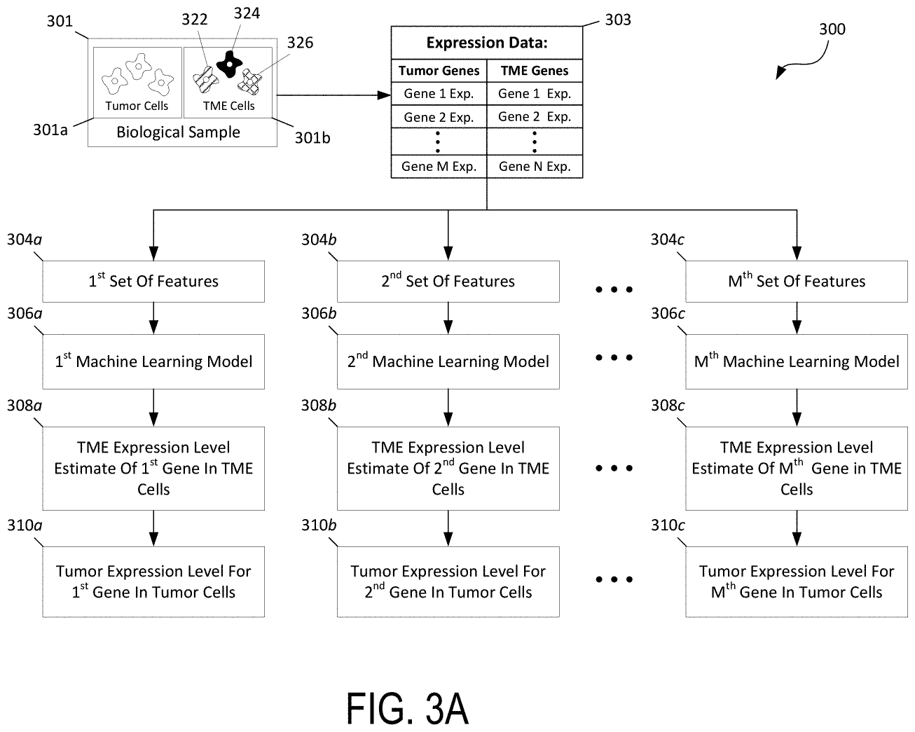

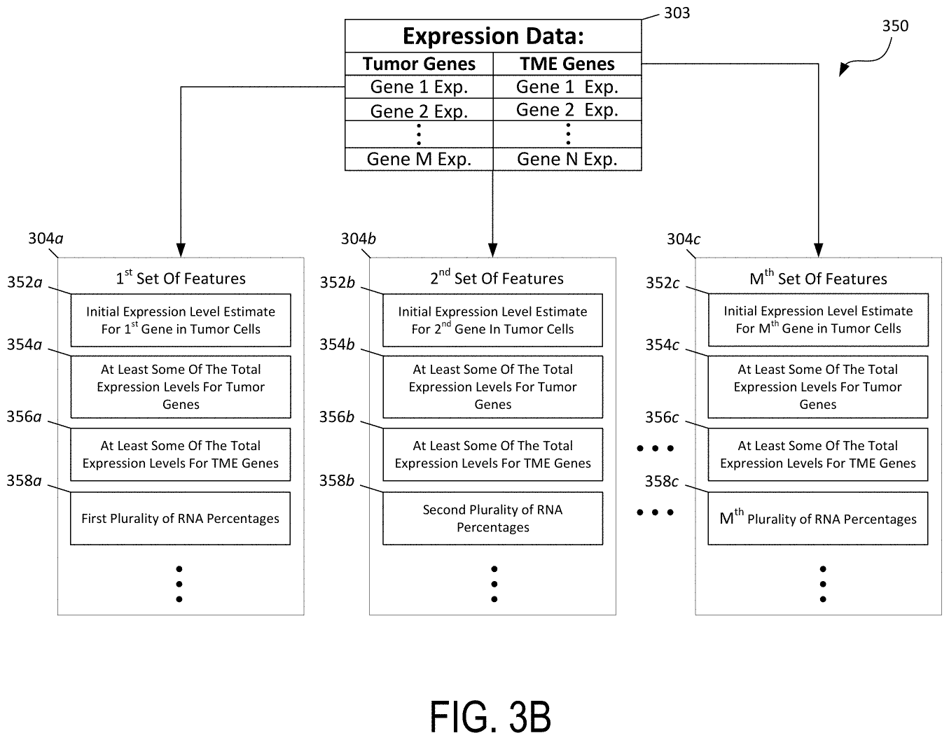

In some embodiments, expression data 103 may be processed by one or more software programs running on computing device 104 (e.g., as described herein including at least with respect to FIG. 4). In particular, in some embodiments, expression data 103 is used to generate sets of features that are provided as inputs to a plurality of machine learning models corresponding to a respective plurality of genes associated with tumor cells (e.g., genes listed in Table 1). For example, the expression data 103 may be used to generate a first set of features (e.g., first set of features 304a shown in FIGS. 3A-3B) for a first gene associated with tumor cells, and the first set of features may be provided as input to a first machine learning model (e.g., first machine learning model 306a shown in FIGS. 3A-3B) corresponding to the first gene. Additionally, the expression data 103 may be used to generate a second set of features (e.g., second set of features 304b shown in FIGS. 3A-3B) for a second gene associated with tumor cells, and the second set of features may be provided as input to a second machine learning model (e.g., second machine learning model 306b shown in FIGS. 3A-3B) corresponding to the second gene. Such processing may be performed for each of multiple genes associated with tumor cells. For example, expression data 103 may be used to generate M sets of features that are provided as inputs to M machine learning models, where M is at least 10, at least 20, at least 25, at least 30, at least 35, at least 40, at least 45, at least 50, at least 50, at least 75, at least 100, at least 120, between 10 and 130, between 20 and 100, between 25 and 75, etc.

In some embodiments, each of the plurality of machine learning models is of any suitable type. For example, each of the machine learning models may be a gradient boosted machine learning model (e.g., a first gradient boosted machine learning model, a second gradient boosted machine learning model, etc). The gradient boosted machine learning model may be a gradient boosted decision tree model or using any other suitable type of model as “weak learner” boosted via gradient boosting or any other suitable boosting approach. In some embodiments, the gradient boosted ML model may be trained using a gradient boosting framework such as XGBoost, LightGBM, Catboost, or Adaboost.

It should be appreciated that a machine learning model of the plurality of machine learning models need not be a gradient boosted machine learning model and that other types of machine learning models may be used. For example, in some embodiments, a non-linear regression model (e.g., a logistic regression model), a neural network model, a support vector machine, a Gaussian mixture model, a random forest model, a decision tree model, or any other suitable type of machine learning model, as aspects of the technology described herein are not limited in this respect.

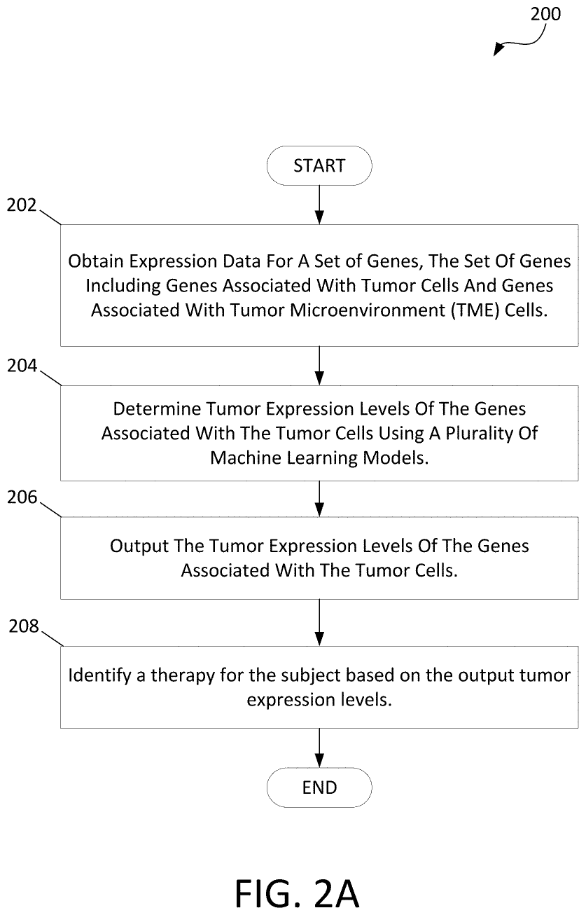

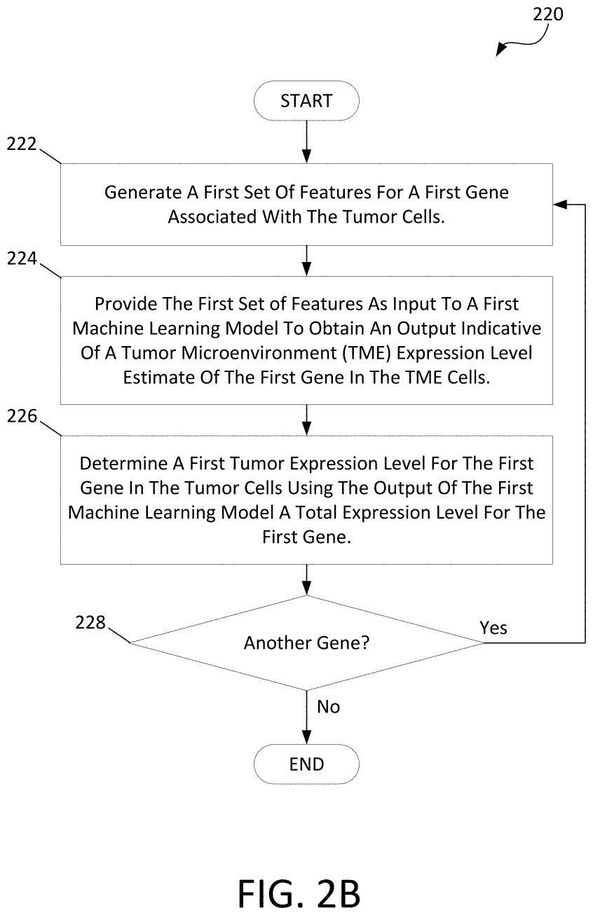

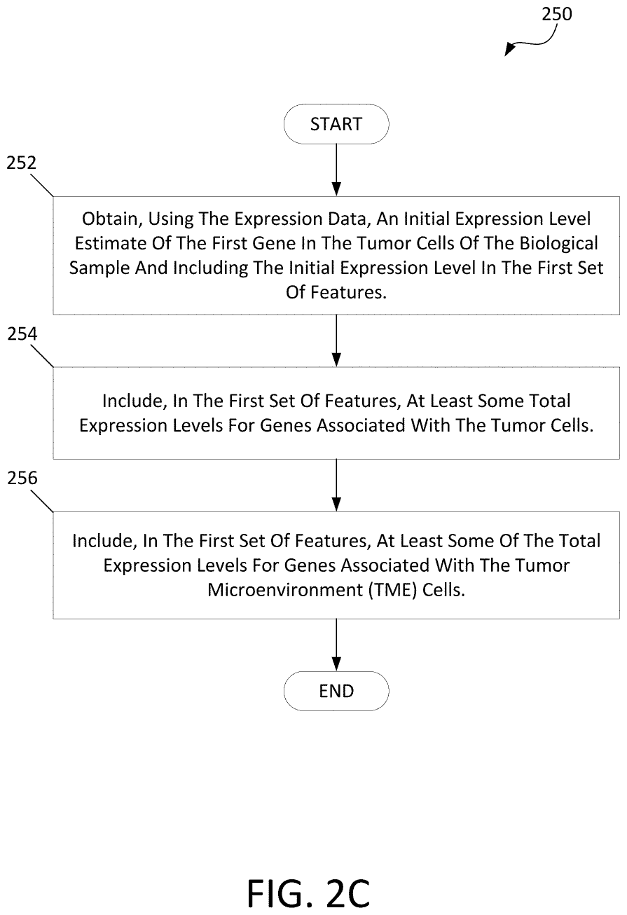

In some embodiments, a machine learning model is trained to estimate a TME expression level of a gene associated with tumor cells. As referred to herein, the “TME expression level” of a gene is a numeric value quantifying the degree to which the gene is expressed in TME cells of a biological sample. For example, a first machine learning model may be trained to estimate a TME expression level of a first gene in the biological sample 101 and a second machine learning model may be trained to estimate a TME expression level of a second gene in the biological sample 101. Illustrative techniques for processing the expression data to estimate TME expression levels are described herein, including at least with respect to act 224 of process 220, shown in FIG. 2B.

Based on the outputs of the machine learning models, including the output of the first machine learning model, in some embodiments, tumor expression level(s) 105 are determined for at least one of the genes associated with tumor cells. For example, the tumor expression level(s) 105 may include a first tumor expression level for a first gene associated with tumor cells. As referred to herein, the “tumor expression level” of a gene is a numeric value quantifying the degree to which the gene is expressed in tumor cells of a biological sample. Illustrative techniques for processing the expression data to estimate tumor expression levels are described herein, including at least with respect to act 226 of process 220, shown in FIG. 2B.

In some embodiments, the tumor expression level(s) 105 may be provided as output. For example, the tumor expression level(s) 105 may be used to generate a report to be output to a user (e.g., via a graphical user interface (GUI).

In some embodiments, the tumor expression level(s) 105 may be used to identify a tumor-specific treatment for the subject from which the biological sample 101 was obtained. For example, the expression of a gene may be associated with at least one treatment known to be effective in treating tumors that express that gene (e.g., at a particular expression level). Such a treatment may be identified to treat the biological sample 101 and, in some embodiments, subsequently administered to the subject. For example, Table 3 lists treatments associated respectively with the expression of particular genes associated with tumor cells.

Additionally or alternatively, the tumor expression level(s) 105 may be used to confirm tumor expression levels previously estimated for the biological sample 101. For example, immunohistochemistry results may be received from a lab or a clinical setting. The illustrative techniques 100 may include comparing the immunohistochemistry results to the tumor expression level(s) 105 determined for the biological sample 101. If the expression levels do not match, this may indicate that the biological sample 101 used to obtain the tumor expression level(s) 105 is not reliable or that the immunohistochemistry results are not reliable. Therefore, discrepancies between the obtained expression levels can be used to identify issues of quality control, which may be reported back to the appropriate lab or clinical setting.

TABLE 1
Genes Associated with Tumor Cells

| | |
|---|---|
| NF1 | NM_001042492; NM_000267; NM_001128147 |
| CCNE1 | XM_011527440; NM_001238; NM_001322259; NM_001322261; XM_047439606; |
| | NM_001322262; NM_057182 |
| PLK1 | NM_005030 |
| ERBB4 | XM_005246376; XM_017003577; XM_017003578; XM_005246377; NM_001042599; |
| | XM_017003581; XM_006712364; XM_017003582; XM_017003579; XM_017003580; |
| | NM_005235 |
| NF2 | XM_047441386; NM_181828; NM_181830; NM_181826; NM_000268; NR_156186; |
| | NM_181827; NM_181834; NM_016418; NM_181829; NM_181825; NM_181831; |
| | NM_181835; XM_017028809; NM_181832; NM_181833 |
| XRCC1 | NM_006297 |
| MAGEA1 | NM_004988 |
| PDGFA | XM_011515415; XM_011515419; XM_011515418; NM_001395365; NR_172526; |
| | XM_011515416; XM_047420455; XM_047420458; NM_001395363; NM_001395364; |
| | NM_033023; XM_017012289; NM_001395366; XM_047420457; NR_172527; |
| | XM_047420456; NM_002607 |
| HDAC2 | NR_033441; XM_047418692; NR_073443; NM_001527 |
| BCL2L2 | NM_004050; NM_001199839 |
| NOTCH3 | XM_005259924; NM_000435 |
| TUBB3 | NM_006086; NM_001197181 |
| AURKB | NM_001313950; NM_001313953; XM_017025311; XM_047437050; NM_001313952; |
| | NM_004217; NM_001313954; NR_132730; NR_132731; NM_001284526; XM_047437051; |
| | XM_011524072; NM_001256834; NM_001313951; NM_001313955 |
| CCND2 | NM_001759 |
| CDKN2A | XM_011517676; XM_011517675; NM_001363763; NM_001195132; XM_047422597; |
| | NM_058195; XM_047422596; XM_047422598; NM_000077; NM_058196; NM_058197 |
| CCNE2 | XM_047422411; XM_017013958; NM_057749; XM_011517366; XM_017013959; |
| | NM_004702; NM_057735 |
| ROR2 | XM_005252008; XM_017014762; XM_047423434; XM_047423436; XM_006717121; |
| | XM_047423435; NM_004560; XM_005252009; XM_047423437; NM_001318204 |
| RRM2 | NM_001034; NR_164157; NR_161344; NM_001165931 |
| UMPS | NR_033437; XR_001740253; NR_033434; NM_000373 |
| CIITA | XM_047434115; NM_001379332; XR_007064880; XM_006720880; XM_011522491; |
| | XM_047434119; NM_001379334; XM_047434118; XM_047434120; XM_047434123; |
| | NM_001379333; XM_011522486; NM_000246; NM_001286402; XM_047434122; |
| | XM_047434126; XR_001751904; XR_007064879; XM_047434114; XM_047434117; |
| | XM_047434125; NM_001286403; NM_001379331; XM_011522485; XM_047434127; |
| | XM_047434128; NR_104444; XM_011522484; XM_011522490; XM_047434116; |
| | XM_047434124; NM_001379330 |
| HDAC4 | XM_011512219; XM_011512225; XM_047446479; XM_047446483; XM_047446487; |
| | NM_001378415; XM_011512218; XM_017005394; XM_047446484; XM_047446490; |
| | XM_047446492; XM_047446494; XM_011512224; XM_047446477; XM_047446478; |
| | XM_047446480; XM_047446493; XM_047446496; NM_001378416; NM_006037; |
| | XM_011512223; XM_011512227; XM_047446482; NM_001378414; XM_011512220; |
| | XM_011512222; XM_024453257; XM_047446485; XM_047446486; XM_047446489; |
| | XM_047446495; XM_011512217; XM_011512226; XM_047446476; XM_047446491; |
| | XM_047446497; XM_047446498; NM_001378417; XM_006712877; XM_006712880; |
| | XM_047446481; XM_047446488 |
| DPYD | XM_006710397; XM_017000507; XM_047448077; NM_000110; NM_001160301; |
| | XM_047448076; XR_001737014; XM_005270562 |
| AKT2 | XM_011526616; XM_047438397; NM_001626; XM_047438398; XM_047438403; |
| | XM_011526619; XM_047438399; XM_047438401; NM_001243027; XM_011526618; |
| | NM_001243028; NM_001330511; XM_011526614; XM_047438400; XM_047438402; |
| | XM_011526615 |
| PIK3CD | XM_024447663; XM_047422552; XM_047422561; XM_047422568; XM_047422573; |
| | XM_047422574; XM_047422575; XM_047422577; XM_024447664; XM_047422553; |
| | XM_047422564; XM_047422566; NM_005026; XM_047422567; XM_047422569; |
| | NM_001350234; XM_047422554; XM_047422555; XM_047422589; XM_006710689; |
| | XM_047422550; XM_047422557; XM_006710687; XM_047422558; XM_047422559; |
| | XM_047422563; XM_047422565; XM_047422580; XM_047422551; XM_047422556; |
| | XM_047422562; XM_047422570; XM_047422571; NM_001350235; XM_047422560; |
| | XM_047422572; XM_047422576; XM_047422578 |
| AURKA | XM_047440427; XM_047440428; NM_001323304; NM_001323303; NM_198435; |
| | NM_198437; NM_198433; NM_198434; NM_198436; XM_017028034; XM_017028035; |
| | NM_001323305; NM_003600 |
| ATR | XM_047448362; XM_011512925; NM_001354579; XM_047448361; XM_011512924; |
| | XM_047448363; NM_001184; XM_047448364; XM_047448360 |
| EREG | NM_001432 |
| FGFR1 | XM_024447097; XM_047421569; XM_047421570; NM_001174065; NM_001354370; |
| | NM_023111; XM_006716303; XM_006716304; XM_006716310; XM_011544445; |
| | XM_011544449; XM_017013221; XM_017013225; NM_001354368; NM_001354369; |
| | NM_015850; NM_023106; XM_006716307; XM_011544444; XM_047421571; |
| | XM_047421572; NM_001354367; NM_023105; XM_00671631 1; XM_011544446; |
| | XM_011544452; XM_017013219; XM_017013226; XM_047421573; XM_047421574; |
| | NM_023107; NM_023109; XM_011544447; XM_011544451; NM_023110; |
| | XM_006716312; XM_011544450; XM_017013220; XM_017013227; XM_017013231; |
| | NM_001174067; NM_032191; XM_006716314; XM_011544448; XM_047421575; |
| | NM_001174063; NM_001174064; NM_001174066; XM_047421576; NM_023108 |
| HDAC9 | NM_001204147; NM_001321868; NM_001321878; NM_001321887; NM_001321891; |
| | NM_001321897; NM_058177; NM_001204144; NM_001321873; NM_001321879; |
| | NM_001321884; NR_135835; NM_001321890; NM_001321894; NM_001321898; |
| | NM_001321900; NM_014707; NM_178425; NM_001321874; NM_001321877; |
| | NM_001321888; NM_001321895; NM_058176; NM_001321869; NM_001321885; |
| | NM_001321886; NM_001321899; NM_001321901; NM_001321902; NM_178423; |
| | NM_001204146; NM_001204148; NM_001321870; NM_001321893; NM_001321871; |
| | NM_001321875; NM_001204145; NM_001321872; NM_001321876; NM_001321889; |
| | NM_001321896 |
| MAGEA2 | NM_001386130.2; NM_005361.3; NM_175742.2; NM_175743.2; NM_001282501.2; |
| | NM_001282502.1; NM_001282504.1; NM_001282505.1 |
| FLNA | NM_001110556.2; NM_001456.4 |
| SLC39A6 | NM_001099406; NM_012319 |
| FLT1 | NM_001160030; NM_001159920; XM_011535014; XM_017020485; NM_001160031; |
| | NM_002019 |
| CD22 | NM_001185100; NM_001185099; NM_024916; NM_001185101; NM_001771; |
| | NM_001278417 |
| ALK | NM_004304; NM_001353765; XR_001738688 |
| PGR | XM_011542869; NM_001271161; NR_073142; XM_006718858; NM_000926; |
| | NM_001202474; NM_001271162; NR_073141; NR_073143 |
| TP53 | NM_000546; NM_001126112; NM_001276695; NM_001126115; NM_001126116; |
| | NM_001126118; NM_001276697; NM_001276698; NM_001276760; NM_001276761; |
| | NM_001126114; NM_001276696; NM_001126113; NM_001126117; NM_001276699 |
| FGFR2 | XM_017015924; NM_001144919; XM_006717708; XM_017015925; NM_001144915; |
| | NM_001144917; NM_022975; NM_023028; XM_024447890; NM_000141; |
| | NM_001144913; NM_001320654; NM_022970; NR_073009; NM_022971; NM_022973; |
| | NM_023030; XM_006717710; XM_024447887; XM_024447888; NM_001320658; |
| | NM_022976; XM_017015920; NM_001144918; NM_022974; NM_023031; |
| | XM_024447889; XM_024447891; NM_023029; XM_017015921; NM_001144914; |
| | NM_001144916; NM_022972 |
| TXNRD1 | NM_001261446; NM_182742; NM_182743; NM_003330; NM_182729; NM_001093771; |
| | NM_001261445 |
| STK11 | NM_000455 |
| MAGEA3 | XM_011531161; XM_005274676; XM_006724818; XM_011531160; NM_005362 |
| CDKN1A | NM_001220778; NM_001374510; NM_078467; NR_164655; NM_001291549; |
| | NM_001374511; NM_001374509; NR_164656; NM_000389; NM_001220777; |
| | NM_001374512; NM_001374513 |
| MAGEA4 | NM_001386196; NM_001386197; NM_001386200; NM_002362; NM_001011550; |
| | NM_001386202; NM_001011548; NM_001011549; NM_001386198; NM_001386203; |
| | NM_001386199 |
| NTRK3 | XM_006720550; XR_001751292; XM_024449935; XM_047432602; NM_001375813; |
| | XR_002957645; XM_017022245; XM_017022252; XM_024449934; NM_001375812; |
| | XM_006720549; XM_017022241; XM_017022250; NM_001320135; XM_017022240; |
| | XM_047432603; NM_001012338; XM_006720545; XM_011521638; XM_017022244; |
| | XM_017022251; XM_047432604; NM_001007156; NM_001243101; XM_017022242; |
| | NM_001320134; NM_001375810; NM_001375814; NM_002530; XM_006720548; |
| | XM_017022243; XM_017022254; NM_001375811; XR_001751293 |
| TERT | NR_149162; NM_198255; NM_198253; NR_149163; NM_001193376; NM_198254 |
| CDK4 | NM_000075; NM_052984 |
| XRCC5 | NM_021141 |
| B2M | XM_005254549; NM_004048 |
| CHEK2 | XM_006724114; XM_011529845; XM_024452148; XM_047441105; XM_047441106; |
| | NM_001349956; XM_006724116; XR_007067954; XM_017028560; XM_047441104; |
| | NM_001257387; NM_007194; XM_011529842; XM_047441108; NM_145862; |
| | XM_011529839; XM_011529844; XM_024452149; XM_047441107; XR_937806; |
| | XR_937807; XM_011529840; NM_001005735; XR_007067955 |
| TSC2 | XM_047434556; NM_021056; NM_001318831; XM_047434555; XM_011522637; |
| | NM_001077183; NM_001318832; NM_001363528; XM_011522639; XM_017023615; |
| | XM_047434557; NM_001318827; NM_001370405; XM_011522636; XM_011522640; |
| | NM_000548; NM_001370404; NM_021055; XM_011522638; NM_001114382; |
| | NM_001318829 |
| EGF | XM_017007848; XM_005262796; XM_011531707; XM_017007850; XM_047449723; |
| | NM_001178131; XM_047449725; XM_017007847; XM_017007855; XM_047449726; |
| | XM_047449727; XM_047449729; XM_017007854; NM_001963; XR_001741156; |
| | XM_017007845; XM_017007849; XM_047449728; NM_001178130; XM_017007846; |
| | XM_017007853; NM_001357021; XM_017007851; XM_047449724; XM_047449730 |
| ABCC3 | NM_001144070; NM_003786; NM_020037; NM_020038 |
| IDO1 | NM_002164 |
| ERBB2 | NM_001005862; NM_001382784; NM_001382785; NM_001382788; NM_001382792; |
| | NM_001382793; NM_001382803; XM_047435590; NM_001289937; NM_001382786; |
| | NM_001382800; NM_001382802; NM_001382806; NM_001382782; NM_001382789; |
| | NM_001382795; NM_001289936; NM_001382797; NM_001382805; NM_004448; |
| | NR_110535; NM_001289938; NM_001382791; NM_001382801; NM_001382783; |
| | NM_001382790; NM_001382794; NM_001382798; NM_001382799; NM_001382787; |
| | NM_001382796; NM_001382804 |
| HDAC1 | XM_011541309; NM_004964 |
| RAD50 | NM_005732; NM_133482 |
| SMO | NM_005631; XM_047420759 |
| STAT6 | NM_001178078; NM_001178080; NM_001178081; XM_047429475; NM_001178079; |
| | XM_047429476; XM_047429473; XM_047429477; NM_003153; XM_047429474; |
| | NR_033659 |
| PIK3CA | NM_006218; XM_006713658 |
| HDAC7 | NR_160436; NM_015401; XM_011538481; XM_024449018; XM_047428978; |
| | NM_001308090; NM_016596; XM_011538483; XM_047428981; NR_160435; |
| | XM_047428979; XM_047428984; XM_011538480; XM_047428980; XM_047428982; |
| | XM_047428983; NM_001098416; NM_001368046 |
| IGF1R | XM_047432444; XM_011521517; NM_000875; XM_011521516; XM_017022137; |
| | XM_047432442; NM_152452; XM_047432443; XM_047432445; NM_001291858 |
| IGF1 | XM_017019263; XM_017019261; XM_017019262; XM_017019259; NM_001111284; |
| | NM_001111285; NM_001111283; NM_000618 |
| ICAM1 | NM_000201 |
| ROS1 | XM_011536053; XM_011536055; XM_011536054; XM_011536057; XM_011536049; |
| | XM_011536058; NM_001378891; XM_047419232; XM_006715548; NM_002944; |
| | XM_011536050; XM_017011173; XM_047419231; XM_011536051; XM_011536056; |
| | XM_017011172; NM_001378902 |
| MCL1 | NM_001197320; NM_182763; NM_021960 |
| TACSTD2 | NM_002353 |
| NRAS | NM_002524 |
| CCND1 | NM_053056 |
| XRCC3 | XM_005268046; NM_001371231; XM_047431767; XM_047431768; NM_001100119; |
| | NM_001371229; XM_047431766; NM_001371232; NM_001100118; NM_005432 |
| MKI67 | NM_002417; NM_001145966; XM_006717864; XM_011539818 |
| EPHA2 | XM_017000537; XM_047448267; XM_047448259; NM_001329090; XM_047448272; |
| | NM_004431 |
| BCL6 | NM_001130845; XM_011513062; NM_001706; XM_047448655; NM_001134738; |
| | NM_138931; XM_005247694 |
| BCL2L1 | XM_047440353; NM_001317919; NM_001322240; NM_001322242; XM_011528964; |
| | XM_047440351; NM_001191; NM_001317920; NR_134257; XM_017027993; |
| | NM_001317921; NM_138578; XM_047440352; NM_001322239 |
| ATF3 | XM_047421211; NM_001206488; NM_001674; NM_001206484; NM_004024; |
| | XM_005273146; NM_001040619; NM_001206486; NM_001030287; XM_011509579; |
| | NM_001206485 |
| MAGEA12 | NM_001166386; NM_001166387; NM_005367 |
| FGFR3 | XM_047449823; XM_047449824; XM_006713869; XM_006713873; NM_022965; |
| | XM_006713868; NM_001354810; XM_011513422; XM_047449821; XM_047449822; |
| | NM_000142; XM_011513420; XM_047449820; XM_006713870; XM_006713871; |
| | NM_001163213; NM_001354809; NR_148971 |
| DLL3 | NM_016941; NM_203486 |
| AREG | NM_001657 |
| PMEL | NM_001200054; NM_001200053; NM_001320121; NM_001384361; NM_001320122; |
| | NM_006928 |
| PDCD1LG2 | XM_005251600; NM_025239 |
| TPBG | NM_001166392; NM_001376922; NM_006670 |
| ATM | XM_011542844; XM_047426976; XM_047426978; NM_001351834; XM_011542840; |
| | XM_011542842; XM_047426975; NM_138293; XM_005271562; XM_006718843; |
| | XM_047426979; NM_000051; NM_001351835; XM_006718845; XM_047426981; |
| | NM_001351836; XM_011542843; XM_017017790; XM_047426977; NM_138292 |
| PIK3CG | XM_017012328; XM_005250443; XM_047420479; NM_001282426; XM_011516317; |
| | XM_047420481; XM_047420480; NM_001282427; XM_011516316; NM_002649 |
| RRM1 | NM_001033; NM_001330193; NM_001318065; NM_001318064 |
| INSR | NM_001079817; NM_000208; XM_011527989; XM_011527988 |
| CDH1 | NM_001317186; NM_004360; NM_001317185; NM_001317184 |
| KMT2C | NM_170606; NM_021230 |
| CA9 | XM_047423849; NM_001216; XM_047423850 |
| IGF2R | NM_000876 |
| CD274 | XM_047423262; NM_001314029; NM_001267706; NR_052005; NM_014143 |
| ADORA2B | XM_017024197; XM_011523661; XM_047435375; NM_000676; XM_047435374; |
| | XM_011523659; XM_047435373 |
| BIRC5 | NM_001168; NM_001012270; NM_001012271 |
| TYMS | NM_001354867; NM_001354868; XM_024451242; NM_001071 |
| MUC1 | NM_001018017; NM_001044391; NM_001044393; NM_001204291; NM_001044390; |
| | NM_001204285; NM_182741; NM_001371720; NM_001204289; NM_001204290; |
| | NM_001204293; NM_001018016; NM_001044392; NM_001204286; NM_001204287; |
| | NM_001204288; NM_001204295; NM_001018021; NM_001204292; NM_001204294; |
| | NM_001204297; NM_001204296; NM_002456 |
| MYB | NM_001161660; NR_134958; NM_001130172; NM_001130173; NM_001161656; |
| | NR_134959; NM_001161657; XM_047418834; NR_134963; NR_134965; NR_134962; |
| | XR_942444; NM_001161659; NR_134961; NM_001161658; NM_005375; NR_134960; |
| | NR_134964 |
| CCND3 | XM_047419491; NM_001287434; NM_001136017; NM_001760; NM_001136125; |
| | NM_001136126; XM_011514971; NM_001287427 |
| RB1 | NM_000321 |
| TOP1 | NM_003286 |
| MMP2 | NM_001302509; NM_001127891; NM_001302508; NM_001302510; NM_004530 |
| PTEN | NM_000314; NM_001304718; NM_001304717 |
| FN1 | NM_001306129; NM_001365519; NM_212474; NM_001306132; NM_001365517; |
| | NM_001365522; NM_001306131; NM_001365521; NM_212476; NM_212478; |
| | NM_212475; NM_001365523; NM_001365524; NM_002026; NM_001365520; |
| | NM_212482; NM_001365518; NM_054034; NM_001306130 |
| BRAF | XM_047420766; XM_047420768; NM_001374244; NM_001374258; NM_001378471; |
| | NM_001378473; NR_148928; XM_047420767; XM_047420769; XM_047420770; |
| | NM_001378467; NM_001378468; XM_017012559; NM_001378470; NM_001378472; |
| | NM_001378475; NM_001354609; NM_001378469; NM_001378474; NM_004333 |
| KMT2E | XM_047420611; NM_018682; XM_005250493; NM_032187; XM_047420613; |
| | XM_011516400; XM_047420612; NM_182931 |
| FGFR4 | NM_213647; NM_022963; NM_002011; NM_001291980; NM_001354984 |
| BRCA1 | NM_007299; NM_007303; NM_007294; NM_007306; NM_007298; NM_007295; |
| | NM_007301; NM_007300; NR_027676; NM_007305; NM_007296; NM_007297; |
| | NM_007302 |
| ERBB3 | XM_047428500; NM_001005915; XM_047428501; NM_001982 |
| CEACAM6 | NM_002483; XM_011526990 |
| EPCAM | NM_002354 |
| SMARCA4 | XM_024451667; NM_001128845; NM_001387283; NR_164683; XM_047439249; |
| | NM_001128848; XM_047439243; XM_047439246; XM_047439247; XM_047439251; |
| | XM_006722846; XM_024451661; XM_047439245; NM_001374457; XM_047439250; |
| | NM_001128846; XM_011528198; XM_024451663; NM_001128847; XM_047439244; |
| | NM_001128844; NM_001128849; NM_003072; XM_024451658; XM_047439248 |
| BRCA2 | NM_000059 |
| MTOR | NM_001386501; XM_017000900; XM_011541166; NM_001386500; XR_007058581; |
| | XM_047416721; XM_047416724; NM_004958 |
| CDK2 | NM_001290230; XM_011537732; NM_052827; NM_001798 |
| PTK7 | NM_152880; NM_152882; NM_152881; XM_047419157; NM_002821; NR_072997; |
| | NR_072998; NM_152883; NM_001270398; XM_011514766; XM_011514765 |
| EGFR | XM_047419953; NM_001346899; NM_201282; XM_047419952; NM_201284; |
| | NM_001346898; NM_001346900; NM_001346897; NM_201283; NM_001346941; |
| | NM_005228 |
| STMN1 | NM_203399; NM_203401; NM_152497; NM_005563; NM_001145454 |
| ADORA1 | NM_001048230; XM_047446499; NM_000674; NM_001365065; NM_001365066 |
| NAE1 | XM_047434835; NM_001018160; NM_003905; NM_001286500; NM_001018159 |
| IGF2 | NM_001291862; NM_001291861; NM_000612; NM_001007139; NM_001127598 |
| IRF2 | NM_002199 |
| ABCB1 | NM_001348946; NM_001348944; NM_000927; NM_001348945 |
| WT1 | NM_000378; NR_160306; NM_001367854; NM_001198551; NM_001198552; |
| | NM_024424; NM_024426; NM_024425 |
| MDM2 | NM_006880; NM_006882; XM_047428853; NM_006878; NM_001145340; |
| | NM_001278462; NM_001367990; NM_006879; NM_001145337; NM_002392; |
| | NM_006881; NM_032739; NM_001145339; NM_001145336 |
| MAGEA10 | NM_001251828; NM_021048; NM_001011543 |
| ERCC1 | NM_001369419; NM_001369409; NM_001166049; NM_001369412; NM_001369417; |
| | NM_202001; NM_001369415; NM_001369418; NM_001369408; NM_001369410; |
| | NM_001369411; NM_001369413; NM_001369414; NM_001369416; NM_001983 |
| ADORA2A | NM_000675; NR_103544; NM_001278498; NM_001278499; NM_001278500; NR_103543; |
| | NM_001278497 |
| KRAS | XM_047428826; NM_001369786; NM_033360; NM_004985; NM_001369787 |
| ITGB4 | XM_047435927; XM_005257311; XM_006721866; XM_006721870; NM_000213; |
| | NM_001005619; NM_001005731; XM_005257309; XM_011524752; XM_006721867; |
| | XM_011524751; XM_047435929; NM_001321123; XM_047435926; XM_047435928; |
| | XM_006721868 |

TABLE 2
Genes Associated with TME Cells

| | |
|---|---|
| CD74 | NM_001364083; NM_001364084; NR_157074; NM_001025159; NM_001025158; |
| | NM_004355 |
| HPR | NM_001384360; XM_024450251; NM_020995 |
| TNFRSF4 | XM_011542074; NM_003327; XR_007063145; XM_011542077; XM_011542075; |
| | XM_011542076 |
| SERPINF1 | XR_004837577; NM_001329904; NM_001329905; NM_002615; NM_001329903 |
| FAM26F | NM_001010919; NM_001276460; XM_011535845 |
| PPP3CC | XM_047421941; XM_047421942; NM_001243975; NM_005605; XR_007060744; |
| | NM_001243974 |
| DEFA3 | XM_011534741; NM_005217 |
| GZMB | NM_001346011; NM_004131; NR_144343 |
| GNG8 | NM_001198756; NM_001198754; NM_001198755; NM_031498 |
| FCGR3A | XM_047449443; NM_001127595; NM_001329122; XM_047449444; NM_001127596; |
| | NM_001127592; NM_000569; NM_001386450; NM_001127593; NM_001329120 |
| CISH | NM_013324; XM_047447398; NM_145071 |
| NFKBIA | NM_020529 |
| C1QA | NM_001347466; NM_001347465; NM_015991 |
| CD8A | NM_001382698; NM_001145873; NM_001768; NR_168478; NR_168479; NM_171827; |
| | NR_168480; NR_168481; NR_027353 |
| CSF3R | NM_000760; XM_005270493; NM_156039; XM_011540749; NM_156038; NM_172313; |
| | XM_047446753 |
| LTB | NM_002341; NM_009588 |
| NCR3 | NM_001145467; XM_011514459; XM_006715049; NM_001145466; NM_147130 |
| PAX5 | NM_001280547; NM_001280553; NM_016734; NM_001280548; NR_103999; |
| | NM_001280551; NM_001280555; NM_001280554; NM_001280552; NM_001280556; |
| | NM_001280550; NM_001280549; NR_104000 |
| ITGAL | XM_005255313; XM_006721044; NM_001114380; XR_950794; XM_047434073; |
| | XM_047434072; NM_002209 |
| PTGDR | XM_005267891; NM_000953; NM_001281469 |
| FFAR2 | XM_047438699; NM_005306; NM_001370087; XM_017026711; XM_047438700 |
| KIR2DL1 | NM_014218 |
| STAP1 | NM_001317769; NM_012108 |
| EGR2 | NM_001321037; NM_001136179; NM_001136177; NM_001136178; XM_011539427; |
| | NM_000399 |
| SH2D1A | NM_001114937; NM_002351 |
| DOK2 | NM_001401272; NM_001317800; NM_201349; NM_003974 |
| HLA-DRB3 | NM_022555 |
| CLEC5A | XR_007059995; XM_011515995; NM_001301167; NM_013252 |
| CCL13 | NM_005408 |
| MYO1G | XR_007060129; NM_033054 |
| PRKCB | NM_212535; NM_002738; XM_047434365 |
| ATP2A3 | XM_011523881; XM_011523882; XM_011523884; XM_011523888; XM_011523892; |
| | XM_047436152; NM_174957; XM_047436151; XM_047436153; NM_005173; |
| | NM_174954; NM_174958; XM_011523889; NM_174955; XM_011523885; NM_174956; |
| | XM_047436150; NM_174953 |
| AMFR | XM_005255890; NM_001144; NM_001323512; NM_138958; NM_001323511 |
| LRRN3 | NM_018334; NM_001099660; NM_001099658 |
| IL18RAP | NM_001393489; XM_047446162; XM_011512088; XM_024453197; NM_001393487; |
| | NM_001393486; XR_007083519; XM_024453199; XM_024453201; NM_003853; |
| | XM_024453198; XM_047446163; NM_001393488 |
| FCRL6 | XM_011509480; XM_047419607; NM_001004310; XM_011509481; XM_047419606; |
| | NM_001284217; XM_005245128; XM_005245129; XM_005245131 |
| LYVE1 | NM_006691 |
| SIGLEC14 | XM_047437991; NM_001098612 |
| CD248 | NM_020404 |
| FGL2 | NM_006682 |
| STK4 | NM_001352385; XM_017028033; XM_011529018; XM_017028031; NM_006282; |
| | NR_147974; NR_147975; XM_005260532; XM_047440425; XM_047440426 |
| FCRLA | NM_032738.4; NM_001184866.2; NM_001184867.2; NM_001184870.2; NM_001184871.2; |
| | NM_001184872.2; NM_001184873.2; NM_001366195.2; NM_001366196.2 |
| IRF4 | NM_002460.4; NM_001195286.2; NR_046000.3 |
| SIRPG | XM_011529286; NM_018556; XM_011529287; XM_005260749; NM_001039508; |
| | NM_080816 |
| MRC1 | NM_002438; NM_001009567 |
| LILRB4 | NM_001278429; NM_001394939; NM_001394934; NM_006847; NM_001278428; |
| | XM_017026216; XM_047438100; NM_001394935; NM_001081438; XM_047438102; |
| | XM_047438103; NM_001394938; XM_047438101; NM_001278426; NM_001394933; |
| | NM_001394937; NM_001278427; NM_001278430; NM_001394936 |
| MPEG1 | NM_001039396 |
| CD80 | NM_005191 |
| NR4A3 | NM_173200; NM_006981; NM_173199; NM_173198; XM_017015162 |
| HHIP | XM_005263178; NM_022475; XM_006714288 |
| PARP15 | XM_011512476; XM_005247160; XM_005247159; XM_017005791; XM_017005792; |
| | XM_047447580; XM_047447584; XM_011512475; NM_001113523; XM_047447582; |
| | NM_001308320; XM_011512480; XM_011512477; XM_011512479; XM_047447583; |
| | NM_001308321; NM_152615 |
| CD247 | NM_001378516; NM_198053; XM_011510144; XM_011510145; NM_000734; |
| | NM_001378515 |
| RASGRP1 | XM_047432077; NM_001128602; NM_005739; XM_047432073; XM_047432076; |
| | XM_047432078; XM_047432074; NM_001306086; XM_047432075 |
| GLT1D1 | NR_159493; NM_144669; XM_047428373; XM_047428371; XM_047428372; |
| | XR_001748588; XM_011537957; NM_001366886; NM_001366887; NM_001366888; |
| | NM_001366889; NR_133646 |
| SOD2 | NM_001322817; NM_001322820; NM_001322815; NM_001322814; NM_000636; |
| | NM_001322816; NM_001024465; NM_001024466; NM_001322819 |
| JCHAIN | NM_144646 |
| CD38 | NM_001775; NR_132660 |
| IGHM | NG_001019.6 |
| PDCD1 | NM_005018; XM_006712573 |
| LYZ | NM_000239 |
| LY86 | NM_004271 |
| PIK3AP1 | XM_005269499; XM_047424566; NM_152309; XM_011539248 |
| SLC15A3 | XM_011545095; NR_027391; XR_007062485; NM_016582 |
| IL27 | NM_145659 |
| CD300E | NM_181449 |
| CD37 | XM_005259435; XM_011527542; NM_001774; XM_011527543; NM_001040031 |
| COL1A1 | XM_005257058; XM_005257059; XM_011524341; NM_000088 |
| TRAC | NG_001332.3 |
| ARHGAP25 | XM_017005426; XM_011533210; NM_001007231; NM_001166276; NM_001364819; |
| | NM_001166277; NM_001364820; NM_014882; XM_011533207; XM_011533209; |
| | NM_001364821 |
| GRAP2 | NM_001291825; NM_001291826; XM_047441608; NM_001291824; XM_047441607; |
| | NM_004810; XR_007067996; NM_001291828; XR_007067995 |
| CCR4 | XM_017005687; NM_005508 |
| RUNX3 | NM_001031680; NM_004350; XM_011542351; XM_005246024; XM_047433131; |
| | NM_001320672 |
| XCL1 | NM_002995 |
| C1QC | NM_001114101; NM_001347619; NM_001347620; NM_172369 |
| MMP25 | NM_024302; XM_011525227; NM_001032278; NM_032950; XM_011525225; |
| | XM_011525230; XM_024450943; XM_011525226; NR_111988; XM_011525229; |
| | XM_011525231; XM_011525232; XM_017025063; XM_017025064; XM_047436731 |
| SPOCK2 | NM_001244950.2; NM_014767.2; NM_001134434.1 |
| IL17F | NM_052872.4; XM_011514276.1 |
| CD28 | NM_006139.4; NM_001243077.2; NM_001243078.2 |
| TNFRSF13C | XM_011514276; NM_052872; NM_172343 |
| PVRIG | NM_006139; NM_001243078; NM_001243077; XM_011512194 |
| SH2D1B | NM_052945 |
| AOAH | NM_024070; NM_001397246; NM_001387134 |
| NCF4 | NM_053282 |
| FCMR | NM_001177507; XM_011515335; XM_011515341; XM_011515336; XM_011515340; |
| | XM_011515342; XM_017012105; XM_011515333; XM_011515334; XM_047420297; |
| | NM_001177506; NM_001637; XM_011515338; XM_011515339; XM_017012104; |
| | XM_017012102 |
| TAGAP | XM_047441385; NM_000631; XM_047441384; NM_013416 |
| ITK | XM_047434335; NM_001193338; NM_005449; NM_001142473; XM_047434334; |
| | XM_047434331; NM_001142472; XM_005273351 |
| SPI1 | NM_001278733; NM_138810; NM_054114; NM_152133 |
| CD244 | NM_005546 |
| ITGB2 | XM_017018173; NM_003120; XM_047427487; NM_001080547 |
| TRAF3IP3 | NM_001166663; XM_011509622; XM_047422535; NM_016382; NM_001166664; |
| | XM_011509623; XM_011509621 |
| LAPTM5 | XM_047440763; NM_000211; NM_001303238; XM_006724001; NM_001127491 |
| CD79A | NM_025228; NR_109871; XM_047430963; NM_001287754; NM_001320143; |
| | XM_005273279; XM_047430964; XM_011510018; XM_017002400; XM_011510019; |
| | NM_001320144; XM_047430976 |
| SLAMF6 | XM_011542098; NM_006762 |
| SLA2 | NM_021601; NM_001783 |
| CD8B | NM_001184714; XM_047443866; NM_001184715; NM_052931; NM_001184716; |
| | XM_017000216 |
| CD96 | NM_175077; NM_032214 |
| SERPINB9 | NM_172102; NM_172100; NM_001178100; NM_004931; NM_172101; NM_172213; |
| | NM_172099; XM_011533164 |
| FGR | XR_007093316; NR_134917; XR_007093335; XR_241462; XM_006713470; NM_005816; |
| | XR_924090; XM_006713469; NM_198196; XR_007093273; XR_007093326; |
| | XR_007093307; XM_047447184; NM_001318889; XR_001739977; XR_007093366 |
| KLRG1 | XM_005249184; NM_004155; XM_011514678; XM_047418894 |
| HAVCR2 | NM_005248; NM_001042729; NM_001042747 |
| RASAL3 | XM_017018682; XM_017018684; XM_047428074; NR_137426; NM_001329102; |
| | NR_137427; NM_001329103; NM_001329099; NM_001329101; NM_005810; NR_137428; |
| | XM_017018685; XM_047428075 |
| PARP8 | NM_032782 |
| CTLA4 | XM_047439231; NR_174477; NR_174478; XM_011528187; NM_001400377; |
| | XM_011528186; NM_001400378; NM_001400381; NM_022904; NM_001348027; |
| | NM_001348028; NM_001400379; NM_001400380 |
| BLK | XM_011543632; XM_011543634; XM_011543631; XM_047417705; XM_047417708; |
| | NM_001178056; XM_011543643; XM_005248596; NM_001331028; XM_047417707; |
| | NM_001178055; XM_011543633; XM_047417706; NM_024615 |
| PILRA | NM_001037631; NM_005214 |
| FCRL3 | XM_047422081; NM_001330465; XM_011543829; XM_011543824; XM_011543827; |
| | XM_047422083; XM_047422084; XM_011543828; XM_047422082; NM_001715; |
| | XM_011543825 |
| DUSP2 | XM_047420291; NM_178273; NM_178272; NM_013439; XM_047420292 |
| CXCL10 | NR_135216; NR_135217; XM_006711145; NM_001320333; NM_052939; NR_135214; |
| | NR_135215; NM_001024667 |
| IL1B | XM_017003546; NM_004418 |
| DPEP2 | NM_001565; NR_168520 |
| HLA-DPB1 | NM_000576; XM_047444175 |
| SAMSN1 | XM_011523273; XM_047434462; XM_047434464; NR_136706; XM_011523271; |
| | XM_005256090; XM_024450376; NM_022355; XM_047434463; XM_011523266; |
| | XM_024450372; XM_024450373; XM_024450374; XM_047434459; XM_047434465; |
| | XM_011523268; XM_011523274; XM_017023547; NM_001324159; XM_017023545; |
| | XM_047434460; XM_047434461; NM_001369657; XR_243420; XR_933392 |
| RASSF5 | NM_002121 |
| CCL18 | XM_011529684; NM_001256370; NM_001395858; XM_047440942; NM_001286523; |
| | NM_022136; XM_047440941; XM_011529685; XM_011529686; NM_001256579; |
| | NM_001395856; NM_001395857 |
| TYROBP | NM_182663; NM_031437; NM_182664; NM_182665 |
| KLRC2 | NM_002988 |
| MAP4K1 | NM_001173515; NM_003332; NR_033390; NM_001173514; NM_198125 |
| PIM2 | NM_002260 |
| CST7 | XM_011526404; NM_001042600; NM_007181 |
| TESPA1 | NM_006875; XM_047441792 |
| SNX20 | NM_003650 |
| CD300A | XM_006719715; XM_047429930; NM_001136030; NR_147068; XR_007063147; |
| | XM_011539035; NR_147064; NR_147065; NR_147072; NR_147073; XM_017020262; |
| | XM_047429929; NM_001261844; NM_001351152; NR_147066; NR_147071; |
| | XM_017020263; NM_001351149; NR_147069; XM_011539037; NM_001098815; |
| | NM_001351151; NM_014796; NR_147067; NM_001351150; NM_001351154; |
| | NM_001351155; NR_147062; NR_147063; NR_147070; XM_047429931; NM_001351148; |
| | NM_001351153; XR_007063146 |
| TBC1D10C | NM_001144972; NM_153337; NM_182854 |
| GZMK | XM_005256991; NM_001330457; NM_001330456; XM_005256990; NM_007261; |
| | NM_001256841 |
| AKNA | XM_011545002; NM_001369495; XM_047426913; NM_001369492; NM_001256508; |
| | NM_001369494; NM_198517; XM_006718539; XM_047426910; NM_001369498; |
| | XM_006718538; XM_047426911; XM_047426914; NM_001369496; NR_046266; |
| | XM_047426909; NM_001369497 |
| COL3A1 | NM_002104 |
| CLEC2D | XM_005252247; XR_929844; XM_011519063; NM_001317950; NM_001317952; |
| | XM_011519065; XM_047423926; XM_047423924; XM_011519066; XM_047423921; |
| | XM_047423922; XM_047423925; XM_005252245; XM_005252248; XM_006717294; |
| | XM_047423923; NM_030767; XM_011519064; XM_005252244 |
| PLCB2 | NM_000090; NM_001376916 |
| PRDM1 | NM_001197318; NM_001004420; NM_013269; NR_036693; NM_001197319; |
| | NM_001197317; NM_001004419 |
| TNFRSF1B | XM_047432672; XM_047432683; XM_017022317; XM_047432676; XM_047432679; |
| | NM_004573; XR_007064458; XM_017022314; XM_047432670; NM_001284297; |
| | XM_047432678; XM_047432681; NM_001284298; NM_001284299; XM_047432669; |
| | XM_047432671; XM_047432673; XM_047432674; XM_047432677; XM_047432682; |
| | XM_047432689; XM_017022319; XM_047432675; XM_047432684; XM_047432686; |
| | XM_047432667; XM_047432668; XM_047432680; XM_047432685; XM_047432687; |
| | XM_047432688 |
| IGHD | XM_047419248; XM_047419247; XM_011536064; XM_017011187; XM_011536062; |
| | XM_047419246; XM_006715550; NM_182907; NM_001198 |
| TNFAIP6 | XM_047429422; NM_001066; XM_047429424; XM_011542060; XM_011542063; |
| | XM_047429423 |
| KLRB1 | NM_002258 |
| CD69 | NR_026672; NR_026671; NM_001781 |
| CD5 | NM_014207; NM_001346456 |
| FPR2 | NM_001005738; NM_001462; XM_006723120 |
| KIR3DL2 | XM_047438795; NM_006737; NM_001242867 |
| CCL4L2 | NM_001291475.2; NM_001291468.2; NM_001291469.2; NM_001291470.2; |
| | NM_001291471.2; NM_001291472.2; NM_001291473.2; NM_001291474.2; NR_111970.2 |
| CD3D | NM_000732.6; NM_001040651.2 |
| ACSL1 | NM_001995.5; NM_001286708.2; NM_001286710.2; NM_001286711.2; NM_001381877.1; |
| | NM_001381878.1; NM_001381879.1; NM_001381880.1; NM_001381881.1; |
| | NM_001381882.1; NM_001381883.1; NM_001381884.1; NM_001381885.1; |
| | NM_001381886.1; NM_001381887.1; NM_001381888.1; NM_001381889.1; |
| | NM_001381890.1; NR_167698.1; NR_167702.1 |
| PECAM1 | XM_047436251; NM_000442; XM_005276883; XM_017024741; XM_017024739; |
| | XM_005276880; XM_005276881; XM_005276882 |
| RCSD1 | NR_136519; NM_052862; NM_001322923; NM_001322924 |
| VWF | NM_000552; XM_047429501 |
| HCK | NM_001172132; NM_001172133; NM_001172130; NM_002110; NM_001172131; |
| | NM_001172129 |
| NR4A2 | XM_011511246; NM_173171; XM_005246621; XM_047444551; NM_173172; |
| | XM_047444557; XM_047444558; XM_047444559; NM_173173; XM_006712553; |
| | NM_006186; XM_047444555; XM_047444554 |
| C3AR1 | NM_004054; NM_001326475; NM_001326477 |
| PIK3IP1 | NM_001135911; NM_052880 |
| GK | NM_203391; NR_174372; NR_174371; XM_006724483; NR_174374; NR_174375; |
| | NR_174370; XM_011545491; XM_011545492; NM_000167; NR_174369; NR_174373; |
| | NM_001128127; NM_001205019; NM_001399987 |
| NOS3 | NM_001160110; NM_000603; NM_001160109; NM_001160111 |
| PLEKHO2 | NM_001098622; NR_146096; NR_146095; NR_146097 |
| PIK3R5 | NM_025201; NM_001195059 |
| SP140 | XM_017003249; XM_047443078; XM_011510515; XM_011510516; XM_011510517; |
| | XM_017003250; XM_017003253; XM_047443073; XM_047443076; XM_047443077; |
| | NM_001278452; NM_001278453; XM_011510520; XM_017003245; XM_017003246; |
| | XM_017003252; XM_047443074; XM_005246253; XM_005246255; XM_011510518; |
| | XM_017003247; XM_005246252; XM_005246256; XM_017003248; XM_047443079; |
| | XM_047443080; NM_001278451; XM_017003242; XM_005246254; XM_006712223; |
| | XM_017003240; XM_017003243; XM_047443072; XM_04744308 1; NM_007237; |
| | XM_011510519; XM_017003239; NM_001005176 |
| KLRF1 | XM_017019415; XM_047428956; NM_001291822; NM_001366534; NR_120305; |
| | NM_001291823; NM_016523; NR_159359; NR_159360; NR_159361 |
| MS4A7 | NM_021201; NM_206940; NM_206939; NM_206938 |
| PTPRCAP | NM_005608 |
| CREM | XM_011519331; XM_011519333; XM_047424626; XM_047424627; XM_047424630; |
| | XM_047424632; XM_047424637; NM_001352445; NM_001352446; NM_001394625; |
| | NM_182720; NM_182770; NM_183013; XM_047424635; NM_001267569; |
| | NM_001352465; NM_001394595; NM_001394614; NM_001394626; NM_181571; |
| | NM_182723; NM_183012; XM_047424634; NM_001267564; NM_001394598; |
| | NM_001394613; NM_001394616; NM_001394617; NM_001394621; NM_001394627; |
| | XM_047424625; XM_047424633; NM_001267563; NM_001394619; NM_001394630; |
| | NM_001394631; NM_182718; NM_182721; NM_182769; NR_172139; XM_011519325; |
| | XM_011519332; XM_017015731; XM_047424636; NM_001394602; NM_001394603; |
| | NM_001394618; NM_001394622; NM_001881; XM_011519324; XM_024447824; |
| | NM_001267562; NM_001267566; NM_001352466; NM_001394608; NM_001394628; |
| | NM_001394629; NM_182719; NM_182724; NM_182772; NM_182725; NM_182850; |
| | XM_006717382; XM_011519335; XM_047424628; NM_001267568; NM_001267570; |
| | NM_001394605; NM_001394610; NM_001394615; NM_183011; NM_183060; |
| | NM_182853; XM_006717387; XM_011519330; XM_047424629; XM_047424631; |
| | NM_001267565; NM_001267567; NM_001352467; NM_001394600; NM_001394620; |
| | NM_001394623; NM_182717; NM_182771; NR_172138; NM_182722 |
| FERMT3 | NM_001382362; NM_001382363; NM_001382364; NM_001382448; NM_031471; |
| | XM_047427676; NM_001382361; NM_178443 |
| ITGA4 | NM_001316312; NM_000885 |
| CORO1A | NM_007074; NM_001193333 |
| CLEC7A | NM_022570; NM_197948; NM_197951; NM_197953; XM_047429359; XM_047429360; |
| | NM_197947; NM_197954; NM_197952; NM_197950; NR_125336; XM_024449132; |
| | NM_197949; XM_006719135; XM_024449133 |
| MSR1 | NM_138716; NM_002445; XM_024447161; NM_138715; NM_001363744 |
| TNFRSF17 | NM_001192 |
| S100A12 | NM_005621 |
| ARHGAP15 | NM_018460; XM_011511482; XM_024453000; XM_011511483; XM_017004500; |
| | XM_047445110; XM_047445112; XR_007078554; XM_011511484; XM_047445109; |
| | XM_047445 ill; XM_047445114; XM_047445113 |
| MS4A6A | XM_011545209; NM_001330275; NM_022349; NM_152851; XM_005274177; |
| | XM_017018125; XM_047427403; NM_001247999; XM_047427402; NM_152852; |
| | XM_024448652; XM_006718660; XM_006718661 |
| PARVG | NM_001254742; NM_022141; NM_001137605; NM_001254743; XM_047441455; |
| | NM_001254741; NM_001137606 |
| CCL22 | XM_047434450; XM_047434449; NM_002990 |
| ABI3 | NM_016428; XM_005257429; XM_011524873; XM_017024721; NM_001135186 |
| PTPN22 | XM_011541225; NM_001193431; XM_047417632; XM_011541223; XM_017001006; |
| | NM_015967; XM_011541221; XM_011541222; NM_012411; XM_017001005; |
| | XM_047417630; XM_047417631; NM_001308297 |
| FPR1 | NM_002029; NM_001193306 |
| NCR1 | NM_004829; NM_001145457; XM_011527530; XM_047439727; NM_001242357; |
| | XM_011527529; NM_001242356; NM_001145458 |
| CCRL2 | NM_003965; XM_011534208; NM_001130910 |
| FCRL1 | NM_001184867; NM_001184870; NM_001184866; NM_032738; XM_006711581; |
| | XM_011510065; NM_001184873; NM_001184871; NM_001184872; NM_001366195; |
| | NM_001366196 |
| CSRNP1 | NM_001320560; NM_033027; NM_001320559; XM_047448721; XM_047448723; |
| | XM_047448724; XM_017007049 |
| CSF1R | NM_001375320; NM_005211; NR_164679; NM_001349736; NM_001288705; |
| | NM_001375321; NR_109969 |
| P2RY10 | NM_001324221; NM_001324225; NM_014499; NM_001324218; NM_198333; |
| | XM_047441998 |
| GPR171 | XM_047448056; XM_005247402; NM_013308; XM_047448055; XM_047448054; |
| | XM_005247403 |
| GNG2 | XM_017021377; NM_001389707; NM_001243773; NM_001389709; XM_047431485; |
| | XM_024449634; XM_047431486; XM_047431487; XM_047431488; XM_047431490; |
| | NM_001389708; NM_001243774; NM_001389710; XM_024449633; NM_053064 |
| CCR7 | NM_001301716; NM_001301717; NM_001838; NM_001301718; NM_001301714 |
| CCL7 | NM_006273 |
| ESM1 | NM_001135604; NM_007036 |
| EMCN | NM_001159694; XM_017008290; NM_016242; XM_011532024 |
| TNFRSF10C | NM_003841 |
| ACTA2 | NM_001141945; NM_001320855; NM_001613 |
| CECR1 | XM_047441407; NM_001282228; NM_017424; XM_047441406; NM_001282225; |
| | NM_001282227; NM_177405; XM_011546133; NM_001282226; NM_001282229 |
| HK3 | XM_047417134; XM_011534540; NM_002115; XR_941102 |
| HLA-DRB5 | XM_011514562; NM_002125 |
| CSF2RB | XM_011529904; XM_005261340; XM_047441149; XM_011529903; XM_047441150; |
| | XM_047441148; NM_000395 |
| ECSCR | NM_001077693; NM_001293739; NR_121659 |
| KIR3DL1 | XM_017030274; NM_001322168; NM_013289 |
| IL4I1 | NM_001385639; NM_172374; NM_152899; NR_047577; NM_001258018; NM_001258017 |
| MEFV | NM_001198536; NM_000243 |
| SELL | NR_029467; NM_000655 |
| LRMP | XM_047428841; NM_001366540; NM_001366546; NM_001204126; NM_001366542; |
| | NM_006152; NM_001204127; NM_001366545; NR_159369; XM_047428842; |
| | NM_001366544; NR_159366; NM_001321724; NM_001366541; NM_001366548; |
| | NM_001366549; NM_001366543; NM_001366547; NM_001394803; XM_047428840; |
| | NR_159367; NR_159368 |
| ABTB1 | XM_006713769; NR_033429; NM_172028; NM_032548; XM_017007285; |
| | XM_017007286; NM_172027 |
| IL23A | NM_016584 |
| LST1 | NM_205838; NM_001166538; NR_029461; NM_205839; XM_006715209; |
| | XM_006715210; NM_205837; XM_006715206; XM_047419357; NM_007161; |
| | XM_011514914; NR_029462; NM_205840 |
| TNFRSF18 | NM_148901; NM_004195; XM_017002722; NM_148902 |
| AIF1 | NM_001318970; NM_032955; NM_004847; NM_001623; XM_005248870 |
| STK17B | XM_011512171; XM_047446334; XM_047446333; XM_011512170; XM_047446335; |
| | NM_004226; XM_011512169 |
| ELMO1 | XM_011515654; XM_047421091; XM_005249919; XM_047421086; XM_047421090; |
| | NM_001206480; NM_130442; XM_006715805; NM_001039459; XM_047421087; |
| | NR_038120; XM_017012839; XM_024447008; XM_047421088; NM_001206482; |
| | NM_014800; XM_047421089 |
| GPR183 | NM_004951 |
| MNDA | NM_002432 |
| C5AR1 | XM_047439300; NM_001736 |
| F13A1 | NM_000129 |
| CD3G | XM_005271724; XM_006718941; NM_000073 |
| CCL4 | NM_002984.4 |
| CD72 | XM_047424157; XM_006716893; XM_047424154; NM_001782; XM_047424155; |
| | XM_047424156 |
| CD19 | NM_001178098; NM_001385732; NM_001770; XR_950871; NR_169755; XM_011545981 |
| RHOH | XM_047415675; NM_001278361; NM_001278364; XM_017008189; NM_001278360; |
| | NM_001278363; NM_001278359; NM_001278365; NM_001278362; XM_047415674; |
| | NM_001278369; NM_001278367; NM_001278368; XM_011513692; NM_001278366; |
| | NM_004310 |
| IFNG | NM_000619 |
| TRGC2 | NG_001336.2 |
| FCGR2A | NM_001136219; NM_021642; XM_024454040; XM_017000664; XM_017000665; |
| | XM_017000663; XM_017000666; XM_047449441; XM_011509290; XM_011509291; |
| | NM_001375296; NM_001375297 |
| TTN | XM_017004820; XM_024453095; XM_024453100; NM_003319; XM_017004819; |
| | XM_024453097; XM_047445661; NM_133378; NM_133379; XM_047445663; |
| | NM_133432; NM_133437; XM_017004823; XM_024453098; XM_047445660; |
| | XM_047445668; NM_001267550; XM_017004822; XM_024453099; XM_017004821; |
| | XM_047445665; NM_001256850 |
| ICAM3 | NM_001395374; NM_001395376; NM_001320605; NM_001320606; NM_002162; |
| | NM_001395375; NM_001320608 |
| THEMIS2 | XM_047434895; NM_001105556; NM_001286113; NM_004848; XM_006711050; |
| | NM_001039477; XM_005246041; XM_011542445; NM_001286115 |
| TRDC | NG_001332.3 |
| IL16 | XM_047432448; NM_004513; NM_172217; XM_047432451; XM_047432458; |
| | NM_001172128; NM_001352684; NR_148035; XM_047432450; XM_047432457; |
| | NM_001352686; XM_047432452; NM_001352685; XM_047432447; XM_047432454; |
| | XM_047432449; XM_047432453; XM_047432455; XM_047432456 |
| TIE1 | XM_047429354; XM_005271163; NM_001253357; XM_017002207; XM_047429343; |
| | NM_005424 |
| COL1A2 | NM_000089 |
| LILRB1 | XM_017026192; NM_001081637; NM_001081639; NM_001278399; XM_047438080; |
| | XM_047438084; XM_047438085; NM_001081638; NM_006669; XM_047438081; |
| | NM_001278398; NM_001388358; XM_047438083; NM_001388355; NM_001388357; |
| | NR_103518; XM_047438082; XM_047438086; NM_001388356; XM_047438089; |
| | XM_047438087; XM_047438088 |
| BTG1 | NM_001731 |
| IGLL5 | NM_001178126; NM_001256296 |
| PDE4B | XM_047422401; NM_001297441; NM_001037341; NM_001037339; NM_002600; |
| | XM_017001445; NM_001297440; NM_001297442; XM_005270924; XM_005270925; |
| | XM_006710680; NM_001037340 |
| FCN1 | NM_002003 |
| HLA-DQB1 | NM_001243962; NM_001243961; NM_002123 |
| PHOSPHO1 | XM_047435505; NM_001143804; XM_047435504; NM_178500; XM_047435506 |
| RORA | XM_047432930; XM_011521874; XM_011521879; XM_047432929; NM_002943; |
| | XM_011521875; XM_047432928; NM_134260; NM_134261; XM_011521877; NM_134262 |
| ADGRE2 | XM_047438731; XM_011527955; XM_047438726; NM_001271052; NM_152916; |
| | XM_011527954; XM_011527953; XM_047438720; XM_047438727; NM_152918; |
| | XM_017026727; XM_047438721; XM_047438733; XM_047438736; XM_011527948; |
| | XM_011527951; XM_011527952; XM_017026726; XM_047438722; XM_047438724; |
| | NM_152919; XM_011527949; XM_047438723; XM_047438725; XM_047438729; |
| | XM_047438730; XM_047438735; XM_047438732; NM_013447; NM_152917; |
| | NM_152920; XM_047438728; XM_047438734; NM_152921 |
| CTSW | NM_001335 |
| SASH3 | NM_018990; XM_006724763 |
| FCER1G | NM_004106 |
| AC243829.1 | AK022182.1 |
| BCL2A1 | NM_004049.4; NM_001114735.2 |
| THBS2 | NM_003247.5; NM_001381939.1; NM_001381940.1; NM_001381941.1; NM_001381942.1; |
| | NR_167744.1; NR_167745.1 |
| HCST | NM_001007469; XM_017026193; XM_047438090; NM_014266 |
| HLA-DRB1 | XM_024452553; NM_001359194; XM_047444767; XM_047444769; NM_001243965; |
| | NM_002124; XM_047444770; NM_001359193; XM_047443024; XM_047444768 |
| CD27 | NM_001242; XM_011521042; XM_017020234; XM_047429900 |
| P2RY13 | XM_006713664; NM_023914; NM_176894 |
| ITM2A | NM_001171581; NM_004867 |
| APOBEC3G | NM_001349436; NM_001349437; NR_146179; NM_021822; NM_001349438 |
| HLA-DQA2 | NM_020056 |
| CD163 | XM_047429895; XM_024449278; NM_203416; NM_001370145; NM_001370146; |
| | NM_004244; NR_163255 |
| CCR1 | NM_001295 |
| CD7 | NM_006137 |
| VNN2 | XM_006715593; NR_110143; NR_110146; XM_011536231; XR_007059352; |
| | NM_001242350; XM_047419477; XM_047419480; NM_004665; NM_078488; |
| | NR_034173; NR_110144; NR_110145; XM_047419479; XM_047419481; NR_034174; |
| | XM_047419478 |
| APOA2 | NM_001643 |
| CYTIP | NM_004288; XM_017005386 |
| BANK1 | NM_001127507; NM_001083907; NM_017935 |
| CD52 | NM_001803 |
| IRF8 | XM_047434052; NM_001363908; NM_002163; NM_001363907 |
| TFEB | XM_006715212; NM_001271943; NM_001271945; NM_001167827; XM_047419361; |
| | NM_007162; NM_001271944; XM_005249411 |
| PTPN6 | XM_011520988; NM_002831; XM_047429231; XM_024449106; NM_080548; |
| | XM_047429232; NM_080549 |
| LAG3 | NM_002286; XM_047428839; XM_011520956 |
| NPL | NM_001200051; NM_001200052; NM_030769; NM_001200050; NM_001200056 |
| PREX1 | NM_020820; XM_047440333; XM_047440332; XM_047440331; XM_011528934; |
| | XM_047440334 |
| ENTPD1 | XM_017016963; NM_001164179; NM_001164181; XM_011540374; XM_047426024; |
| | NM_001164183; XM_011540371; NM_001164178; XM_011540372; XM_011540376; |
| | XM_047426027; XM_047426029; NM_001312654; XM_047426025; XM_047426026; |
| | XM_047426028; NM_001164182; NM_001776; XM_017016958; XM_017016964; |
| | XM_011540370; XM_011540373; XM_047426023; NM_001098175; NM_001320916 |
| KLRC3 | NM_002261; NM_007333 |
| TAGLN | NM_001001522; NM_003186 |
| THEMIS | XM_047418763; XM_047418766; XM_047418767; NM_001164687; XM_047418764; |
| | NM_001318531; NM_001394521; XM_047418765; NM_001164685; NM_001394520; |
| | NM_001394522; NM_001010923 |
| CD6 | XM_047427875; XM_047427876; XM_047427879; XM_011545360; XM_047427878; |
| | XM_047427881; NM_001254750; NM_001254751; NM_006725; NR_045638; |
| | XM_006718738; XM_006718739; XM_047427877; XM_006718740; XM_011545362; |
| | XM_047427874; XM_047427880 |
| ADGRE3 | NM_032571; NM_152939; XR_001753772; XM_011528374; XM_047439546; |
| | NM_001289158; NM_001289159 |
| FCGR3B | NM_001271036; NM_001271037; NM_000570; NM_001244753; NM_001271035 |
| RASGEF1B | NM_001300735; NM_001300736; NM_152545 |
| CXCR4 | NM_001348059; NM_001348060; XM_047445802; NM_001348056; NM_003467; |
| | NM_001008540 |
| MARCO | NM_006770; XM_011512082; XM_011512083; XM_017005171 |
| PLA2G7 | XM_047419360; NM_001168357; XM_005249408; NM_005084; XM_047419359 |
| GBP5 | NM_052942; NM_001134486; NM_001391920 |
| PYHIN1 | XM_005244930; NM_198930; NM_152501; NM_198928; XM_011509243; NM_198929; |
| | XM_011509242 |
| CXCL3 | NM_002090 |
| NCF2 | XM_047421222; XM_047421229; XM_047421238; NM_001190789; XM_005245207; |
| | XM_047421231; NM_001127651; NM_001190794; NM_000433; XM_011509580; |
| | XM_011509581 |
| CD48 | XM_017002867; XM_047435011; NM_001778; XM_005245625; NM_001256030 |
| INPP5D | XM_047444219; NM_005541; NM_001017915; XM_047444220 |
| SLAMF7 | XM_011509828; XM_011509829; NM_001282589; NM_001282590; NM_001282596; |
| | NM_001282591; NM_001282593; NM_001282588; NM_001282595; XM_047426359; |
| | NM_001282592; NM_001282594; NM_021181 |
| ANKRD44 | XM_047446282; NM_001367497; NR_160034; NM_153697; XM_047446285; |
| | XM_047446287; XM_047446290; NM_001367495; XM_005246947; NM_001195144; |
| | XM_006712838; XM_047446288; XM_047446289; XR_923062; XM_047446283; |
| | NM_001367496; XM_047446286; XM_024453216; XM_005246948; XM_047446284 |
| FAM78A | NM_001400581; NM_001400583; NM_001400588; XM_011518568; NM_001400584; |
| | NM_001400585; NM_001400593; NM_001400591; XM_047423250; NM_001400589; |
| | NM_001400590; NM_001400592; NM_001400595; NM_033387; NM_001400582; |
| | NM_001400586; NM_001400594; NM_001399459; NM_001400587 |
| FCAR | XM_017026474; NM_133273; NM_133274; XM_011526625; NM_002000; NM_133271; |
| | NM_133278; XM_047438407; NM_133269; XM_047438406; NM_133272; NM_133280; |
| | NM_133277; NM_133279 |
| TNFAIP3 | XM_024446533; XM_047419285; XM_011536095; XM_024446532; XM_047419282; |
| | XM_047419283; NM_006290; XM_011536096; NM_001270507; XM_005267119; |
| | XM_047419284; NM_001270508 |
| HCLS1 | NM_005335; NM_001292041 |
| ARHGAP30 | NM_001025598; NM_001287602; XM_005245070; NM_001287600; NM_181720; |
| | XM_011509391; XM_047417140; XM_005245073 |
| CD3E | NM_000733 |
| MYO1F | XM_011528028; XR_936181; NM_001348355; XM_047438852; XM_011528027; |
| | XR_936182; XR_001753692; NM_012335; XM_011528024 |
| FMNL1 | XM_006722064; XM_047436644; NM_005892; XM_006722062; XM_006722069; |
| | XM_047436641; XM_006722070; XM_047436637; XM_047436642; XM_047436643; |
| | XM_011525179; XM_047436640; XM_006722065; XM_047436639; XM_011525180; |
| | XM_047436638; XM_006722063; XM_047436646; XM_006722066; XM_047436645 |
| ITGAM | XR_950796; NM_000632; XM_011545850; XM_011545851; XM_017023216; |
| | NM_001145808; XM_006721045; XR_007064878 |
| TRAT1 | NM_016388; NM_001317747 |
| SELPLG | NM_003006; NM_001206609 |
| EVI2B | NM_006495 |
| NCKAP1L | NM_001184976; NM_005337 |
| PRKCQ | XM_005252497; NM_001282645; NM_001282644; NM_001323267; XM_005252496; |
| | NM_001323266; NM_006257; NM_001242413; NM_001323265 |
| KLRC4 | NM_013431 |
| CCL3 | NR_168496; NR_168495; NM_002983; NR_168494 |
| P2RY8 | NM_178129; XM_011545632; XM_04744203 1; XM_047442729; XM_005274429; |
| | XM_006724864; XM_006724443; XM_011546179; XM_005274778 |
| KIR2DL4 | NM_001080770; NM_001080772; NM_002255; NM_001258383 |
| DEFA1B | NM_001042500.2; NM_001302265.2 |
| MMP19 | NM_002429.6; NM_001272101.2; NR_073606.2 |
| FCGR1A | NM_001378804; NM_001378805; NM_001378807; NM_001378810; NR_166122; |
| | NR_166123; NM_001378809; NM_001378811; NM_001378808; NR_166121; NM_000566; |
| | NM_001378806 |
| LILRB3 | NM_006864.4; NM_001081450.3; NM_001320960.2; NR_135493.2; NR_135494.2; |
| | NR_135495.2; NR_135496.2 |
| RASSF2 | XM_047440622; NM_170773; XM_017028152; XM_017028153; XM_047440619; |
| | XM_011529411; XM_017028151; XM_017028149; XM_047440618; XM_047440621; |
| | NM_170774; XM_005260895; XM_011529410; XM_017028150; NM_014737; |
| | XM_047440620 |
| ZAP70 | NM_001378594; NM_207519; XR_007081582; NM_001079; XM_047445775; |
| | XM_047445774; XM_047445776; XR_007081583 |
| KLRK1 | NM_007360.4 |
| LTA | NM_000595; NM_001159740; XM_047418773 |
| IL2RA | NM_001308243; NM_001308242; NM_000417 |
| CD83 | NM_001040280; NM_001251901; NM_004233 |
| IKZF1 | XM_011515064; XM_011515071; XM_011515073; XM_017011668; XM_047419729; |
| | XM_047419732; XM_047419733; XM_047419741; NM_001220767; NM_001291841; |
| | NM_001291842; NM_001220775; XM_011515061; XM_011515063; XM_011515065; |
| | XM_011515072; XM_011515078; XM_047419723; XM_047419730; XM_047419736; |
| | XM_047419742; XM_047419749; NM_001291837; NM_001291846; NM_001220774; |
| | XM_011515062; XM_011515066; XM_047419726; XM_047419739; XM_047419740; |
| | XM_047419743; XM_047419746; XM_047419747; NM_006060; NM_001220772; |
| | XM_047419748; NM_001220768; NM_001220771; NM_001291843; NM_001291845; |
| | XM_011515060; XM_011515067; XM_047419731; XM_047419738; NM_001291838; |
| | NM_001291840; NM_001220769; XM_011515058; XM_011515059; XM_011515070; |
| | XM_047419728; XM_047419734; XM_047419735; XM_047419745; NM_001220765; |
| | NM_001220770; NM_001291839; NM_001291844; XM_011515077; XM_047419724; |
| | XM_047419744; XM_047419750; NM_001220773; XM_011515074; XM_047419725; |
| | XM_047419727; NM_001291847; NM_001220766; NM_001220776 |
| GNLY | NM_001302758; XM_005264085; XM_047442947; NM_006433; XM_005264084; |
| | NM_012483 |
| BTG2 | NM_006763 |
| TRAF1 | NM_001190945; NM_005658; NM_001190947 |
| TNFAIP8L2 | NM_024575 |
| HSPA6 | NM_002155 |
| SLAMF1 | XM_047428486; XM_047428490; NR_104400; XM_005245456; XM_017002130; |
| | XM_047428487; NR_104401; NM_003037; NM_001330754; NR_104399; XM_017002131 |
| ADAM8 | XM_047424425; XM_047424426; XM_047424423; NM_001164490; NM_001164489; |
| | XM_047424424; NM_001109; XR_007061938 |
| IL2RB | NM_000878; NM_001346223; NM_001346222 |
| SIGLEC9 | XM_011526732; NM_014441; NM_001198558; XM_047438615; XM_047438616 |
| TREM2 | NM_001271821; NM_018965 |
| ACAP1 | NM_014716; XM_047437152; XM_047437151; XM_047437150 |
| ACP5 | XM_047438944; NM_001111035; NM_001322023; NM_001611; NM_001111034; |
| | NM_001111036; XM_047438945; XM_005259938; XM_011528069 |
| TNFSF8 | NM_001252290; NM_001244 |
| GZMA | NM_006144 |
| ARHGAP9 | XM_011538656; XM_011538659; XM_047429337; XM_047429339; XM_047429340; |
| | NM_001367422; NM_001367424; XM_047429334; NM_001367423; NM_001367425; |
| | NM_001367426; NM_001319851; XM_047429329; XM_047429332; XM_047429333; |
| | NM_001319852; XM_005269083; NM_001080157; NM_001319850; XM_047429330; |
| | XM_047429336; XM_047429335; NM_001080156; XM_047429331; XM_047429338; |
| | NM_032496 |
| MZB1 | XM_047417264; NM_016459 |
| TMEM176A | XM_047420570; XM_011516376; XM_011516378; XM_024446824; NM_018487 |
| ALOX5 | XM_047424936; NM_001320861; NM_001256153; NM_001256154; NM_001320862; |
| | XM_047424937; XM_047424934; NM_000698 |
| CXCR2 | XM_047444190; XM_047444188; NM_001557; NM_001168298; XM_005246530; |
| | XM_047444189; XM_017003991; XM_047444191; XM_047444187 |
| PRF1 | NM_005041; NM_001083116 |
| CDH5 | XM_047433469; XM_047433470; NM_001114117; NM_001795; XM_047433471; |
| | XM_011522801 |
| ICAM2 | NM_001099786; NM_000873; NM_001099789; NM_001099787; NM_001099788 |
| IGHG3 | NG_001019.6 |
| TNIP3 | NM_001244764; XM_017008625; NM_001128843; XM_047416181; XM_047416182; |
| | NM_024873; XM_011532256; XM_011532257 |
| ESAM | NM_138961 |
| LILRB2 | NM_001278403; NM_001278406; NM_005874; NM_001080978; NM_001278404; |
| | NM_001278405; NR_103521 |
| FCER2 | NM_002002; NM_001220500; XM_005272462; NM_001207019 |
| CCL5 | NM_001278736; NM_002985 |
| ICOS | XR_007073112; XM_047444022; NM_012092 |
| IL7R | XM_047417149; XM_005248299; XM_047417150; NR_120485; NM_002185 |
| OSM | NM_001319108; XM_047441387; NM_020530 |
| FYN | NM_001242779; XM_017010651; XM_047418565; XM_047418571; XM_005266892; |
| | XM_047418561; XM_047418562; XM_047418563; XM_047418566; XM_047418569; |
| | XM_047418572; NM_001370529; XM_047418570; NM_153047; NM_153048; |
| | XM_047418567; XM_047418568; XM_047418573; XM_017010650; NM_002037; |
| | XM_017010652; XM_017010653 |
| TNF | NM_000594 |
| SIGLEC10 | XM_005259366; XM_047439604; NM_001171160; XM_005259367; XM_047439600; |
| | NM_001171156; NM_001171159; NM_001171161; XM_047439602; XM_047439605; |
| | NM_001171158; XM_047439601; XM_047439603; NM_001171157; NM_001322105; |
| | NM_033130 |
| SPN | XM_047426248; XM_047426251; NM_001293634; XR_007062437; NM_001367390; |
| | NM_021008; XR_007062436; XM_011519842; XM_047426250; XM_047426249 |
| DEFA1 | NM_004084 |
| CLEC12A | NM_138337.6; NM_201623.4; NM_001207010.2; NM_001300730.2 |
| SAMD3 | NM_001017373.4; NM_001258275.3; NM_001277185.2 |
| RGS2 | NM_002923 |
| TSC22D3 | NM_198057; XM_011530884; XM_005262102; NM_001015881; XM_005262100; |
| | NM_004089; NM_001318470; XM_047441897; NM_001318468; XM_005262103; |
| | XM_047441896; XM_047441898 |
| COL6A3 | NM_057164; NM_057167; NM_057166; NM_004369; NM_057165 |
| MFAP5 | NM_001297709; NR_123733; NR_123734; NM_001297711; NM_003480; NM_001297710; |
| | NM_001297712 |
| MT1G | NM_001301267; NM_005950 |
| GBP1 | NM_002053 |
| TNFSF13B | NM_006573; XM_047430055; NM_001145645 |
| MS4A1 | NM_021950; NM_152866; NM_152867 |
| VSIG4 | NM_007268; NM_001184830; NM_001184831; NM_001100431; NM_001257403 |
| MXD1 | NM_001202513; NM_001202514; NM_002357 |
| PLXNC1 | XM_047428050; XM_011537730; NR_037687; NM_005761; XM_006719186; |
| | XM_011537731 |
| RGS1 | NM_002922 |
| LY9 | XM_011509556; XM_047420762; NM_001261457; XM_047420755; XM_017001303; |
| | XM_047420771; NM_001033667; NM_001261456; XM_047420753; XM_047420764; |
| | XM_017001304; XM_017001299; NM_002348; XM_011509549; XM_011509560; |
| | XM_017001301; XM_047420765 |
| IL13 | NM_001354991; NM_001354992; NM_002188; NM_001354993 |
| CD86 | NM_001206924; NM_006889; NM_176892; NM_001206925; NM_175862 |
| VPREB3 | NM_013378 |
| FOLR2 | NM_001113535; NM_000803; XM_005273856; XM_047426683; NM_001113534; |
| | NM_001113536 |
| CYTH4 | NM_013385; NM_001318024 |
| SPON2 | NM_012445; NM_001199021; NM_001128325 |
| AC233755.1 | XM_011546198.2 |
| CLEC14A | NM_175060 |
| KLRD1 | XM_011520651; XM_047428824; XM_047428821; NM_001351062; XM_047428823; |
| | NR_147039; XM_047428825; NM_001114396; NM_001351060; NM_002262; NR_147038; |
| | NR_147040; XM_024448974; XM_047428822; NM_001351063; NM_007334 |
| CYBB | XM_047441855; NM_000397 |
| CCR8 | NM_005201 |
| HLA-C | NM_002117; NM_001243042 |
| HLA-DMA | NM_006120 |
| HLA-DRA | NM_019111 |
| ITGB7 | NM_000889; XM_005268851; XM_005268852; NR_104181; XM_047428800 |
| LCP1 | XM_047430303; XM_047430305; NM_002298; XM_047430304; XM_005266374 |
| FPR3 | NM_002030; XM_011526687 |
| GIMAP2 | NM_015660 |
| HLA-DQA1 | NM_002122; XM_006715079 |
| EMILIN2 | NM_032048; XM_047437887; XM_047437886; XM_047437884; XM_047437885 |
| N4BP2L1 | NM_001079691; NM_001286460; NM_001353631; XM_047430761; NM_001286461; |
| | NM_001353633; NM_001353629; NM_001353635; NR_148480; XM_047430763; |
| | NM_001353634; NM_001353636; NR_148475; XM_047430762; NM_001286459; |
| | NM_001353630; NR_148477; XM_017020838; NM_001353627; NM_001353632; |
| | NM_001353637; NM_052818; NR_148478; XM_047430764; NM_001353628; |
| | XM_011535303; NR_148476; NR_148479 |
| HLA-DPA1 | NM_001405020; NM_001242525; NM_033554; XM_047418717; NM_001242524 |
| FGD3 | NM_001286993; NM_001369951; NM_001083536; NM_033086; NM_001369952 |
| ADGRG3 | XM_047433782; XM_011522954; XM_047433781; XM_047433783; XM_005255842; |
| | XM_011522953; XM_047433780; XM_006721170; NM_001308360; NM_170776 |
| FAM65B | NM_001286446; XM_006715275; XM_011515012; NM_001346031; XM_017011524; |
| | XM_047419592; XM_006715281; XM_047419590; XM_047419591; XM_047419593; |
| | NM_001286445; NM_001286447; NM_001346032; XM_006715279; NM_014722; |
| | NM_015864 |
| NCF1 | NM_000265 |
| CD2 | NM_001328609; NM_001767 |
| FASLG | NM_001302746; NM_000639 |
| LIMD2 | XM_005257703; XM_006722124; XM_047436853; NM_030576; XM_005257705 |
| CD160 | NM_007053; XM_005272929; XM_011509104; NR_103845 |
| CD209 | NR_026692; NM_001144895; NM_001144894; NM_001144893; NM_021155; |
| | NM_001144896; NM_001144897; NM_001144899 |
| XCL2 | NM_003175 |
| PNRC1 | NM_006813; XM_047418106 |
| CTSS | NM_004079; NM_001199739 |
| ALOX5AP | XM_017020522; NM_001204406; NM_001629 |
| WIPF1 | NM_001077269; NM_001375832; XM_047445752; XM_047445755; NM_001375839; |
| | NM_003387; XM_047445750; XM_047445751; XM_047445757; NM_001375833; |
| | NM_001375837; XM_047445749; XM_047445753; XM_047445754; NM_001375836; |
| | NM_001375838; XM_047445756; NM_001375834; NM_001375835 |
| POU2F2 | XM_047438954; XM_047438963; XM_047438967; XM_047438961; NM_001393935; |
| | XM_017026891; XM_047438955; XM_017026894; NM_001207026; NM_001393934; |
| | NM_001394376; NM_001394378; XM_047438958; XM_047438960; NM_001247994; |
| | XM_011527041; XM_047438953; XM_047438959; XM_047438962; XM_047438965; |
| | NM_001207025; XM_017026892; XM_011527042; XM_047438957; XM_047438966; |
| | NM_001393936; NM_002698; XM_047438956; XM_047438964; XM_047438968; |
| | NM_001394377 |
| ROBO4 | NM_019055; XM_006718861; NM_001301088; XM_011542875 |
| EOMES | XM_005265510; NM_001278182; NM_005442; NM_001278183 |
| ORM1 | NM_000607 |
| SIGLEC5 | NM_001384708; NM_001384709; NM_003830; XM_047446914; XM_047446915 |
| ITGAX | NM_001286375; XM_024450263; XM_011545852; XM_011545854; XM_047434075; |
| | NM_000887; XM_047434074 |
| ORM2 | NM_000608 |
| CXCL8 | NM_000584; NM_001354840 |
| CX3CR1 | NM_001171174; NM_001337; XM_047447538; NM_001171171; NM_001171172 |
| ZBP1 | NM_001323966; XR_007067479; NM_001160417; NM_001160419; XM_011529058; |
| | NM_030776; NR_136660; XR_007067477; XR_007067480; XR_00706748 1; |
| | XM_047440526; XM_047440525; XM_047440527; NM_001160418; XR_001754408; |
| | XR_007067478 |
| GPR18 | NM_001098200; NM_005292 |
| APLN | NM_017413 |
| CD226 | NM_006566; XM_047437274; NM_001303619; XM_047437275; XM_047437276; |
| | XM_006722374; XM_005266642; XM_047437277; NM_001303618 |
| IL2RG | XM_047442089; NM_000206 |
| CTSK | NM_000396 |
| LCK | XM_047420403; XM_011541453; XM_024447046; XM_047420399; NM_001330468; |
| | XM_024447047; NM_005356; NM_001042771 |
| GZMH | NM_001270781; NM_001270780; NM_033423 |
| C1orf162 | NM_001300835; NM_001300834; XM_047446258; NM_174896 |
| APOBR | NM_018690 |
| PEEK | NM_002664; XM_047444772 |
| TIGIT | XM_047447672; XM_047447671; NM_173799 |
| NLRC3 | XM_047433771; NM_178844; XM_047433769; NR_075083; XM_047433770 |
| SMAP2 | NM_001198978; NM_001198979; XM_047428013; XM_011541960; XM_047428009; |
| | XM_047428012; XM_047428015; XM_047428010; XM_047428011; NM_001198980; |
| | XM_047428016; XM_047428017; XM_047428014; NM_022733 |
| GZMM | NM_001258351; NM_005317 |
| LSP1 | NM_001242932; NM_001013255; NM_001289005; NM_001013254; NM_002339; |
| | NM_001013253 |
| HLA-DMB | NM_002118 |
| IGHG1 | NG_001019.6 |
| AMICA1 | NR_104479; NM_001098526; NM_153206; NM_001286570; NM_001286571 |
| NKG7 | XM_006723228; XM_005258955; NM_001363693; NM_005601 |
| TMIGD2 | XM_047438167; NR_172632; NM_001395549; NM_001308232; NR_172630; |
| | NM_001169126; NM_144615; NR_172631 |
| IL9 | NM_000590 |
| SLCO2B1 | NM_007256; XM_017017157; NM_001145211; NM_001145212; XM_047426333; |
| | XM_047426334 |
| CD79B | NM_001039933; NM_021602; NM_000626; NM_001329050 |
| WAS | XM_011543977; XM_047442434; XM_047442432; XM_017029786; XM_047442433; |
| | NM_000377 |
| STAB1 | XM_047447774; XM_006713065; XM_005264974; XM_047447777; NM_015136; |
| | XM_005264973; XM_047447775; XM_047447776 |
| LAT2 | XM_047420801; NM_014146; XM_011516558; NM_032464; NM_032463 |
| SRGN | NM_001321054; NM_001321053; NM_002727 |
| FAM129C | XM_011527789; XM_011527781; XM_017026454; NM_001321828; XM_017026453; |
| | XM_011527786; XM_047438389; NM_001098524; XM_047438388; NM_173544; |
| | XM_017026457; XM_047438390; NM_001321826; XM_011527787; NM_001321827; |
| | NM_001363609 |
| BIN2 | XM_047428968; XR_001748746; NM_001364780; NM_001290008; NM_001290007; |
| | NM_001290009; NM_001364779; NM_001364781; NM_016293 |
| SELE | NM_000450 |
| LILRA5 | NM_181985; NM_021250; NM_181879; NM_181986 |
| CCR3 | NM_001164680; NM_001837; NM_178328; NM_178329; XM_017005685; XM_006712960 |
| CCL3L3 | NM_001001437.4 |
| TBX21 | NM_013351 |
| CARD16 | NM_001394580; NM_052889; NM_001017534 |
| LRRC25 | XM_005259739; NM_145256 |
| KIR2DL3 | NM_015868; NM_014511 |
| IFI30 | NM_006332 |
| HLA-DRB4 | NM_021983 |
| LCP2 | NM_005565; XM_047417171 |
| STX11 | XM_047419437; XM_011536213; XM_011536217; XM_047419436; XM_011536214; |
| | XM_047419438; NM_003764; XM_047419440; XM_011536218; XM_047419439; |
| | XM_047419441 |
| GBP2 | NM_004120 |
| VNN3 | NM_001291703; NM_001368152; NM_001368154; NR_173393; NR_173395; NM_018399; |
| | NR_173392; NM_001368156; NM_001291702; NR_173396; NM_001368151; |
| | NM_001368155; NM_001368149; NM_001368150; NR_173391; NM_078625; NR_173394 |
| GLIPR2 | XM_047422807; NR_104637; NR_104641; NM_001287013; NM_001287010; |
| | NM_001287014; NM_022343; NR_104640; XM_024447416; NM_001287011; NR_104638; |
| | NM_001287012; NR_104639 |
| TRGC1 | NG_001336.2 |
| IKZF3 | NM_001257411; NM_001284516; NM_001257414; NM_001284515; NM_183230; |
| | NM_183231; NM_183232; NM_001257412; NM_001257413; NM_001257408; |
| | NM_001257409; NM_001284514; NM_183228; XM_047435625; NM_001257410; |
| | NM_012481; NM_183229 |
| MS4A4A | NM_001243266; NM_148975; NM_024021 |
| GREM1 | NM_001368719; NM_013372; NM_001191323; NM_001191322 |
| HP | NM_001126102; NM_005143; NM_001318138 |
| POU2AF1 | XM_006718860; XM_017017932; XM_006718859; XM_005271593; XM_047427137; |
| | NM_006235 |
| ATG16L2 | XM_006718733; XM_011545332; XM_047427840; XM_005274376; NM_001318766; |
| | NM_033388; XM_011545333; XM_047427842; XM_011545334; XM_047427841; |
| | XM_006718732 |
| CD40LG | NM_000074 |
| IGSF6 | NM_005849 |
| SPIB | NM_001243999; NM_001243998; NM_001244000; NM_003121 |
| STAT5A | NM_001288719; XM_047436591; XM_047436590; NM_001288720; NM_003152; |
| | XM_047436589; NM_001288718; XM_047436588; XM_005257624 |
| PTPRC | XM_047426420; NM_001267798; NM_002838; NR_052021; NM_080922; |
| | XM_006711473; XM_006711474; XM_047426417; XM_047426409; XM_047426381; |
| | XM_006711472; XM_047426398; XM_047426415; NM_080921 |
| SLA | NM_006748; XM_047422110; NM_001045556; XM_047422108; NM_001045557; |
| | XM_047422109; NM_001282964; XM_047422107; NM_001282965 |
| CD4 | NM_001195014; NM_001382707; NM_001382714; NM_001195016; NM_000616; |
| | NR_036545; NM_001195015; NM_001382705; NM_001195017; NM_001382706 |
| DENND1C | XM_047439458; XM_047439459; XM_047439460; XM_024451727; NM_001290331; |
| | XM_006722906; XM_011528318; XM_006722905; NM_024898 |
| RNASE6 | XM_017021566; NM_005615 |
| TMC8 | XM_024450618; XM_024450620; XM_047435479; XM_047435494; XM_047435488; |
| | XR_007065273; XM_024450623; XM_047435492; XM_017024244; XM_024450619; |
| | XM_024450624; XM_047435482; XM_047435489; XR_002957973; XR_007065271; |
| | XM_024450622; XM_047435484; XM_047435485; XM_047435487; XM_047435491; |
| | XM_047435493; XR_007065274; XM_024450621; XR_007065276; XM_024450617; |
| | XM_024450626; XM_024450627; XM_047435478; XM_047435480; XM_047435481; |
| | XM_047435486; XM_047435490; XR_007065272; XM_024450625; NM_152468; |
| | XR_007065275 |
| PGLYRP1 | NM_005091 |
| LAIR1 | NM_001289025; NM_002287; XM_017026803; XM_047438810; NM_001289023; |
| | NM_001289026; NM_001289027; NM_021706; XM_047438811; NR_110280; |
| | XM_047438812; NM_021708; NR_110279 |
| ZNF683 | XM_011541198; XM_005245830; XM_017000956; NM_001114759; NM_173574; |
| | XM_005245832; XM_047417136; XM_005245828; XM_006710555; XM_017000954; |
| | XM_017000957; NM_001307925 |
| CD53 | NM_000560; NM_001040033; XM_047435014; XM_047435015; NM_001320638; |
| | XM_047435013 |
| IGKC | NG_000834.1 |
| KLRC1 | NM_002259; NM_007328; NM_001304448; NM_213657; NM_213658 |
| MMP1 | NM_001145938; NM_002421 |
| CXCR1 | NM_000634 |
| GIMAP4 | NM_018326; NM_001363532 |
| IL10RA | XM_047426883; NM_001558; XM_047426884; XM_047426882; NR_026691 |
| FGFBP2 | NM_031950 |
| TRBC2 | NG_001333.2 |
| PDGFRA | XM_047415767; NM_001347828; NM_001347829; XM_005265743; XM_017008281; |
| | NM_001347827; XM_047415766; NM_001347830; NM_006206; XM_006714041 |

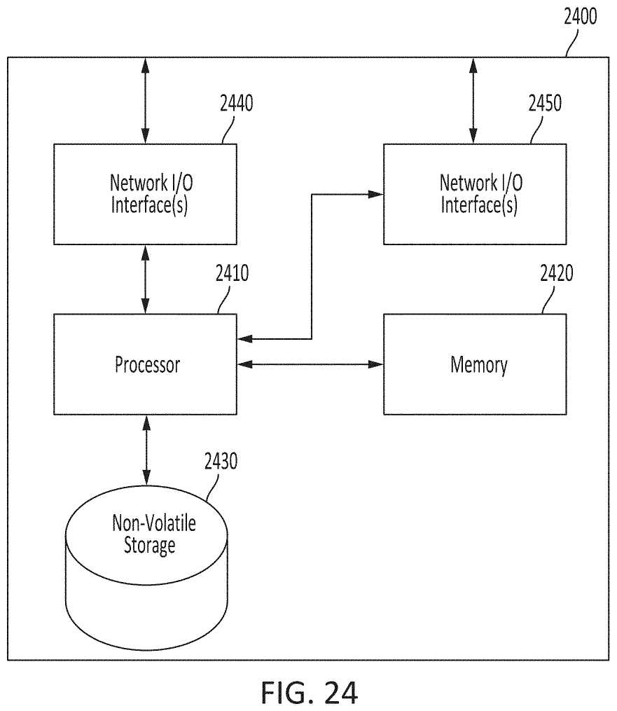

FIGS. 2A-2C are flowcharts depicting illustrative processes (e.g., process 200, 220, and 250) for estimating tumor expression levels of genes in tumor cells in a biological sample, according to some embodiments of the technology described herein. The processes may be performed by any suitable computing device(s). For example, the processes may be performed by a laptop computer, a desktop computer, one or more servers, in a cloud computing environment, computing device 2400 as described herein within respect to FIG. 24, or in any other suitable way.

FIG. 2A is a flowchart depicting a process 200 for estimating tumor expression levels of genes in tumor cells in a biological sample using machine learning, according to some embodiments of the technology described herein.

In the embodiment of FIG. 2A, process 200 begins at act 202, where expression data for a set of genes is obtained. The expression data may be of any suitable type and, for example, may include any type of expression data described herein including at least with respect to FIG. 1 and the section “Expression Data”. For example, the expression data may include a total expression level for a gene in the set of genes. The total expression level for a gene may reflect the combined expression of the gene in both tumor and TME cells of the biological sample. As such, the total expression level for a particular gene does not distinguish between the expression of that particular gene in tumor cells and the expression of that particular gene in TME cells.

In some embodiments, the set of genes includes genes associated with tumor cells, and the expression data includes total expression levels for the genes associated with tumor cells. In some embodiments, the set of genes includes at least 10, at least 25, at least 30, at least 40, at least 50, at least 60, at least 75, at least 100, at least 150, or more genes associated with tumor cell. For example, the set of genes may include a subset (e.g., at least some or all) of the genes listed in the Table 1, and the expression data may include total expression levels for those genes.

In some embodiments, the set of genes also includes genes associated with TME cells, and the expression data includes total expression levels for the genes associated with TME cells. In some embodiments, the set of genes includes at least 10, at least 25, at least 30, at least 40, at least 50, at least 60, at least 75, at least 100, at least 150, at least 200, at least 250, at least 300, at least 350, at least 400, or more genes associated with TME cells. For example, the set of genes may include a subset (e.g., at least some or all) of the genes listed in the Table 2, and the expression data may include total expression levels for those genes.

In some embodiments, the expression data is obtained using any suitable techniques from any suitable location such as, for example, a data store (e.g., expression data store 446 of FIG. 4). For example, the expression data may have been previously-obtained in a remote setting and uploaded to the data store. Additionally or alternatively, the expression data may be obtained directly from a sequencing platform (e.g., sequencing platform 444 of FIG. 4) used to obtain the expression data.

Process 200 then proceeds to act 204, where tumor expression levels of genes associated with tumor cells are determined. In some embodiments, determining a tumor expression level for the genes includes using machine learning models corresponding, respectively, to the genes associated with tumor cells. For example, determining a first tumor expression level for a first gene includes using a first machine learning model corresponding to the first gene.

In some embodiments, act 204 includes determining a tumor expression level for a set (e.g., at least some or all) of the genes listed in Table 1. For example, act 204 may include determining a tumor expression level for at least 10, at least 25, at least 30, at least 40, at least 50, at least 60, at least 75, at least 100, at least 150 or all of the genes listed in Table 1. Techniques for determining a tumor expression level for a gene are described herein, including at least with respect to FIGS. 2B-2C.

At act 206, the tumor expression levels of the genes associated with tumor cells are output. In some embodiments, the tumor expression levels are made accessible to a user (e.g., a clinician, a researcher, etc.). For example, the tumor expression levels may be displayed via a user interface (e.g., a graphical user interface (GUI)), stored locally in non-transitory storage medium, stored in a remote database or a cloud storage environment, and/or transmitted to one or more external computing devices.

In some embodiments, the tumor expression level of a particular gene is associated with one or more anti-cancer therapies. For example, a particular therapy may be known to effectively treat tumors expressing the particular gene. Additionally or alternatively, a particular therapy be known to ineffectively treat tumors expressing the particular gene.

Accordingly, in some embodiments, at act 208 the output tumor expression levels are used to identify an anti-cancer therapy for administration to the subject. In some embodiments, this includes determining whether an output tumor expression level satisfies one or more criteria. In some embodiments, the criteria vary for each gene and its associated therapies. For example, a therapy may effectively treat tumors that express a particular gene (e.g., a tumor expression level of the gene that exceeds 0). By contrast, a therapy may effectively treat tumors that overexpress or under-express a gene (e.g., tumor expression levels that exceed or fall below an average expression of the gene).

Aspects of the disclosure relate to identification and/or selection of therapeutic agents (e.g., anti-cancer therapies) that are associated with a particular gene. A therapeutic agent that is “associated with a particular gene” refers to a therapeutic agent that interacts (e.g., binds to, inhibits activity or function, decreases activity or function, or alters activity or function) with a gene product (e.g., a nucleic acid such as DNA or RNA, a peptide, protein, etc.) expressed by the particular gene. For example, a therapeutic agent associated with a gene encoding a kinase (e.g., ALK) may bind to or interact with a nucleic acid (e.g., mRNA transcribed from the gene (e.g., ALK gene) or a protein (e.g., ALK protein) expressed by the gene. In some embodiments, a therapeutic agent associated with a particular gene may interact directly (e.g., bind to or directly inhibit) the particular gene. In some embodiments, a therapeutic agent associated with a particular gene may interact indirectly with the particular gene (e.g., bind to or inhibit a modulator of the particular gene). A therapeutic agent may be a small molecule (e.g., small molecule inhibitor, for example a kinase inhibitor, DNA methyltransferase inhibitor, topoisomerase inhibitor, etc.), nucleic acid (e.g., inhibitory nucleic acid such as dsRNA, siRNA, miRNA, etc., or a therapeutic mRNA), peptide, or protein (e.g., antibody, toxin, etc.). In some embodiments, the therapeutic agent is approved by a government regulatory agency (e.g., the US Food and Drug Administration) for treatment of cancer. FDA-approved agents are known in the art and are described, for example in the FDA Orange Book or FDA Purple Book. Table 3 lists therapies associated with tumor expression of particular genes. In some embodiments, act 208 comprises identifying one or more therapies listed in Table 3.

In some embodiments, implementing process 200 may include additional or alternative steps that are not shown in FIG. 2A. For example, executing process 200 may include every act included in the example flowchart. Alternatively, process 200 may include only a subset of the acts included in the example flowchart (e.g., acts 202 and 206, acts 202, 204, 206, and 208, acts 202, 204 and 206, etc.).

TABLE 3
Therapies and cancers associated with tumor expression of
particular genes.

| Gene | Cancer Types | Therapy |
|---|---|---|
| ALK | anaplastic large-cell lymphoma, | Crizotinib |
| | inflammatory myofibroblastic tumors, | |
| | diffuse large B-cell lymphoma, | |
| | non-small-cell lung cancer (NSCLC), | |
| | colorectal, breast carcinomas | |
| PTK7 | atypical teratoid rhabdoid tumors, | PTK7 Antibody-drug |
| | breast cancer, cholangiocarcinoma, | conjugate, PF-06647020 |
| | colorectal cancer, esophageal | |
| | squamous cell carcinoma and gastric | |
| | cancer, cholangiocarcinoma | |
| PIK3CG | colorectal cancers, | Combination of |
| | colon cancers, | paclitaxel (PTX) and |
| | claudin-low breast cancer | AS-605240 |
| CDH1 | hereditary diffuse gastric cancer, | Suppressor-tRNA |
| | lobular breast cancer | |
| MKI67 | bladder cancer, CNS and brain, breast | Ki-67 labeling index for |
| | cancer (BC), colorectal cancer (CRC), | diagnosis and prognosis |
| | cervical cancer, esophageal cancer | assessment of cancer |
| | (EC), head and neck cancer (HNC), | patients |
| | gastric cancer (GC), liver cancer, | |
| | ovarian cancer, lung cancer (LC), | |
| | lymphoma, sarcoma, and pancreatic | |
| | cancer compared with noncarcinoma | |
| | tissues. | |
| CCND2 | triple-negative breast cancer and lung | Antroquinonol D |
| | adenocarcinoma, non-small-cell lung | |
| | carcinoma and breast cancer patients | |
| BCL2L2 | Neoplasm | Inferior response to |
| | | navitoclax in cancer. |
| CDK2 | glioblastoma, prostate cancer, B cell | CDK2 inhibition (using |
| | lymphoma, triple-negative breast | CYC065) combined with |
| | cancer | eribulin. |
| PDGFA | liver cancer, breast cancer, and oral | PDGF receptor kinase |
| | squamous cell carcinoma, | inhibitors imatinib or |
| | neuroblastomas, osteosarcoma, and | sunitinib |
| | gastric carcinoma, papillary thyroid | |
| | cancer, cholangiocarcinoma | |
| IGF2 | colorectal, breast, prostate and lung | MABs that bind IGF2 |
| | cancers, hepatoblastoma | |
| FGFR | squamous cell carcinomas of the lung | Prognostic biomarker, |
| | and the head and neck, glioblastoma, | that correlates with |
| | melanoma, breast, prostate, bladder, | parameters of worse |
| | and ovarian cancer | outcome |
| FLNA | malignant mesothelioma, breast | Therapy or others to |
| | cancer | induce cleavage of |
| | | FLNA |
| TOP1 | colon cancer, breast cancer, ovarian | Top1 targeting drugs, |
| | cancer, and recurrent small-cell lung | Enhancement of |
| | cancer | radiotherapy with TOP1 |
| | | drugs (Camptothecin). |
| KMT2E | large intestine, ovary, central nervous | Prognostic marker for |
| | system, and stomach, but | patients with AML |
| | downregulation in others, e.g., the | treated in the AMLSHG |
| | pancreas, thyroid, and breast cancer | 0199 and AMLSHG |
| | | 0295 trials |
| B2M | breast cancer, prostate cancer, lung | Inhibitors targeting the |
| | cancer, renal cancer, multiple | B2M in combination |
| | myeloma, and especially non- | with other immune |
| | Hodgkin’s lymphoma, colorectal | checkpoint molecules. |
| | cancer | |
| ERBB3 | ovarian, breast, prostate, gastric, | Activation of HER3 |
| | bladder, lung, melanoma, colorectal | signaling is one major |
| | and squamous cell carcinoma, | cause of treatment failure |
| | pancreatic carcinoma | to EGFR or anti- |
| | | estrogenbased therapies. |
| MDM2 | bladder carcinoma, non-Hodgkin's | Diagnostic tool or as a |
| | lymphoma, prostate carcinoma, | marker, particularly for |
| | testicular germ cell tumors, soft tissue | tumor stage or grade. |
| | sarcomas | |
| MCL1 | multiple myeloma, leukemia, non- | Gapil et al. extracted 26 |
| | Hodgkin lymphoma, lung cancer | carboxamides from |
| | | natural fislatifolic acid, |
| | | one of which exhibited |
| | | submicromolar affinity |
| | | for MCL-1 and BCL-2, |
| | | and showed moderate |
| | | cytotoxicity in lung |
| | | and breast cancer cell |
| | | lines |
| MYB | myeloid leukemia (AML), non- | Block gene function |
| | Hodgkin lymphoma, colorectal | with antisense oligo- |
| | cancer, and breast cancer, colon | nucleotides |
| | cancer | |
| AURKA | adrenocortical carcinoma (ACC), | Aurora kinase inhibitors |
| | LGG, KICH, kidney renal clear cell | (e.g., AKI-001, |
| | carcinoma (KIRC), kidney renal | BPR1K871, MLN8054). |
| | papillary cell carcinoma (KIRP), liver | Use in clinical drugs and |
| | hepatocellular carcinoma (LIHC), | in combination with |
| | lung adenocarcinoma (LUAD), | radiotherapy. |
| | mesothelioma (MESO), PAAD, | PHA680632 treatment |
| | SARC and uveal melanoma (UVM). | prior to radiation |
| | | treatment leads to an |
| | | additive effect in cancer |
| | | cells, especially in p53- |
| | | deficient cells in vitro or |
| | | in vivo. |
| PTEN | prostate cancer, breast cancer, | PTEN loss has |
| | glioblastoma, malignant melanoma, | previously been reported |
| | endometrial, prostate, breast, | to be prognostic for |
| | colorectal and pancreatic cancer | outcome following |
| | | radiotherapy in prostate |
| | | cancer. PTEN expression |
| | | also a predictive marker |
| | | for targeted therapeutic |
| | | agents including anti- |
| | | EGFR mAbs, |
| | | trastuzumab-based |
| | | chemotherapy in breast |
| | | cancer. |
| STMN1 | breast cancer, lung cancer, ovarian | A variety of target- |
| | cancer, prostate cancer, sarcoma, and | specific anti-stathmin |
| | gastric cancer | effectors, including |
| | | ribozymes and si-RNA |
| | | have been used to silence |
| | | stathmin in vitro as |
| | | singlets and in |
| | | combination with |
| | | chemotherapeutic agents |
| | | where additive |
| | | synergistic interactions |
| | | have been demonstrated |
| | | (e.g., taxanes) |

FIG. 2B is a flowchart depicting a process 220 for determining a tumor expression level of a gene in the tumor cells of the biological sample, according to some embodiments of the technology described herein. In some embodiments, act 204 of process 200 may be implemented using process 220.

Process 220 begins at act 222, where a first set of features for a first gene associated with tumor cells is generated. In some embodiments, generating the first set of features includes including, in the first set of features, at least some of the expression data obtained at act 202 of process 200. The included expression data may include, for example, total expression levels for at least some genes associated with tumor cells. Additionally or alternatively, the included expression data may include total expression levels for at least some genes associated with TME cells. Example techniques for including expression data in the first set of features are described herein including at least with respect to acts 252 and 254 of process 250, depicted in FIG. 2C.

In some embodiments, generating the first set of features for the first gene further includes determining an initial expression level estimate for the first gene in the tumor cells. For example, the initial expression level estimate of the first gene in the tumor cells may represent an estimate of the tumor expression level of the first gene in the tumor cells, prior to using a machine learning model to determine an updated tumor expression level of the first gene. In some embodiments, determining an initial expression level estimate for the first gene includes estimating the TME expression level of the first gene and subtracting the TME expression level estimate of the first gene from the total expression level of the first gene. Example techniques for determining an initial expression level estimate are described herein including at least with respect to act 256 of process 250, depicted in FIG. 2C.

In some embodiments, generating the first set of features for the first gene includes, obtaining a first plurality of RNA percentages for a respective plurality of cell types in the biological sample and including the first plurality of RNA percentages in the first set of features. As referred to herein, in some embodiments, an “RNA percentage” for a particular cell type is indicative of the percent of RNA sequence reads (e.g., obtained using a sequencing platform) that have aligned to a particular gene (e.g., the first gene) that originate from a particular cell type. For example, for the first gene, the RNA percentage for a first cell type is indicative of the percentage of RNA sequence reads that have aligned to the first gene and that originate from cells of the first cell type in the biological sample.

In some embodiments, obtaining the first plurality of RNA percentages for a respective plurality of cell types includes obtaining an RNA percentage for each of a plurality of TME cell types (e.g., neutrophils, fibroblasts, NK cells, etc.) in the biological sample. In some embodiments, obtaining the first plurality of RNA percentages includes obtaining an RNA percentage for tumor cells in the biological sample.

In some embodiments, RNA percentages are obtained using machine learning techniques. Example techniques for determining RNA percentages are described in the section “Cellular Deconvolution”. Some aspects of determining RNA percentages are also described in U.S. Patent Publication No. 2021-0287759, entitled “SYSTEMS AND METHODS FOR DECONVOLUTION OF EXPRESSION DATA”, the entire contents of which is herein incorporated by reference in its entirety.

At act 224, the first set of features is provided as input to a first machine learning model to obtain an output indicative of a TME expression level estimate for the first gene. In some embodiments, the TME expression level estimate is an estimated expression level of the first gene in the TME cells of the biological sample.

In some embodiments, the first machine learning model is of any suitable type. For example, in some embodiments, the first machine learning model may be a gradient boosted machine learning model. The gradient boosted machine learning model may be a gradient boosted decision tree model or using any other suitable type of model as “weak learner” boosted via gradient boosting or any other suitable boosting approach. In some embodiments, the gradient boosted ML model may be trained using a gradient boosting framework such as XGBoost, LightGBM, Catboost, or Adaboost.

It should be appreciated that the first machine learning model need not be a gradient boosted machine learning model and that other types of ML models may be used. For example, in some embodiments, a non-linear regression model (e.g., a logistic regression model), a neural network model, a support vector machine, a Gaussian mixture model, a random forest model, a decision tree model, or any other suitable type of machine learning model, as aspects of the technology described herein are not limited in this respect.

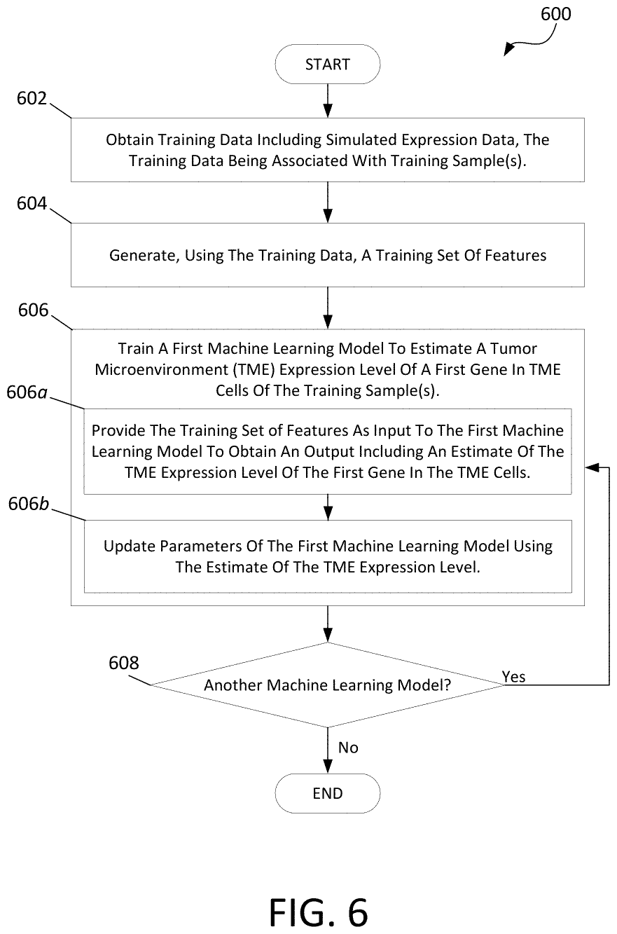

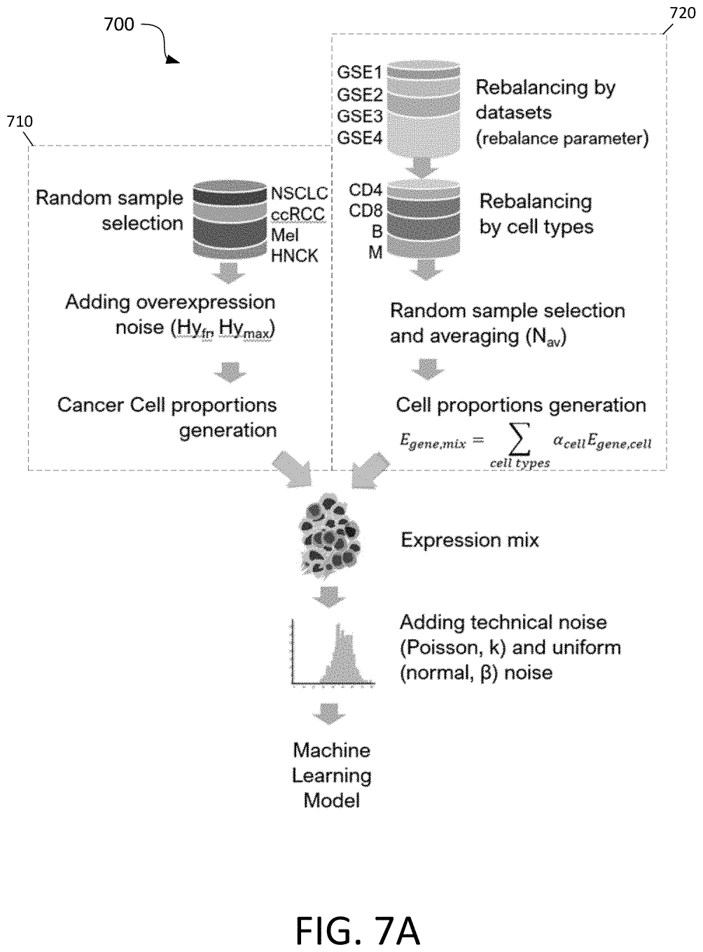

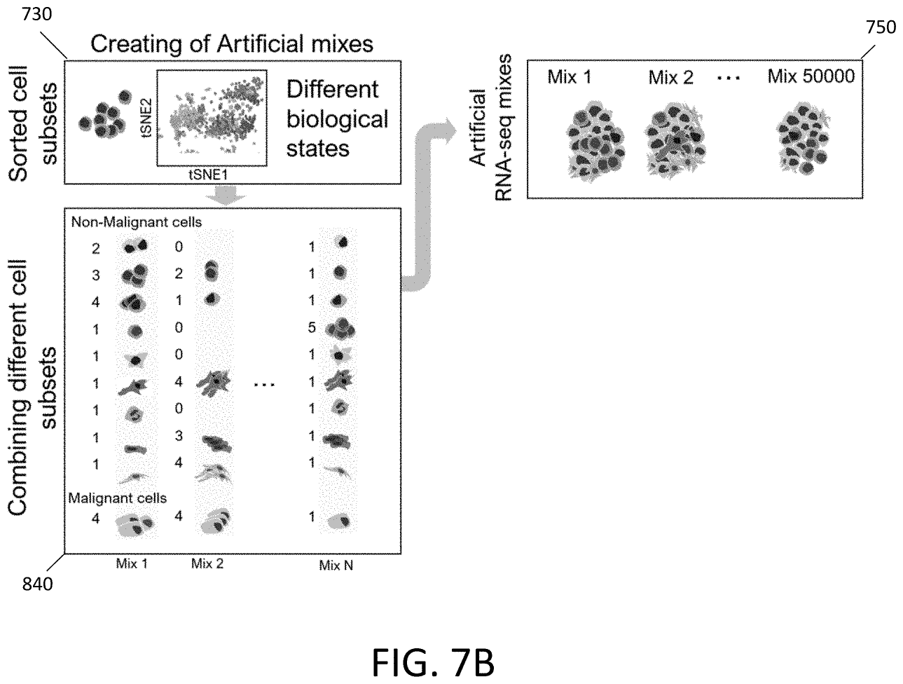

In some embodiments, the machine learning model includes multiple parameters whose values may be estimated using training data. The process of estimating parameter values of parameters in an ML model using training data is referred to as “training” the ML model. In some embodiments, a machine learning model includes one or more hyperparameters in addition to the multiple parameters. Values of the hyperparameters may be estimated during training as well. Example techniques for training the first machine learning model are described herein including at least with respect to FIG. 6 and FIGS. 7A-7B.

At act 226, a first tumor expression level is determined for the first gene. In some embodiments, the first tumor expression level is the predicted expression level of the first gene in tumor cells of the biological sample.

In some embodiments, determining the first tumor expression level includes using the output of the first machine learning model and the total expression level of the first gene (e.g., obtained at act 202 of process 200). This may include, for example, subtracting the TME expression level estimate (TME₁) for the first gene from the total expression level (Total₁) of the first gene to obtain the (unscaled) first tumor expression level (Tumor_{unscaled,1}), as shown in Equation 1.

Tumor_{unscaled,1}=Total₁−TME₁ (Equation 1)

In some embodiments, determining the tumor expression level for the first gene is further based on a predicted RNA percentage of the tumor cells in the biological sample. For example, the RNA percentage (RP₁) of the tumor cells may be used to scale (e.g., divide) the difference between the total expression level and the TME expression level estimate to obtain the (scaled) first tumor expression level, as shown in Equation 2.

$${Tumor}_{scaled , 1} = \frac{{Tumor}_{unscaled , 1}}{{RP}_{1}} \qquad ( Equation 2 )$$

At act 228, process 220 includes determining whether there is another gene associated with tumor cells for which a tumor expression level should be determined. When it is determined, at act 228, that there is another gene for which the tumor expression level is to be determined, acts 222-226 are repeated for the next gene. For example, for a second gene, this would include determining a second set of features, providing the second set of features as input to a second machine learning model to obtain an output indicative of a TME expression level estimate of the second gene in the TME cells, and determining a second tumor expression level for second gene.

FIG. 2C is a flowchart depicting a process 250 for generating a first set of features for the first gene, according to some embodiments of the technology described herein. In some embodiments, act 204 of process 200 may be implemented using process 250. In some embodiments, act 222 of process 220 may be implemented using process 250.

Process 250 begins at act 252, where an initial expression level estimate of the first gene in the tumor cells of the biological sample is obtained.

In some embodiments, the initial expression level estimate is obtained using the expression data obtained at act 202 of process 200. For example, the expression data may be used to obtain, for the first gene, RNA percentages for different TME cell populations (e.g., TME cells of a first type, TME cells of a second type, etc.) in the biological sample. Example techniques for determining RNA percentages are described herein including in the section “Cellular Deconvolution” and in U.S. Patent Publication No. 2021-0287759, entitled “SYSTEMS AND METHODS FOR DECONVOLUTION OF EXPRESSION DATA”, the entire contents of which is herein incorporated by reference in its entirety.

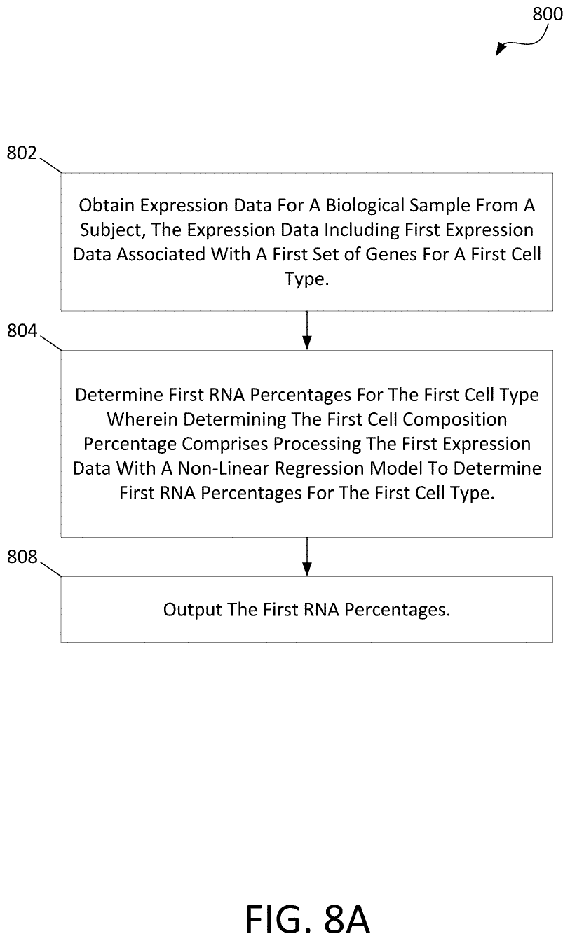

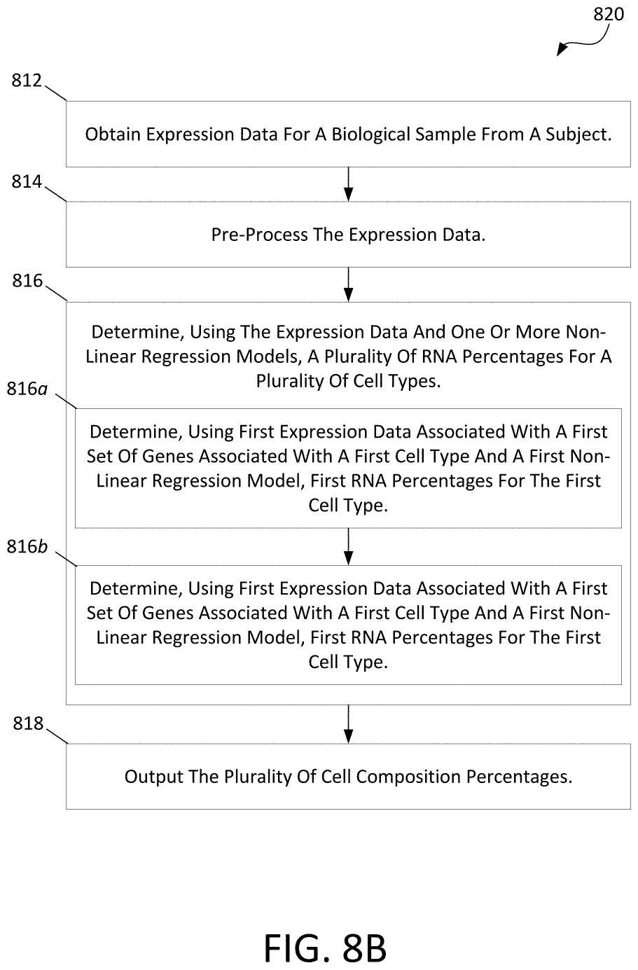

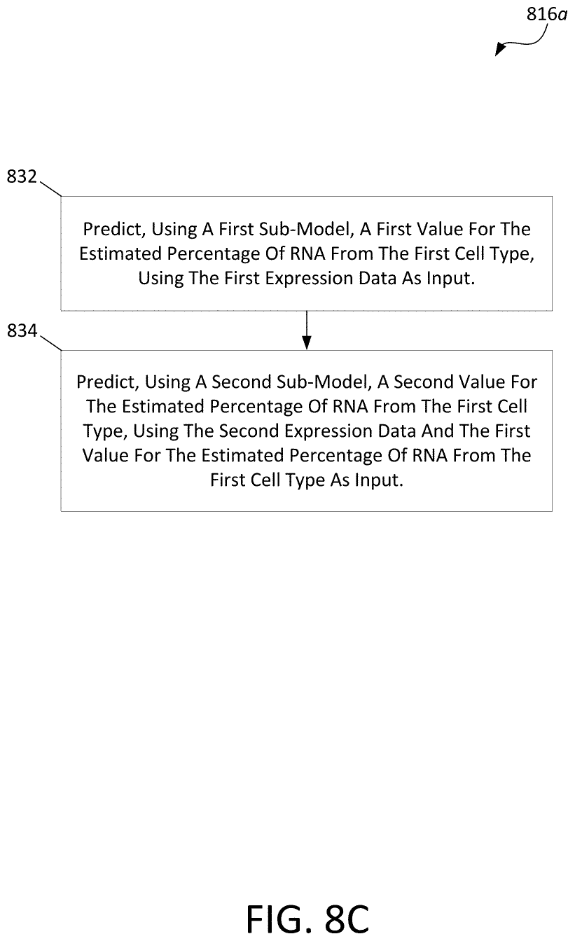

In some embodiments, the initial expression level estimate is further obtained using average expression levels of first gene in each of various TME cell populations (e.g., the average expression levels of the first gene in TME cells of the first type, the average expression levels of the first gene in TME cells of the second type, the average expression levels of the first gene in TME cells of the N^{th} type, etc.) In some embodiments, the average expression level of a gene in a particular cell population is obtained by averaging the expression level of the gene in the cell population across different biological or artificial samples. For example, the average expression level of a gene in a TME cell population may be determined by computing the average expression level of the gene in the TME cell population in the training samples described with respect to FIGS. 7A-7B and FIG. 8. In some embodiments, the average expression level of a gene in a particular cell population has been previously-determined and is stored in a suitable storage medium, such as a database, for example. Therefore, in some embodiments, the average expression levels are obtained from the suitable storage medium. Example average expression profiles for various genes associated with tumor cells are listed in Table 4.

In some embodiments, the RNA percentages and average expression levels are used to determine a weighted sum that represents an initial expression level estimate of the first gene in TME cells of the biological sample. Equation 3 shows an example equation for determining an initial TME expression level estimate (TME_{initial,1}) for the first gene in TME cells of a biological sample including k TME cell populations.

TME_{intiail,1}=Σₖ(RPₖ)*(Expₖ) (Equation 3)

Where RPₖ represents the RNA percentage for the k^{th} TME cell population and EXP_{N} represents the average TME expression level of the first gene in the k^{th} TME cell population.

In some embodiments, the initial TME expression level estimate of the first gene is used to determine the initial tumor expression level estimate of the first gene in the tumor cells of the biological sample. For example, the initial TME expression level estimate of the first gene may be subtracted from the total expression level (Total₁) of the first gene in the biological sample, obtained at act 202 of process 200. Equation 4 shows an example equation for determining an initial expression level estimate (Tumor_{initial,1}) of the first gene in tumor cells the biological sample.

Tumor_{initial,1}=Total₁−TME_{initial,1} (Equation 4)

In some embodiments, the obtained initial expression level estimate of the first gene in the tumor cells is included in the first set of features at act 252 of process 250. For example, the initial expression level estimate may be provided as input to the first machine learning model at act 224 of process 220, along with other features included in the first set of features.

At act 254 of process 250, at least some of the total expression levels for genes associated with tumor cells are included in the first set of features. For example, the total expression levels include those obtained at act 202 of process 200.

In some embodiments, all the obtained total expression levels for the genes associated with tumor cells is included in the first set of features. In some embodiments, only a subset of the total expression levels is included in the first set of features. For example, in some embodiments, total expression levels for at least 10, at least 25, at least 30, at least 40, at least 50, at least 60, at least 75, at least 100, at least 150 or all of the genes listed in Table 1 are included in the first set of features.

In some embodiments, the subset that is included in the first set of features depends on the type of cancer that the subject has or is suspected of having. For example, Table 3 lists genes associated with different types of cancer. For a patient having or suspected of having a particular type of cancer, total expression levels for genes associated with tumor cells and associated with the type of cancer may be included in the first set of features.

In some embodiments, the subset of features to be included in the first set of features is identified as part of training the first machine learning model. Kursa et al. (Boruta—A System for Feature Selection, Fundamenta Informaticae, 2010; 101(4):271-285), incorporated by reference herein in its entirety, describes techniques for identifying features to be used as input to a machine learning model.

At act 256 of process 250, at least some of the total expression levels for genes associated with TME cells are included in the first set of features. For example, the total expression levels include those obtained at act 202 of process 200.

In some embodiments, all the obtained total expression levels for the genes associated with TME cells are included in the first set of features. In some embodiments, only a subset of the total expression levels is included in the first set of features. For example, in some embodiments, total expression levels for at least 10, at least 25, at least 30, at least 40, at least 50, at least 60, at least 75, at least 100, at least 150, at least 200, at least 250, at least 300, at least 350, at least 400 or all of the genes listed in Table 2 are included in the first set of features.

In some embodiments, the subset that is included in the first set of features depends on the type of cancer that the subject has or is suspected of having. For example, Table 3 lists genes associated with different types of cancer. For a patient having or suspected of having a particular type of cancer, total expression levels for genes associated with TME cells and associated with the type of cancer may be included in the first set of features.

In some embodiments, though not shown, generating the first set of features includes obtaining a first plurality of RNA percentages for cell types in the biological sample and including the first plurality of RNA percentages in the first set of features. For example, this may include obtaining a first RNA percentage for a TME cell of a first type and determining a second RNA percentage for a TME cell of a second type. Additionally or alternatively, this may include obtaining a second RNA percentage for tumor cells in the biological sample.

In some embodiments, RNA percentages are obtained using machine learning techniques. Example techniques for determining RNA percentages are described in the section “Cellular Deconvolution”. Some aspects of determining RNA percentages are also described in U.S. Patent Publication No. 2021-0287759, entitled “SYSTEMS AND METHODS FOR DECONVOLUTION OF EXPRESSION DATA”, the entire contents of which is herein incorporated by reference in its entirety.

In some embodiments, features to be included in the first set of features is identified as part of training the first machine learning model. Kursa et al. (Boruta—A System for Feature Selection, Fundamenta Informaticae, 2010; 101(4):271-285), incorporated by reference herein in its entirety, describes techniques for identifying features to be used as input to a machine learning model.

It should be appreciated that process 250 may include, in some embodiments, one or more additional acts for including one or more additional features in the first set of features, as aspects of the technology described herein are not limited in this respect. For example, generating the first set of features using process 250 may include obtaining and/or including one or more additional features to be included in the first set of features.

TABLE 4
Average expression profiles for genes associated with tumor cells.

| | | NK- | | | | B- | CD4+ | CD8+ | |
|---|---|---|---|---|---|---|---|---|---|
| Gene | Neutrophils | cells | Macrophages | Fibroblasts | Endothelium | cells | T-cells | T-cells | Monocytes |
| BCL2L1 | 24.95 | 76.6 | 68.31 | 93.21 | 111.3 | 53.58 | 69.47 | 44.73 | 21.13 |
| RRM2 | 1.57 | 33.38 | 10.16 | 33.63 | 49.59 | 111.2 | 51.94 | 9.34 | 1.07 |
| IGF2R | 342.95 | 83.07 | 117.69 | 77.39 | 36.48 | 42.06 | 28.41 | 51.35 | 66.36 |
| HDAC2 | 28.68 | 52.04 | 61.6 | 96.5 | 120.12 | 77.61 | 61.29 | 52.56 | 52.76 |
| BCL2L2 | 2.99 | 11.69 | 18.86 | 42.4 | 23.09 | 11.97 | 4.46 | 4.11 | 15.59 |
| CA9 | 0 | 0.03 | 0.01 | 1.01 | 0.03 | 0.01 | 0.47 | 0.05 | 0.01 |
| TP53 | 45.17 | 97.58 | 170.27 | 92.47 | 445.97 | 596.72 | 231.82 | 64.07 | 129.12 |
| AURKA | 3.83 | 12.48 | 10.59 | 32.88 | 33.83 | 42.54 | 25.92 | 7.89 | 4.43 |
| MKI67 | 0.52 | 10.88 | 4.9 | 14.94 | 28 | 62.15 | 24.37 | 5.6 | 0.68 |
| FGFR4 | 0.89 | 0.8 | 0.16 | 1.43 | 1.74 | 1.51 | 1.23 | 1.44 | 0.39 |
| EGF | 0.03 | 0.03 | 0.05 | 0.3 | 0.02 | 0.22 | 0.01 | 0.11 | 0.08 |
| CD22 | 9.76 | 3.33 | 14.72 | 0.24 | 0.21 | 245.04 | 1 | 2.39 | 3.67 |
| FLNA | 242.47 | 455.4 | 468.29 | 1123.48 | 743.71 | 257.11 | 303 | 456.78 | 469.93 |
| BIRC5 | 0.4 | 23.7 | 3.89 | 30.23 | 43.09 | 44.66 | 21.62 | 3.7 | 0.39 |
| CCNE1 | 0.35 | 2.57 | 4.13 | 9.96 | 8.12 | 26.9 | 12.37 | 3.86 | 1.19 |
| NF1 | 7.94 | 12.16 | 8.82 | 15.81 | 8.08 | 7.99 | 7.62 | 11.98 | 9.56 |
| HDAC9 | 2.3 | 8.72 | 8.82 | 8.24 | 7.46 | 23.45 | 2.64 | 4.43 | 36.3 |
| NF2 | 2.41 | 24.83 | 13.68 | 43.59 | 48.07 | 19.23 | 18.85 | 18.63 | 14.56 |
| AURKB | 1.97 | 29.36 | 4.6 | 24.59 | 41.99 | 104.85 | 37.79 | 7.44 | 1.9 |
| PLK1 | 0.56 | 14.35 | 5.9 | 38.44 | 53.06 | 70.48 | 24.37 | 4.15 | 0.7 |
| CHEK2 | 0.69 | 9.19 | 9.15 | 8.53 | 13.73 | 15.66 | 10.89 | 4.25 | 6.61 |
| TERT | 0 | 0.03 | 0 | 0 | 0.02 | 0.48 | 0.42 | 0.04 | 0.01 |
| STMN1 | 5.81 | 319.36 | 67.83 | 217.53 | 505.61 | 1076.48 | 238.12 | 124.58 | 4.82 |
| NAE1 | 6.98 | 61.55 | 23.04 | 49.99 | 55.65 | 59.7 | 67.67 | 70.64 | 14.65 |
| PDGFA | 1.63 | 3.72 | 8.44 | 18.77 | 48.09 | 3.99 | 6.29 | 7.4 | 3.61 |
| RRM1 | 0.58 | 28.21 | 13.34 | 50.88 | 40 | 53.85 | 46.02 | 19.81 | 5.3 |
| EPHA2 | 0.05 | 0.14 | 0.2 | 47.48 | 97.15 | 0.48 | 0.64 | 0.13 | 0.21 |
| HDAC1 | 38.89 | 141.53 | 49.27 | 61.18 | 87.94 | 134.4 | 110.28 | 126.1 | 75.99 |
| MAGEA2 | 0 | 0.03 | 0 | 0.01 | 0 | 0 | 0.02 | 0.03 | 0.0 |
| MAGEA12 | 0 | 0.06 | 0 | 0.06 | 0 | 0 | 0.01 | 0.02 | 0.01 |
| CDKN2A | 0.3 | 10.01 | 5.97 | 65.12 | 25.98 | 19.3 | 6.66 | 15.81 | 1.82 |
| BRCA1 | 10.22 | 12 | 5.59 | 8.93 | 12.35 | 34.58 | 18.04 | 7.98 | 7.49 |
| FGFR2 | 1.13 | 0.67 | 0.21 | 2.33 | 0.21 | 0.3 | 0.87 | 2.1 | 0.59 |
| FGFR3 | 0.04 | 0.2 | 0.18 | 0.95 | 1.03 | 0.16 | 0.15 | 0.24 | 0.18 |
| PTK7 | 0.66 | 2.12 | 0.36 | 150.76 | 28.97 | 1.67 | 2.16 | 4.36 | 0.63 |
| MYB | 1.35 | 2.33 | 0.42 | 0.39 | 0.18 | 16.2 | 11.91 | 4.08 | 2.19 |
| MAGEA3 | 0 | 0.1 | 0.01 | 0.01 | 0.07 | 0 | 0.1 | 0.07 | 0 |
| TYMS | 0.76 | 44.06 | 9.35 | 51.02 | 87.61 | 106.22 | 66.87 | 11.37 | 0.55 |
| DLL3 | 0.02 | 0.14 | 0.02 | 0.43 | 0.4 | 0.37 | 0.27 | 0.44 | 0.03 |
| ERBB3 | 1.33 | 1.35 | 0.33 | 3.03 | 0.57 | 0.55 | 2.27 | 4.23 | 1.29 |
| IGF1 | 0.21 | 0.38 | 23.3 | 4.78 | 1.66 | 7.94 | 1.18 | 0.48 | 0.1 |
| IGFIR | 33.77 | 15.67 | 5.46 | 19.18 | 21.9 | 2.23 | 13.2 | 8.48 | 7.76 |
| ADORA2B | 0.5 | 1.03 | 7.7 | 13 | 5.33 | 0.71 | 1.19 | 0.37 | 3.68 |
| TUBB3 | 0.12 | 0.85 | 9.5 | 141.43 | 147.52 | 1.71 | 2.2 | 0.78 | 0.27 |
| SMO | 0.03 | 0.1 | 0.17 | 11.47 | 6.37 | 1.72 | 0.22 | 0.07 | 0.05 |
| MAGEA1 | 0.01 | 0.01 | 0.01 | 0 | 0.01 | 0 | 0.01 | 0.02 | 0.01 |
| ROR2 | 0.02 | 0.06 | 0.43 | 8.28 | 0.06 | 0.11 | 0.12 | 0.54 | 0.02 |
| MAGEA4 | 0 | 0.32 | 0.01 | 0.03 | 0.01 | 0 | 0.02 | 0.03 | 0.05 |
| CDK2 | 5.96 | 22.94 | 7.15 | 27.86 | 28.6 | 43.92 | 26.99 | 17.17 | 4.9 |
| WT1 | 0.05 | 0.08 | 0.19 | 2.44 | 0.09 | 0.19 | 0.14 | 0.11 | 0.12 |
| ALK | 0.08 | 0.51 | 2.84 | 0.18 | 0.07 | 0.07 | 0.44 | 1.52 | 1.23 |
| MAGEA10 | 0.89 | 0.45 | 0.19 | 0.19 | 0.17 | 0.27 | 0.48 | 0.77 | 1.15 |
| CCND1 | 0.15 | 1.22 | 24.05 | 421.09 | 191.24 | 2.3 | 1.7 | 1.52 | 0.21 |
| PMEL | 0.41 | 0.78 | 0.83 | 12.42 | 1.24 | 1.64 | 3.33 | 3.53 | 1.27 |
| TXNRD1 | 170.03 | 68.5 | 290.48 | 569.53 | 447.44 | 81.49 | 64.29 | 53.51 | 58.97 |
| NOTCH3 | 0.45 | 0.19 | 7.53 | 44.11 | 1.6 | 0.14 | 0.23 | 0.45 | 0.77 |
| ERBB4 | 0.01 | 0.06 | 0.02 | 0.29 | 0.05 | 0.06 | 0.02 | 0.04 | 0.02 |
| NRAS | 10.85 | 42.14 | 48.38 | 38.24 | 59.37 | 48.2 | 33.9 | 34.62 | 53.26 |
| CDKN1A | 136.95 | 52.2 | 414.5 | 614.29 | 307.13 | 148.28 | 53.52 | 47.62 | 395.99 |
| FN1 | 2.92 | 4.95 | 509.09 | 10170.32 | 2260.78 | 0.38 | 8.91 | 0.85 | 4.56 |
| FLT1 | 5.34 | 1.48 | 13.81 | 7.01 | 94.75 | 5.39 | 3.68 | 2.57 | 2.13 |
| ERBB2 | 1.94 | 30.46 | 1.43 | 44.77 | 22.67 | 4.36 | 2.63 | 7.47 | 1.82 |
| MMP2 | 0.38 | 0.44 | 36.58 | 2546.94 | 860.71 | 0.05 | 1.82 | 0.48 | 0.27 |
| EPCAM | 0.23 | 0.44 | 0.15 | 0.26 | 0.25 | 0.06 | 0.19 | 0.44 | 0.01 |
| PGR | 0.01 | 0.02 | 0.01 | 0.38 | 55.28 | 0.01 | 0.01 | 0.01 | 0.01 |
| EGFR | 0.02 | 0.12 | 0.11 | 37.13 | 3.5 | 0.08 | 0.12 | 0.17 | 0.1 |
| ITGB4 | 3.58 | 1.05 | 0.71 | 2.93 | 25 | 0.93 | 1.05 | 3.1 | 0.62 |
| CDH1 | 0.19 | 0.37 | 0.54 | 1.54 | 0.09 | 2.58 | 0.89 | 1.67 | 0.14 |
| MUC1 | 0.75 | 1.11 | 2.09 | 18.44 | 1.48 | 1.42 | 5.2 | 2.89 | 1.08 |
| TPBG | 0.06 | 0.12 | 1.06 | 76.66 | 8.49 | 0.67 | 0.4 | 1.23 | 0.88 |
| TACSTD2 | 2.63 | 0.81 | 3.03 | 1.04 | 37.48 | 0.18 | 0.19 | 0.79 | 1.96 |
| AREG | 5.59 | 69.64 | 10.83 | 7.82 | 1.34 | 5.4 | 8.86 | 24.49 | 21.08 |
| CEACAM6 | 6.37 | 2.26 | 0.43 | 0.12 | 0.24 | 0.18 | 0.35 | 2.41 | 0.82 |
| SLC39A6 | 18.63 | 31.56 | 28.59 | 93.22 | 17.23 | 32.69 | 26.63 | 31.57 | 25.92 |
| CCND3 | 158.6 | 454.86 | 66.18 | 60.71 | 81.07 | 92.87 | 262.02 | 341.74 | 195.89 |
| CDK4 | 4.45 | 102.07 | 103.35 | 167.5 | 230.56 | 204.21 | 133.82 | 56.5 | 27.39 |
| KMT2E | 110 | 254.07 | 37.13 | 31.72 | 41.29 | 65.89 | 128.94 | 214.03 | 122.75 |
| RAD50 | 2.12 | 12.35 | 10.34 | 12.33 | 8.64 | 26.51 | 14.77 | 17.76 | 14.17 |
| MTOR | 8.24 | 24.84 | 16.32 | 19.2 | 25.45 | 26.06 | 20.19 | 26.3 | 18.75 |
| BRAF | 25.86 | 21.99 | 7.72 | 11.45 | 10.27 | 17.24 | 13.9 | 24.93 | 15.98 |
| CCNE2 | 3.38 | 8.09 | 3.24 | 5.44 | 9.58 | 14.29 | 10.56 | 6.38 | 2.61 |
| IGF2 | 0.05 | 0.11 | 0.45 | 102.29 | 28.49 | 0.12 | 0.69 | 0.68 | 0.05 |
| TOP1 | 71.92 | 37.84 | 46.53 | 57.25 | 66.73 | 100.3 | 48.04 | 45.33 | 49.31 |
| UMPS | 3.3 | 7.2 | 29.05 | 6.08 | 36.73 | 21.93 | 39.27 | 13.19 | 4.7 |
| CD274 | 31.73 | 6.5 | 43.69 | 6.33 | 14.62 | 18.81 | 8.41 | 7.5 | 0.89 |
| BRCA2 | 0.57 | 2.06 | 2.46 | 1.71 | 2.5 | 5.36 | 3.13 | 1.52 | 0.82 |
| ADORA2A | 159.12 | 13.05 | 29.81 | 3.59 | 20.46 | 38.63 | 23.96 | 37.36 | 13.4 |
| XRCC1 | 18.72 | 32.25 | 29.53 | 24.55 | 29.33 | 32.17 | 25.52 | 29.28 | 40.9 |
| TSC2 | 15.95 | 28.51 | 16.63 | 28.16 | 36.17 | 21.62 | 19.74 | 26.54 | 23.9 |
| INSR | 1.03 | 0.68 | 4.16 | 5.61 | 25.46 | 5.96 | 0.89 | 0.77 | 16.5 |
| ABCB1 | 1.44 | 54.99 | 0.46 | 0.78 | 6.8 | 1.97 | 4.69 | 44.73 | 0.12 |
| IDO1 | 36.51 | 7.02 | 161.51 | 2.4 | 3.03 | 1.16 | 1.03 | 0.7 | 1.63 |
| DPYD | 32.19 | 33.82 | 64.19 | 19.79 | 11.18 | 7.78 | 23.06 | 33.24 | 134.49 |
| BCL6 | 470.54 | 43.66 | 64.68 | 33.52 | 18.05 | 30.62 | 27.63 | 36.07 | 183.66 |
| FGFR1 | 2.24 | 9 | 19.49 | 123.62 | 78.75 | 6.24 | 10.02 | 16.25 | 4.5 |
| KRAS | 39.66 | 36.39 | 20.62 | 18.99 | 18.63 | 14.74 | 34.55 | 56.66 | 32.39 |
| MDM2 | 242.84 | 75.6 | 192.92 | 108.75 | 257.95 | 272.82 | 104 | 54.53 | 151.98 |
| IRF2 | 278.36 | 107.9 | 85.06 | 20.79 | 40.32 | 114.67 | 73.98 | 78.97 | 104.3 |
| AKT2 | 390.63 | 108.65 | 232.61 | 105 | 99.69 | 454.65 | 263.01 | 98.89 | 106.47 |
| XRCC5 | 97.21 | 174.39 | 102.87 | 160.52 | 188.94 | 200.55 | 180.69 | 165.83 | 132.63 |
| B2M | 1790.73 | 4693.28 | 468.59 | 373.95 | 158.37 | 891.56 | 2170.92 | 3534.44 | 1209.4 |
| KMT2C | 55.26 | 42 | 18.62 | 9.91 | 14.07 | 18.46 | 28.75 | 47.6 | 51.54 |
| HDAC4 | 20.89 | 32.47 | 11.19 | 7.86 | 9.72 | 5.44 | 18.02 | 22.99 | 31.26 |
| ICAM1 | 365.34 | 56.17 | 347.62 | 52.08 | 418.95 | 90.26 | 22.19 | 24.79 | 110.51 |
| NTRK3 | 0.23 | 0.18 | 0.12 | 1.47 | 0.12 | 0.11 | 0.96 | 0.46 | 0.32 |
| ATM | 23.2 | 160.21 | 18.76 | 14.59 | 11.95 | 31.24 | 94.02 | 181.53 | 55.42 |
| XRCC3 | 12.48 | 23.47 | 9.9 | 13.85 | 19.3 | 36.27 | 24.13 | 25.35 | 14.92 |
| ABCC3 | 0.54 | 0.65 | 22.63 | 7.4 | 2.08 | 0.8 | 0.48 | 1.03 | 9.32 |
| CCND2 | 6 | 110.59 | 5.54 | 8.01 | 8.58 | 87.95 | 107.83 | 85.89 | 10.61 |
| ROS1 | 0 | 0.02 | 0.03 | 0.38 | 0.02 | 0.02 | 0.04 | 0.02 | 0.03 |
| PTEN | 399.55 | 73.01 | 56.28 | 92.94 | 78.66 | 140.28 | 55.51 | 73.19 | 198.52 |
| SMARCA4 | 8.11 | 30.03 | 27.06 | 40.91 | 62.41 | 56.2 | 31.39 | 32.51 | 22.08 |
| ATF3 | 9.6 | 11.39 | 212.51 | 23.3 | 37.06 | 23.73 | 16.63 | 27.14 | 110.71 |
| RB1 | 16.78 | 20.33 | 52.22 | 28.81 | 24.53 | 49.27 | 17.28 | 21.03 | 39.72 |
| STK11 | 20.5 | 32.84 | 26.88 | 32.69 | 45.63 | 34.99 | 29.02 | 41.29 | 28.42 |
| ADORA1 | 0.09 | 0.05 | 0.18 | 3.18 | 0.03 | 0.01 | 0.03 | 0.03 | 0.31 |
| ERCC1 | 11.81 | 78.25 | 76.36 | 121.15 | 123.39 | 78.92 | 48.45 | 58.36 | 81.78 |
| PIK3CD | 191 | 146.54 | 30.07 | 10.16 | 5.21 | 81.88 | 93.13 | 139.37 | 78.36 |
| EREG | 6.29 | 1.49 | 40.4 | 4.03 | 0.14 | 0.67 | 1.05 | 1.2 | 47.13 |
| MCL1 | 1318.09 | 391.55 | 220.89 | 164.33 | 163.06 | 233.4 | 287.98 | 511.45 | 1220.38 |
| STAT6 | 454.59 | 150.87 | 167.5 | 91.05 | 118.17 | 214.99 | 146.56 | 127.96 | 312.29 |
| PIK3CG | 57.98 | 61.28 | 21.12 | 0.09 | 4.47 | 16.54 | 18.09 | 37.61 | 55.43 |
| ATR | 2.69 | 17.96 | 7 | 8.62 | 8.66 | 14.44 | 14.72 | 23.69 | 16.6 |
| CIITA | 5.81 | 13.73 | 24.19 | 0.33 | 1.99 | 89.05 | 4.12 | 7.36 | 61.11 |
| PDCD1LG2 | 1.23 | 0.55 | 16.62 | 28.59 | 13.93 | 5.38 | 0.95 | 0.69 | 0.59 |
| HDAC7 | 55.39 | 53.21 | 14.68 | 71.83 | 106.43 | 38.65 | 60.22 | 54.3 | 30.59 |
| PIK3CA | 26.78 | 17.86 | 11.93 | 13.67 | 16.49 | 11.63 | 22.12 | 26.33 | 21.62 |

FIG. 3A is a diagram of an illustrative technique 300 for estimating tumor expression levels of genes in tumor cells of a biological sample, according to some embodiments of the technology described herein.

As shown in FIG. 3A, a biological sample 301 is used to obtain expression data 303. The biological sample 301 includes tumor cells 301a and TME cells 301b. The TME cells 301b include TME cells of different types (e.g., Type A 322, Type B 324, and Type C 326). It should be appreciated that the number and types of TME cell populations shown in FIG. 3A are only illustrative, and a biological sample may include any suitable number and types of TME cell populations.

In some embodiments, the biological sample 301 is processed or may have been previously processed to obtain expression data 303. For example, the expression data may be generated using a sequencing platform (e.g., sequencing platform 102 shown in FIG. 1).

In some embodiments, the expression data 303 includes expression data for genes associated with tumor cells (also referred to herein as “tumor genes”) and genes associated with TME cells (also referred to herein as “TME genes”). In some embodiments, the tumor genes include a number of genes N and the TME genes include a number of genes M, which may be the same of different from N. For example, the tumor genes may include N genes listed in Table 2 and the TME genes may include M genes listed in Table 3. Additionally or alternatively, the N tumor genes may include at least 10 genes, at least 25 genes, at least 35 genes, at least 50 genes, at least 75 genes, at least 100 genes, at least 120 genes, between 10 and 130 genes, between 25 and 100 genes, between 50 and 100 genes, etc. The M TME genes may include at least 10 genes, at least 25 genes, at least 35 genes, at least 50 genes, at least 75 genes, at least 100 genes, at least 150 genes, at least 175 genes, at least 200 genes, at least 250 genes, at least 300 genes, at least 350 genes, at least 400 genes, at least 450 genes, between 10 and 475 genes, between 25 and 400 genes, between 50 and 350 genes, between 100 and 300 genes, etc.

In some embodiments, the expression data 303 includes the total expression level for each of the listed tumor genes and each of the listed TME genes. For example, the expression data 303 includes the total expression level for a first gene associated with tumor cells and the total expression level for a first gene associated with TME cells.

In some embodiments, the expression data 303 is used to generate a set of features for each of the genes associated with tumor cells. For example, the expression data 303 is used to generate a first set of features 304a for the first tumor gene, a second set of features 304b for the second tumor gene, and an M^{th} set of features 304c for the M^{th} tumor gene. In some embodiments, all of the expression data 303 is used to generate a set of features for a gene. Additionally or alternatively, only a subset of the expression data (e.g., only a subset of the total expression levels of the tumor genes and/or TME genes) is used to generate a set of features for a gene. Example techniques for generating a set of features for a gene are described herein including at least with respect to FIG. 2C. Example sets of features for a gene are described herein including at least with respect to FIG. 3B.

In some embodiments, each set of features is provided as input to a respective machine learning model to obtain a corresponding output. For example, the first set of features 304a is provided as input to a first machine learning model 306a to obtain an output 308a indicative of the TME expression level estimate of the first gene in TME cells 301b of the biological sample 301. The second set of features 304b is provided as input to a second machine learning model 306b to obtain an output 308b indicative of the TME expression level estimate of the second gene in TME cells 301b of the biological sample. The M^{th} set of features is provided as input to an M^{th} machine learning model 306c to obtain an output 308c indicative of the TME expression level estimate of the M^{th} gene in TME cells 301b of the biological sample. Example techniques for using a machine learning model to obtain an output indicative of a TME expression level estimate of a gene are described herein including at least with respect to act 224 of process 220 shown in FIG. 2B.

In some embodiments, the output of each machine learning model is used to determine a tumor expression level estimate of the gene. For example, the output 308a of the first machine learning model 306a is used to determine the tumor expression level 310a for the first gene in the tumor cells 301a of the biological sample 301. The output 308b of the second machine learning model 306b is used to determine the tumor expression level 310b for the second gene in the tumor cells 301b of the biological sample 301. The output 308c of the M^{th} machine learning model 306c is used to determine the tumor expression level 310c for the M^{th} gene in the tumor cells 301c of the biological sample 301. Example techniques for using the output of a machine learning model to determine the tumor expression level of a gene are described herein including at least with respect to act 226 of process 220 shown in FIG. 2B.

FIG. 3B is a diagram depicting an illustrative example of sets of features generated for the genes in the tumor cells of the biological sample, according to some embodiments of the technology described herein.

As shown in FIG. 3B, the expression data 303 is used to generate M sets of features for M genes associated with tumor cells of a biological sample, including a first set of features 304a for a first gene, a second set of features 304b for a second gene, and an M^{th} set of features 304c for an M^{th} gene.

In some embodiments, the first set of features 304a includes any suitable features for the first gene including, for example, an initial expression level estimate 352a for the first gene, at least some of the total expression levels 354a for the tumor genes, at least some of the total expression levels 356a for the TME genes, and/or a first plurality of RNA percentages 358a. It should be appreciated that the first set of features 304a may include additional or fewer features than those shown in FIG. 3B, as aspects of the technology are not limited in this respect.

In some embodiments, the initial expression level estimate 352a may be based on (a) the total expression level for the first gene in the biological sample, (b) RNA percentages for the TME cell populations 301b (e.g., RNA percentages for TME cell populations of Type A 322, Type B 324, and Type C 326), and (c) average expression levels of the first gene in each of the TME cell populations. Example techniques for determining an initial expression level estimate are described herein including at least with respect to act 252 of process 250, shown in FIG. 2C.

In some embodiments, the total expression levels 354a for the tumor genes include all or a subset of the total expression levels included in the expression data 303 for genes 1-M. For example, the subset of the total expression levels may be selected based on a type of cancer that the subject has or is suspected of having. Example techniques for identifying the total expression levels for tumor genes to be included in a set of features are described herein including at least with respect to act 254 of process 250, shown in FIG. 2C.

In some embodiments, the total expression levels 356a for the TME genes include all or a subset of the total expression levels included in the expression data 303 for genes 1-N. For example, the subset of the total expression levels may be selected based on a type of cancer that the subject has or is suspected of having. Example techniques for identifying the total expression levels for TME genes to be included in a set of features are described herein including at least with respect to act 256 of process 250, shown in FIG. 2C.

In some embodiments, the first plurality of RNA percentages 358a include RNA percentages for each of multiple cell types in the biological sample. In some embodiments, each of the first plurality of RNA percentages 358a is indicative of the percent of RNA sequence reads that have aligned to the first gene that originate from a particular cell type in the biological sample. For example, the first plurality of RNA percentages may include a first RNA percentage indicative of the percentage of RNA sequence reads that have aligned to the first gene that originate from the first cell type. The first plurality of RNA percentages 358a may include RNA percentages for one or more TME population of different cell types and/or an RNA percentage for tumor cells in the biological sample.

In some embodiments, the second set of features 304b includes any suitable features for the second gene including, for example, an initial expression level estimate 352b for the second gene, at least some of the total expression levels 354b for the tumor genes, at least some of the total expression levels 356b for the TME genes, and/or a second plurality of RNA percentages 358b. It should be appreciated that the second set of features 304b may include additional or fewer features than those shown in FIG. 3B, as aspects of the technology are not limited in this respect. It should be appreciated that the second set of features 304b may be different from the first set of features (e.g., completely or partially different) or identical to the first set of features 304a, as aspects of the technology described herein are not limited in this respect.

In some embodiments, the initial expression level estimate 352b may be based on (a) the total expression level for the second gene in the biological sample, (b) RNA percentages for the TME cell populations 301b (e.g., RNA percentages for TME cell populations of Type A 322, Type B 324, and Type C 326), and (c) average expression levels of the second gene in each of the TME cell populations. Example techniques for determining an initial expression level estimate are described herein including at least with respect to act 252 of process 250, shown in FIG. 2C.

In some embodiments, the total expression levels 354b for the tumor genes include all or a subset of the total expression levels included in the expression data 303 for genes 1-M. For example, the subset of the total expression levels may be selected based on a type of cancer that the subject has or is suspected of having. Example techniques for identifying the total expression levels for tumor genes to be included in a set of features are described herein including at least with respect to act 254 of process 250, shown in FIG. 2C.

In some embodiments, the total expression levels 356b for the TME genes include all or a subset of the total expression levels included in the expression data 303 for genes 1-N. For example, the subset of the total expression levels may be selected based on a type of cancer that the subject has or is suspected of having. Example techniques for identifying the total expression levels for TME genes to be included in a set of features are described herein including at least with respect to act 256 of process 250, shown in FIG. 2C.

In some embodiments, the second plurality of RNA percentages 358b include RNA percentages for each of multiple cell types in the biological sample. In some embodiments, each of the second plurality of RNA percentages 358b is indicative of the percent of RNA sequence reads that have aligned to the second gene that originate from a particular cell type in the biological sample. For example, the second plurality of RNA percentages may include a first RNA percentage indicative of the percentage of RNA sequence reads that have aligned to the second gene that originate from the first cell type. The first plurality of RNA percentages 358b may include RNA percentages for one or more TME population of different cell types and/or an RNA percentage for tumor cells in the biological sample.

In some embodiments, the M^{th} set of features 304c includes any suitable features for the M^{th} gene including, for example, an initial expression level estimate 352c for the M^{th} gene, at least some of the total expression levels 354c for the tumor genes, at least some of the total expression levels 356c for the TME genes, and/or an M^{th} plurality of RNA percentages 358c. It should be appreciated that the M^{th} set of features 304c may include additional or fewer features than those shown in FIG. 3B, as aspects of the technology are not limited in this respect. It should be appreciated that the M^{th} set of features 304c may be different (e.g., completely or partially different) from the first set of features 304a and/or the second set of features or identical to the first set of features 304a and or the second set of features 304b, as aspects of the technology described herein are not limited in this respect.

In some embodiments, the initial expression level estimate 352c may be based on (a) the total expression level for the M^{th} gene in the biological sample, (b) RNA percentages for the TME cell populations 301b (e.g., RNA percentages for TME cell populations of Type A 322, Type B 324, and Type C 326), and (c) average expression levels of the first gene in each of the TME cell populations. Example techniques for determining an initial expression level estimate are described herein including at least with respect to act 252 of process 250, shown in FIG. 2C.

In some embodiments, the total expression levels 354c for the tumor genes include all or a subset of the total expression levels included in the expression data 303 for genes 1-M. For example, the subset of the total expression levels may be selected based on a type of cancer that the subject has or is suspected of having. Example techniques for identifying the total expression levels for tumor genes to be included in a set of features are described herein including at least with respect to act 254 of process 250, shown in FIG. 2C.

In some embodiments, the total expression levels 356c for the TME genes include all or a subset of the total expression levels included in the expression data 303 for genes 1-N. For example, the subset of the total expression levels may be selected based on a type of cancer that the subject has or is suspected of having. Example techniques for identifying the total expression levels for TME genes to be included in a set of features are described herein including at least with respect to act 256 of process 250, shown in FIG. 2C.

In some embodiments, the M^{th} plurality of RNA percentages 358c include RNA percentages for each of multiple cell types in the biological sample. In some embodiments, each of the M^{th} plurality of RNA percentages 358c is indicative of the percent of RNA sequence reads that have aligned to the M^{th} gene that originate from a particular cell type in the biological sample. For example, the M^{th} plurality of RNA percentages may include a first RNA percentage indicative of the percentage of RNA sequence reads that have aligned to the M^{th} gene that originate from the first cell type. The M^{th} plurality of RNA percentages 358c may include RNA percentages for one or more TME population of different cell types and/or an RNA percentage for tumor cells in the biological sample

FIG. 4 is a block diagram of a system 400 including example computing device 404 and software 410, according to some embodiments of the technology described herein.

In some embodiments, computing device 404 includes software 410 configured to perform various functions with respect to the expression data (e.g., expression data 103 shown in FIG. 1). In some embodiments, software 410 includes a plurality of modules. A module may include processor-executable instructions that, when executed by at least one computer hardware processor, cause the at least one computer hardware processor to perform the function(s) of the module. Such modules are sometimes referred to herein as “software modules.” each of which includes processor executable instructions configured to perform one or more processes, such as the processes described herein including at least with respect to FIGS. 2A-2C and FIG. 6.

For example, as shown in FIG. 4, software 410 includes one or more software modules for processing expression data, such as feature generation module 460, expression level determination module 462 and RNA percentage determination module 464. In some embodiments, the software 410 additionally includes a user interface module 458, a sequencing platform interface module 448, and/or a data store interface module 442 for obtaining data (e.g., user input, expression data, machine learning model(s)). In some embodiments, data is obtained from sequencing platform 444, expression data store 446, and/or machine learning model data store 454. In some embodiments, the software 410 further includes machine learning model training module 452 for training one or more machine learning models (e.g., stored in machine learning model data store 454).

In some embodiments, the feature generation module 460 obtains expression data from the expression data store 446 and/or the sequencing platform 444.

In some embodiments, the feature generation module 460 generates sets of features for respective genes of a set of genes associated with tumor cells (e.g., genes listed in Table 1). For example, the feature generation module 460 may generate a first set of features for a first gene listed in Table 1.

In some embodiments, a set of features generated by the feature generation module 460 includes at least some of the obtained expression data and an initial expression level estimate of a gene in tumor cells of a biological sample. However, it should be appreciated that other information may be included in the set of features.

In some embodiments, the expression data included in the set of features includes total expression levels for genes associated with tumor cells in a biological sample and total expression levels for genes associated with TME cells in the biological sample. For example, the set of features may include a first total expression level for a first gene associated with tumor cells (e.g., genes listed in Table 1) and/or a second total expression level for a second gene associated with TME cells (e.g., genes listed in Table 2).

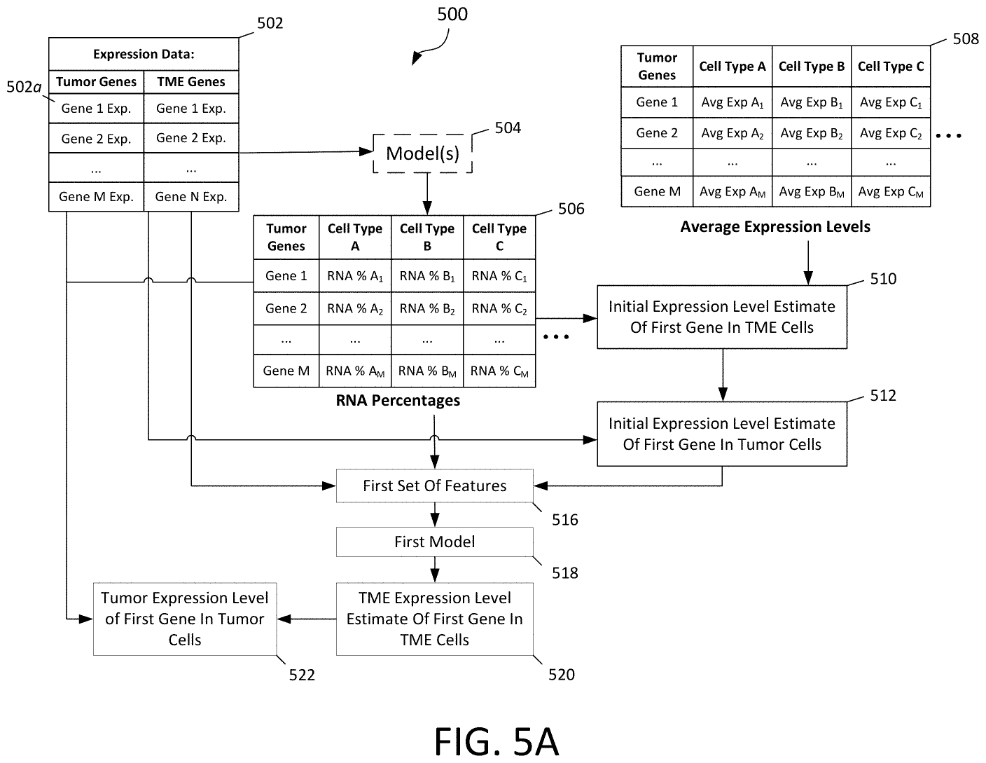

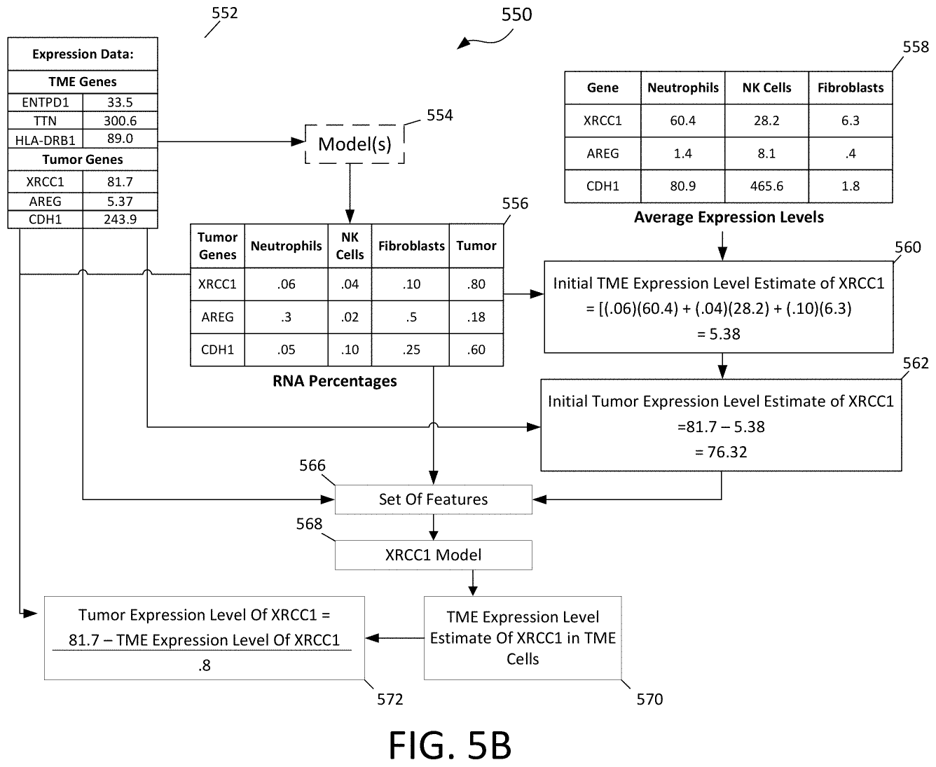

In some embodiments, the initial expression level estimate of a gene is determined using the feature generation module 460. In some embodiments, determining the initial expression level estimate for a gene includes obtaining average expression levels for the gene in multiple TME cell populations and obtaining RNA percentages for the multiple TME cell populations in the biological sample. For example, the average expression levels may be obtained from the expression data store 446 via the data store interface module 442 and the RNA percentages may be obtained from the cell composition determination module 464. In some embodiments, the feature generation module 460 determines an initial expression level estimate for a gene based on the average expression levels of a gene, the corresponding RNA percentages, and the total expression level of the gene in the biological sample. Techniques for determining an initial expression level estimate are described herein including at least with respect to FIG. 2C and FIGS. 5A-5B.

In some embodiments, cell composition determination module 464 obtains expression data from sequencing platform 444 and/or expression data 446. In some embodiments, the obtained expression data includes total expression levels for genes associated with tumor and TME cells in a biological sample.

In some embodiments, the cell composition determination module 464 processes the obtained expression data to determine one or more RNA percentages for a biological sample. For example, the cell composition determination module 464 may process the expression data to determine RNA percentages for tumor cells in a biological sample. Additionally or alternatively, the cell composition determination module 464 may process the expression data to determine RNA percentages for TME cells of different types in the biological sample. As nonlimiting examples, the cell composition determination module 464 may determine, for a particular gene, an RNA percentage for neutrophils in the TME and an RNA percentage for B cells in the TME. Techniques for determining RNA percentages are described herein including at least with respect to FIGS. 2A-2C.

In some embodiments, the expression level determination module 462 obtains sets of features from the feature generation module 460, obtains machine learning models from the machine learning model data store 454, and obtains RNA percentages from the RNA percentage determination module 464.

In some embodiments, the obtained machine learning models include a machine learning model for each of multiple genes associated with tumor cells (e.g., genes listed in Table 1). For example, the machine learning models may include a first machine learning model for a first gene listed in Table 1. In some embodiments, the machine learning models may each be trained to estimate a TME expression level of a gene in TME cells of a biological sample. For example, the first machine learning model may be trained to estimate the TME expression of the first gene in TME cells of the biological sample.

In some embodiments, the obtained RNA percentage include an RNA percentage for tumor cells in the biological sample. In some embodiments, the RNA percentage indicates a percent of RNA sequence reads that have aligned a particular gene that originate from tumor cells in the biological sample.

In some embodiments, the expression level determination module 462 processes the obtained features using the machine learning models to determine estimate TME expression levels of genes in TME cells of a biological sample. For example, the expression level determination module 462 may process a first set of features generated for a first gene using a first machine learning model to obtain an output indicative of an estimate TME expression level of the first gene in TME cells of the biological sample. In some embodiments, the expression level determination module 462 may use a different machine learning model to process each set of features (e.g., corresponding to different genes associated with tumor cells).

In some embodiments, the expression level determination module 462 determines tumor expression levels for genes associated with tumor cells based on the outputs of the machine learning models, the obtained RNA percentage for tumor cells in the biological sample, and total expression levels for the genes in the biological sample. For example, the expression level determination module 462 may determine a first tumor expression level for a first gene based on an output of a first machine learning model, the RNA percentage for the tumor cells, and the total expression level of the first gene in the biological sample. Techniques for determining tumor expression levels are described herein including at least with respect to FIGS. 2A-2C, FIGS. 3A-3B and FIGS. 5A-5B.

In some embodiments, the feature generation module 460 and the cell composition determination module 464 obtain the expression data and/or average expression levels via one or more interface modules. In some embodiments, the interface modules include sequencing platform interface module 448 and data store interface module 442. The sequencing platform interface module 448 may be configured to obtain (either pull or be provided) expression data from the sequencing platform 444. The data store interface module 442 may be configured to obtain (either pull or be provided) expression data and/or the average expression levels from the expression data store 446. The data may be provided via a communication network (not shown), such as Internet or any other suitable network, as aspects of the technology described herein are not limited to any particular communication network.

In some embodiments, the expression data store 446 includes any suitable data store, such as a flat file, a data store, a multi-file, or data storage of any suitable type, as aspects of the technology described herein are not limited to any particular type of data store. The expression data store 446 may be part of software 404 (not shown) or excluded from software 404, as shown in FIG. 4.

In some embodiments, expression data store 446 stores expression data obtained from biological sample(s) of one or more subjects. In some embodiments, the expression data may be obtained from sequencing platform 444 and/or from one or more public data stores and/or studies. In some embodiments, a portion of the expression data may be processed by the feature generation module 460 to generates sets of features to be provided as input to machine learning models. In some embodiments, a portion of the expression data may be processed by the cell composition determination module 464 to determine RNA percentages for cell populations in a biological sample. In some embodiments, a portion of the expression data may be processed by the expression level determination module 462 to determine tumor expression levels of genes in tumor cells of a biological sample. In some embodiments, a portion of the expression data may be used to train one or more machine learning models (e.g., with the machine learning classifier training module 464).

In some embodiments, the expression level determination module 462 obtains the machine learning models via the data store interface module 442. The data store interface module 442 may be configured to obtain (either pull or be provided) machine learning models from the machine learning model data store 454. The machine learning models may be provided via a communication network (not shown), such as Internet or any other suitable network, as aspects of the technology described herein are not limited to any particular communication network.

In some embodiments, machine learning classifier data store 454 includes any suitable data store, such as a flat file, a data store, a multi-file, or data storage of any suitable type, as aspects of the technology described herein are not limited to any particular type of data store. The machine learning classifier data store 454 may be part of software 404 (not shown) or excluded from software 410, as shown in FIG. 4.

In some embodiments, the machine learning model data store 454 stores a plurality of machine learning models used to determine TME expression level estimates for genes in TME cells of a biological sample. In some embodiments, each machine learning model corresponding to a gene of a set of genes associated with tumor cells (e.g., genes listed in Table 1).

In some embodiments, machine learning model training module 452, referred to herein as training module 452, is configured to train the one or more machine learning models used to estimate TME expression levels for genes in TME cells of the biological sample. This may include training a first machine learning model to estimate a TME expression level for a first gene in TME cells of a biological sample. In some embodiments, the training module 452 trains a machine learning model using a training set of expression data. For example, the training module 452 may obtain training data via data store interface module 442. In some embodiments, the training module 452 may provide trained machine learning models to the machine learning model data store 454 via data store interface module 442. Techniques for training machine learning models are described herein including at least with respect to FIG. 6.

In some embodiments, the determined tumor expression levels may be output from the expression level determination module 462. For example, the tumor expression level estimates may be output to a user 456 via user interface 458. Additionally or alternatively, the determined tumor expression levels may be stored in memory.

User interface 448 may be a graphical user interface (GUI), a text-based user interface, and/or any other suitable type of interface through which a user may provide input. For example, in some embodiments, the user interface may be a webpage or web application accessible through an Internet browser. In some embodiments, the user interface may be a graphical user interface (GUI) of an app executing on the user's mobile device. In some embodiments, the user interface may include a number of selectable elements through which a user may interact. For example, the user interface may include dropdown lists, checkboxes, text fields, or any other suitable element.

FIG. 5A and FIG. 5B depict illustrative examples for estimating a tumor expression level of a gene in tumor cells of a biological sample, according to some embodiments of the technology described herein.

As shown in FIG. 5A, expression data 502 includes total expression levels for genes associated with tumor cells (e.g., genes 1-M) and total expression levels for genes associated with TME cells (e.g., genes 1-N). For example, the expression data 502 includes a total expression level for a first gene associated with tumor cells and a total expression level for a first gene associated with TME cells.

In some embodiments, the expression data 502 is used to obtain, for different genes (e.g., genes 1-M) RNA percentages 506 for different cell populations in the biological sample. In some embodiments, the expression data 502 is processed using one or more machine learning models 504 to obtain the RNA percentages 506. For example, the expression data 502 may be processed using the techniques described herein including at least with respect to FIG. 2B and the section “Cellular Deconvolution”.

In some embodiments, the RNA percentages 506 include RNA percentages for tumor cells and for TME cells of different types. For example, the RNA percentages include an RNA percentage for TME cells of Type A, an RNA percentage for TME cells of Type B, and an RNA percentage of TME cells of Type C. It should be appreciated that this is meant to be an illustrative example, and any suitable number of RNA percentages corresponding to any suitable number of cell populations in the biological sample may be included in RNA percentages 506.

The average expression levels 508 include the average expression levels of genes associated with tumor cells (e.g., genes 1-M) in each of multiple different cell types (e.g., TME cell types). For example, average expression levels for genes 1-M in TME cells of Type A, TME cells of Type B, and TME cells of Type C. In some embodiments, as described herein including at least with respect to FIG. 2C, the average expression level of a particular gene in a particular cell population represents the average expression level of that gene in that cell population across multiple biological samples and/or training samples.

In some embodiments, the average expression levels 508 and the RNA percentages 506 are used to generate an initial expression level estimate 510 of the first gene in TME cells of the biological sample. For example, in some embodiments, this may include determining a weighted sum using the average expression levels 508 for the first gene in the different TME cell populations (e.g., Type A, Type B, and Type C) and the corresponding RNA percentages for those cell populations. For example, determining the initial expression level estimate 510 of the first gene in the TME cells may include using Equation 3.

In some embodiments, the expression data 502 and the initial expression level estimate 510 of the first gene in the TME cells are used to determine the initial expression level estimate 512 of the first gene in the tumor cells of the biological sample. For example, in some embodiments, the initial expression level estimate 510 of the first gene in the TME cells of the biological sample is subtracted from the total expression level 502a of the first gene in the biological sample. For example, determining the initial expression level estimate 510 of the first gene in the tumor cells may include using Equation 4.

In some embodiments, the initial expression level estimate 512 of the first gene in the tumor cells and at least some of the expression data 502 are included in the first set of features 516. For example, at least a subset (e.g., some or all) of the total expression levels for the genes associated with tumor cells (e.g., total expression level 502a) and at least a subset of the total expression levels for the genes associated with TME cells are included in the first set of features 516.

Additionally or alternatively, the RNA percentages 506 are included in the first set of features 516. For example, at least a subset (e.g., some or all) of the RNA percentages 506 are included in the first set of features 516.

In some embodiments, the first set of features 516 is provided as input to the first machine learning model 518 to obtain an output 520 indicative of the TME expression level estimate of the first gene in TME cells of the biological sample.

In some embodiments, the output 520, at least some of the expression data 502, and one or more of the RNA percentages 506 are used to determine the tumor expression level of the first gene in the tumor cells of the biological sample. For example, the TME expression level estimate may be subtracted from the total expression level 502a of the first gene in the biological sample. The difference may, in some embodiments, be divided by the RNA percentage of tumor cells in the biological sample to obtain the tumor expression level 522. For example, determining the tumor expression level 522 for the first gene may include using Equations 1 and 2.

FIG. 5B depicts an illustrative example for estimating a tumor expression level of the XRCC1 gene in tumor cells of a biological sample.

As shown in FIG. 5B, expression data 552 is obtained for a biological sample. The expression data 552 includes expression data for genes associated with TME cells (e.g., the ENTPD1, TTN, and HLA-DRB1 genes) and expression data for genes associated with tumor cells (e.g., the XRCC1, AREG, and CDH1 genes). For example, the expression data for genes associated with TME cells includes total expression levels for each of the genes associated with TME cells. The expression data for genes associated with tumor cells includes total expression levels for each of the genes associated with tumor cells, including a total expression level for the XCC1 gene (81.7).

In some embodiments, the expression data 552 is used to obtain the RNA percentages 556 for different cell populations in the biological sample. In some embodiments, this includes processing the expression data using a machine learning model to obtain the RNA percentages 556, as described herein including at least with respect to FIG. 5A.

In some embodiments, the RNA percentages 556 includes an RNA percentage for the tumor cells and for TME cell populations in the biological samples. For the purpose of this example, the biological sample includes tumor cells and TME cells including neutrophils, NK cells, and fibroblasts. The RNA percentages 556 are indicative of a percent of RNA sequence reads aligned to the respective gene (e.g., XRCC1, AREG, CDH1, etc.) that originated from a respective cell population (e.g., neutrophils, NK cells, fibroblasts, tumor cells, etc.) In this example, for the XRCC1 gene, 6% of the RNA sequence reads that aligned to the XRCC1 gene originated from neutrophils, 4% originated from NK cells, 10% originated from fibroblasts, and 80% originated from tumor cells.

In some embodiments, average expression levels 558 are obtained for each gene associated with tumor cells in different cell population in the biological sample. For example, for the XRCC1 gene, the average expression levels 558 include an average expression level of the XRCC1 gene in each of the TME cell populations (e.g., the neutrophils, NK cells, and fibroblasts) in the biological sample.

In some embodiments, the RNA percentages 556 and the average expression levels 558 are used to determine an initial TME expression level estimate 560 of XRCC1. As shown in FIG. 5B, the initial TME expression level estimate 560 is determined by determining a weighted sum using the RNA percentages 556 and the average expression levels 558 for the XRCC1 gene. In particular, in the example, the weighted sum is determined by multiplying the average expression of the XRCC1 gene in a particular cell type with the corresponding RNA percentage for the cell type (e.g., using Equation 3). For example, the RNA percentage for neutrophils (0.06) is multiplied by the average expression of the XRCC1 gene in neutrophils (60.4).

In some embodiments, at least some of the expression data 552 and the initial TME expression level estimate 560 of the XRCC1 gene are used to determine the initial tumor expression level estimate 562 of the XRCC1 gene. For example, as shown, the initial TME expression level estimate 560 of the XRCC1 gene (5.38) may be subtracted from the total expression level of the XRCC1 gene (81.7) in the biological sample to obtain the initial tumor expression level estimate 562 of the XRCC1 gene (72.8).

In some embodiments, at least some of the expression data 552, at least some of the RNA percentages 556, and the initial tumor expression level estimate 562 are included in the set of features 566 for the XRCC1 gene. For example, the expression data 552 included in the set of features 566 may include all of the total expression levels for the tumor genes and/or all of the total expression levels for the TME genes. Additionally or alternatively, the expression data 552 included in the set of features 566 may include only a subset of the total expression levels for the tumor genes (e.g., including the total expression level for the XRCC1 gene) and/or only a subset of the total expression levels for the TME genes.

In some embodiments, the set of features 566 is provided as input to a machine learning model 568 for the XRCC1 gene to obtain an output 570 indicative of the TME expression level estimate of XRCC1 in the TME cells of the biological sample. For example, the TME expression level estimate may indicate an estimated expression of XRCC1 in the TME cells of the biological sample.

In some embodiments, the output 570, expression data 552, and RNA percentages 556 are used to determine the tumor expression level 572 of the XRCC1 gene in tumor cells of the biological sample. In some embodiments, as shown, determining the tumor expression level 572 includes subtracting the TME expression level estimate of the XRCC1 gene from the total expression level of the XRCC1 gene in the biological sample (81.7) and dividing the difference by the RNA percentage of tumor cells (0.80) in the biological sample. For example, as shown, the TME expression level of the XRCC1 gene is subtracted from 81.7 and divided by 0.80 to obtain the tumor expression level of the XRCC1 gene.

Machine Learning Model Training

FIG. 6 is a flowchart depicting a process 600 for training a machine learning model (e.g., the first machine learning models described herein including at least with respect to FIG. 2B) to estimate a tumor microenvironment (TME) expression level of a gene in TME cells of a biological sample, according to some embodiments of the technology described herein. In some embodiments, process 600 may be repeated to train each of a plurality of machine learning models to obtain a TME expression level for each of a respective plurality of genes.

Process 600 may be performed by any suitable computing device(s). For example, process 600 may be performed by a laptop computer, a desktop computer, one or more servers, in a cloud computing environment, computing device 2400 as described herein within respect to FIG. 24, or in any other suitable way. In some embodiments, process 600 may be performed using a software module on a computing device, such as the machine learning model training module 452 described herein including at least with respect to FIG. 4.

Process 600 begins at act 602 where training data is obtained. In some embodiments, the training data includes simulated expression data associated with one or more training samples (e.g., biological samples). In some embodiments, the simulated expression data may include expression data that is generated partially in silico. For example, the simulated expression data may include data that was obtained by sampling reads from multiple expression data sets from purified cell type samples. In some embodiments, the simulated expression data may comprise expression data measured in TPM. For example, the simulated expression data includes simulated expression data for genes associated with tumor cells and simulated expression data for genes associated with TME cells. For example, genes associated with tumor cells may include genes listed in Table 1 and the gene associated with TME cells may include genes listed in Table 2.

In some embodiments, the training data includes simulated expression data for genes associated with tumor cells and simulated expression data for genes associated with TME cells. For example, genes associated with tumor cells may include genes listed in Table 1 and the gene associated with TME cells may include genes listed in Table 2. In some embodiments, the simulated expression data for the genes associated with tumor cells includes total expression levels for the genes in the training sample(s). For example, the simulated expression data may include a first total expression level for a first gene associated with tumor cells. In some embodiments, the simulated expression data for the genes associated with TME cells includes total expression levels for genes in the training sample(s). For example, the simulated expression data may include a second total expression level for a second gene associated with TME cells.

In some embodiments, the training data may be generated as part of act 602. As described herein including at least with respect to FIG. 7A, in some embodiments the simulated expression data may be generated by combining expression data from tumor cells (e.g., cancer cells) with expression data from TME cells (e.g., immune cells, skin cells, etc.) to produce a plurality of simulated mixtures (which may be referred to herein as “artificial mixtures” or “mixes”) for training. In some embodiments, at least a thousand, at least ten thousand, at least one hundred thousand, or at least one million mixes may be generated and/or accessed as part of act 602.

The training data may be obtained in any suitable manner at act 602. For example, the training data may be stored on at least one storage medium (e.g., in one or more files, or in a database). In some embodiments, the at least one storage medium storing the training data may be local to the computing device (e.g., stored on the same at least one non-transitory storage medium), or may be external to the computing device (e.g., stored in a remote database or a cloud storage environment). The training data may be stored on a single storage medium, or may be distributed across multiple storage mediums.

In some embodiments, act 602 may further comprise pre-processing the training data in any suitable manner. For example, the training data may be sorted, combined, organized into batches, filtered, or pre-processed with any other suitable techniques. The pre-processing may make the training data suitable to be processed using the one or more machine learning models, for example. In some embodiments, the training data may be split into separate training, validation, and holdout datasets.

At act 604, generating a training set of features is formed using the training data. In some embodiments, generating the training set of features includes obtaining an initial expression level estimate of the gene in the tumor cells of the training sample(s). The initial expression level estimate may be included in the training set of features. In some embodiments, generating the training set of features includes including, in the training set of features, at least some of the total expression levels for genes associated with tumor cells and at least some of the total expression levels for genes associated with TME cells. For example, the total expression levels may include the total expression levels obtained at act 602. In some embodiments, generating the training set of features includes including, in the training set of features, RNA percentages obtained for the biological sample. Techniques for generating features are further described herein including at least with respect to FIG. 2C.

At act 606, a first machine learning model is trained to estimate a TME expression level of a first gene in TME cells of the training sample(s). In some embodiments, at sub-act 606a, the training set of features may be provided as input to a first machine learning model (e.g., the first machine learning model described herein including with respect to FIG. 2B). In some embodiments, other inputs may be additionally or alternatively be provided as input to the first machine learning model. The first machine learning model outputs, in some embodiments, an estimate of the TME expression level of the first gene in the TME cells of the training sample(s).

At sub-act 606b, training the first machine learning model may proceed with updating parameters using the estimate of the TME expression level output at sub-act 606a. In some embodiments, the estimate of the TME expression level may be compared to a known value for the TME expression level of the first gene in the TME cells as part of sub-act 606b. For example, a loss function may be applied to the estimated value and the known value in order to determine a loss associated with the estimated value. In some embodiments, the loss may be used to update the parameters of the model. For example, a gradient descent, or any other suitable optimization technique, may be applied in order to update the parameters of the model so as to minimize the loss.

The first machine learning model may process its input using any suitable techniques, as described herein. In some embodiments, the first model may use a gradient boosting machine learning technique. For example, the first model may comprise an ensemble of weak prediction models, such as decision trees, or any other suitable prediction models, which may be combined in an iterative fashion using a gradient boosting algorithm. In some embodiments, a gradient boosting framework such as XGBoost, LightGBM, Catboost, or Adaboost may be used as part of training the first model.

In some embodiments, for a given machine learning model, sub-acts 606a and 606b may be repeated multiple times (e.g., at least one hundred, at least one thousand, at least ten thousand, at least one hundred thousand, or at least one million times). In some embodiments, sub-acts 606a and 606b may be repeated for a set number of iterations or may be repeated until a threshold is surpassed (e.g., until loss decreases below a threshold value).

At act 608, process 600 proceeds with determining whether there are additional machine learning models to be training. For example, the plurality of machine learning models may include a second machine learning model for a second gene associated with tumor cells. Acts 602-606 may be repeated to train the second machine learning model to estimate the TME expression level of the second gene in the TME cells of the training sample(s). Additionally or alternatively, the plurality of machine learning models may include a third machine learning model for a third genes associated with tumor cells. Acts 602-606 may be repeated to train the third machine learning model to estimate the TME expression level of the third gene in the TME cells of the training sample(s).

If there are no remaining machine learning models to be trained, in some embodiments, the trained plurality of machine learning models are output. In some embodiments, outputting trained plurality of machine learning models may comprise: storing one or more of the models in at least one non-transitory computer-readable storage medium (e.g., memory) for subsequent access, providing the model(s) to a recipient (e.g., transmitting data associated with the model(s) to a recipient using any suitable communication network or other means), displaying information associate with the model(s) to a user via a graphical user interface, and/or any other suitable manner of outputting the trained models, as aspects of the technology described herein are not limited in this respect. For example, the trained machine learning models may be stored in a data store, such as the machine learning model data store 454 described herein including at least with respect to FIG. 4.

Training Data Generation

FIG. 7A and FIG. 7B are diagrams depicting an exemplary technique for generating training data comprising simulated expression data, according to some embodiments of the technology described herein.

FIG. 7A is a diagram depicting an exemplary method 700 for training one or more machine learning models, including generating simulated expression data (e.g., to use as training data, as described herein including at least with respect to FIG. 6). In some embodiments, the simulated expression data may be generated by combining samples of expression data from tumor cells (e.g., cancer cells), also referred to herein as “malignant cells”, and tumor microenvironment cells (e.g., immune cells, stromal cells, etc.), as shown in branches 710 and 720 of the method 700. An exemplary process for generating artificial mixes of expression data is described herein below with respect to FIG. 7A.

FIG. 7B is a diagram depicting an example of generating artificial mixes of expression data to imitate real tissue, according to some embodiments of the technology described herein. In some embodiments, the expression data is derived from one or more sorted cell types/subtypes representing one or more biological states (e.g., positive gene regulation, negative gene regulation, etc.), as shown in branch 730. In some embodiments, the one or more cell types/subtypes are mixed in different proportions to generate artificial mixes, as shown in branches 740 and 750.

Data Collection, Analysis, and Preprocessing

According to some embodiments, the expression data may be obtained as described herein including at least with respect to FIG. 1 and the sections “Expression Data” and “Obtaining Expression Data”. For example, a large number of samples of sorted tumor and TME cells may be used to construct the artificial mixes of expression data. In some embodiments, the number of samples may be at least 5,000, at least 10,000, at least 15,000, at least 20,000, at least 30,000, at least 50,000, at least 100,000, or any number of suitable samples. In some embodiments, open-source datasets such as Gene Expression Omnibus (GEO) and ArrayExpress may be used. In some embodiments, the datasets used may be selected so as to satisfy the following criteria: only Homo sapiens, standard RNA-seq (without polyA depletion, targeted panel, etc.) with read length higher 31 bp. In some embodiments, for constructing artificial mixtures, only relevant cell types for the particular disease being analyzed (e.g., particular type of tumor) may be used. In contrast, for the analysis of gene expression specificity data for all cell types may instead be used.

In some embodiments, selection of datasets may be based on both biological and bioinformatic parameters. For example, datasets with samples cultivated in conditions close to normal physiological conditions may be used. In some embodiments, datasets with abnormal stimulation were excluded, like datasets of CD4+ T-cells hyper stimulated with phorbol 12-myristate 13-acetate and ionomycin activation or macrophages co-cultured with an excessive number of bacterial cultures. In some embodiments, only those samples having at least 4 million coding read counts were used.

In some embodiments, quality control may be performed on the expression data prior to construction of the artificial mixes (e.g., to exclude strange or unreliable datasets). For example, if some samples of CD4+ T cells show no or very low expression of CD45, CD4 or CD3 genes, they may be excluded. The same may done for other cell types, in some embodiments. For example, samples for some cell types may be excluded if they significantly express genes that are not typical for that type of cell (e.g., if in a sample of T cells, CD19, CD33, MS4A1, etc. were expressed in significant amounts, while in most other T cell samples these expressions were low). In some embodiments, samples of CD4+ T cells may be removed if they express significant amounts of CD8 genes. In some embodiments, several methods of expression analysis like t-SNE or PCA with different gene sets may be used to visualize the similarities and differences between datasets. If a particular cell type from one dataset fails to cluster with the same cell type in the other datasets (e.g., in a t-SNE, PCA, or other plot), then the one dataset may be further analyzed as part of quality control, and some or all of the data from that dataset may be excluded.

Mixes Construction

According to some embodiments, a variety of artificial mixes of expression data (e.g., representing simulated tumor tissue) may be constructed using samples prepared as described herein above. Artificial mixes may be generated using sample expressions in TPM (transcripts per million) units, such that the gene expressions for an overall sample are formed as a linear combination of the expressions of individual cells from that sample. In some embodiments, expression data from samples of various cell types may be mixed in predetermined proportions. As shown in FIG. 7A, simulated expression data for tumor cells (e.g., generated as shown in branch 710) may be combined with simulated expression data for TME cells (e.g., generated as shown in branch 720).

Referring now to branch 720, an exemplary process for generating simulated TME expression data is shown. In the illustrated example, samples of each cell type (e.g., samples of expression data, such as of genes GSE1, GSE2, GSE3, or GSE4, as shown) may be rebalanced by datasets (e.g., reducing the weight of datasets with a large number of samples) and subtypes (e.g., changing the proportions of subtypes of a sample). Techniques for rebalancing are described herein including with respect to the “Rebalancing by datasets” and “Rebalancing by subtypes” sections. For each cell type, multiple samples may then be randomly selected and averaged. Then, for some or all of the cell types being used, the rebalanced/averaged samples may be mixed together in particular proportions (e.g., so as to simulate a real tumor microenvironment).

Referring now to branch 710, an exemplary process for generating simulated tumor expression data is shown. In the illustrated example, random samples of cancer cells (e.g., NSCLC, ccRCC, Mel, HNCK, etc.) may be selected. Then, hyperexpression noise may be added to the resulting expression data to account for abnormal expression of genes by tumor cells. For example, tumor cells sometimes express genes which are ordinarily absent in the parental cell type. When this is the case for specific, semi-specific, or marker genes that are linked to immune or stromal cells within the TME, the overexpressed genes may interfere with the deconvolution techniques described herein. Regardless of whether hyperexpression noise is included, the result of branch 710 may be simulated tumor expression data.

As shown in FIG. 7A, the simulated expression data for the tumor cells (e.g., generated as shown in branch 710) and the simulated expression data for the TME cells (e.g., generated as shown in branch 720) may be combined into an artificial mix (referred to in FIG. 7A as an “expression mix”). In some embodiments, the simulated expression data for the tumor cells and the simulated expression data for the TME cells may be mixed together in a random proportion based on a given distribution for cancer cells. In some embodiments, noise may then be added to the mix to mimic technical noise and noise resulting from biological variability. Each type of noise may be specified according to one or more suitable distributions. For example, as shown in FIG. 7A, the technical noise may be specified by a Poisson distribution, while the noise resulting from biological variability may be specified according to a normal distribution. However, in some embodiments, technical noise may have multiple components, which may be specified by other distributions. For example, another component of technical noise may be specified by a non-Poisson distribution. Regardless of how the artificial mix is generated, in some embodiments the artificial mix may be representative of an artificial tumor, including the TME.

The inventors have recognized and appreciated that, when creating artificial mixes, it may be desirable to use different cells of the same type from different samples. Using a small number of samples for the mixes, or even just one sample for each cell type, would provide poor performance on real tumor samples (e.g., due to the variability of cell states and their expressions, as well as noise due to limited numbers of read counts for different expressions, alignment errors and other causes of technical noise). Therefore, when creating artificial mixtures, the inventors have recognized that is may be desirable to use as many available cell samples as possible.

Accordingly, for this example, a large number of RNA-seq samples (e.g., at least one hundred, at least five hundred, at least one thousand, at least two thousand, or at least five thousand samples) of various cell types were collected. In some embodiments, a number of datasets of tumor cells (e.g., pure cancer cells for various diagnoses, cancer cell lines or sorted from tumors) may also be collected. For each cell type, there may be a corresponding number of samples from different datasets.

In some embodiments, as described herein including with respect to FIG. 6, the artificial mixes may be used as training datasets for training one or more machine learning models. In some embodiments, the machine learning models may be a gene (e.g., a gene associated with tumor cells). Accordingly, in some embodiments many artificial mixes may be generated to train models for each specific gene.

Averaging of Samples

In some embodiments, multiple samples for each cell type may be averaged in any suitable manner (e.g., to improve the quality of samples before adding artificial noise). For example, in some embodiments, averaging may be performed in groups of two, such that an averaged sample of 4 million reads may contain information on 8 million reads. In some embodiments, averaging across multiple samples may reduce the noise in the expression caused by technical factors during sequencing.

Samples Rebalancing

Since different datasets and cell subtypes can vary significantly in the number of available cell samples, in some embodiments the number of samples may be rebalanced. As described herein below, in one example, the samples may be rebalanced by datasets, then by cell subtypes.

Rebalancing by Datasets

In some embodiments, the number of samples of sorted cells in datasets may range from one to several hundred (e.g., at least five, at least ten, at least 50, or at least 100 samples). Typically, each dataset may contain samples of one or two cell types, sorted and sequenced in the same way. Cell samples within the same dataset may also have specific conditions, such as a specific set of markers for sorting or a specific disease of patients from whom the cells were taken. Datasets with a large number of samples can lead to overtraining of models for such datasets. To reduce the weight of datasets with a large number of samples, samples of all datasets are resampled in order to rebalance by datasets.

For example, in some embodiments, for each dataset the number of samples are resampled with replacement to number N_{dataset,new}.

$${N}_{dataset , new} = {N}_{max} * {( \frac{{N}_{dataset , old}}{{N}_{max}} )}^{1 - rebalance parameter}$$

Where Nₘₐₓ is number of samples in the largest dataset (e.g., for the particular cell type) and N_{dataset,old} is the original number of samples in the dataset. The rebalance parameter in the equation is a value in the range [0, 1], where 0 means there is no change in the number of samples, and 1 means that for each dataset there will be the same number of samples. In some embodiments, the rebalancing parameter may be selected during training.

Rebalancing by Cell Subtypes

For a number of cell types, in addition to samples of this type, there may also be samples of more specific subtypes. The number of available subtype samples may not coincide with those ratios that are specified during the formation of mixes with these subtypes, in some cases. Therefore, when creating mixes for the cell type, samples of its subtypes may be rebalanced.

For example, in some embodiments, there may be significantly more CD4+ T cells (and T helpers with Tregs) samples available than CD8+ T cells. In this case, to form an average T cells sample, proportions of CD4+ and CD8+ T cells samples may be changed before the random selection of samples. For example, the proportions may be chosen similar to the ratios of the predicted average RNA fractions for the TCGA or PBMC samples for these cell types. In some embodiments, the predictions may be obtained using one or more linear models trained on mixes with equal cell proportions.

The subtype rebalancing algorithm may be as follows. To rebalance each subtype for a given type, resample with replacement a number of samples equal to:

$${P}_{subtype} * \frac{msize}{{min}_{p}} + 1$$

Where P_{subtype} is a number reflecting the proportion of a given subtype (e.g., the proportion of this subtype among all subtypes for the given type, which may be represented as the number of samples for the subtype divided by the total number of samples for the type); msize is the maximum number of samples among all the subtypes for the given type, and min_P is the minimum number P_{subtype} between all subtypes. According to some embodiments, the rebalancing operation may be performed recursively for all nested subtypes (e.g., subtypes which themselves have subtypes

TME Cells Proportion Generation

According to some embodiments, the resulting samples of different cell types may be mixed with one another in random ratios in order to generate the simulated TME expression data. For example, a first set of artificial mixes may be generated using random proportions of each cell type:

$${f}_{cell} = \frac{{R}_{cell} {K}_{cell}}{{\sum }_{cell} {R}_{cell} {K}_{cell}}$$

Where R_{cell} is a random number distributed uniformly from 0 to 1 and K_{cell} is the coefficient for the particular cell type.

According to some embodiments, the coefficient K_{cell} in the above equations may be chosen so that the most likely ratios of cells mRNA are close to what is observed in TCGA or PBMC samples. These approximate ratios may be calculated from the TCGA or PBMC samples, using models trained without using such ratios. For example, a vector of numbers may be used, reflecting approximate proportions for a given type of tissue. Each number of the vector is multiplied by a random number from 0 to 1. The resulting coefficients are normalized to the sum and used in a linear combination. In some embodiments, K_{cell} may be selected from Table 5, which specifies, for each of multiple cell types, the most likely proportion of the cell type based on tumor tissue and blood (PBMC).

TABLE 5
This table specifies, for each of multiple cell types, the most likely
proportion of the cell type based on tumor tissue and blood (PBMC).

| Cell type | Solid tumors | PBMC |
|---|---|---|
| B cells | 11 | 20 |
| Plasma B cells | 6 | 3 |
| Non plasm B cells | 5 | 17 |
| T cells | 15 | 100 |
| CD4 T cells | 7 | 50 |
| Tregs | 4 | 2 |
| CD8 T cells | 8 | 50 |
| CD8 T cells PD1 low | 4 | 48 |
| CD8 T cells PD1 high | 4 | 2 |
| NK cells | 2 | 16 |
| Monocytes | 2 | 80 |
| Macrophages | 40 | 1 |
| Neutrophils | 2 | 10 |
| Fibroblasts | 50 | 1 |
| Endothelium | 36 | 1 |
| T helpers | 3 | 48 |
| Macrophages M1 | 12 | 0.5 |
| Macrophages M2 | 28 | 0.5 |

Noise Generation

As shown in FIG. 7A, after the artificial mixes have been generated, noise (e.g., technical noise, uniform noise, or any suitable form of noise) may be added to the expression data. For example, noise may be generated and added to the expression data according to the process described herein below:

Tᵢ^{mix}^{_{after}}=Tᵢ^{mix}^{_{before}}+Noise(Tᵢ^{mix}^{_{before}})

In some embodiments, expression of each gene may contribute noise to the overall tissue expression. For example, the expression of a single gene (Tᵢ^{j}) could be represented as a sum:

Tᵢ^{j}=μ_{T}_{ᵢ}+Pᵢ^{j}+Nₚᵣₑₚ_{ᵢ}+N_{bio}_{ᵢ}

Where u_{T}_{ᵢ} represents the true expression of the gene, Pᵢ^{j} represents Poisson technical noise, Nₚᵣₑₚ_{ᵢ} represents normally distributed noise derived from sequencing library preparation, and N_{bio}_{ᵢ} represents variable biological noise.

In some embodiments, a relative standard deviation of Poisson technical noise (δ_{P}_{ᵢ}) and a relative standard deviation of the normally distributed noise (δ_{N}_{ᵢ}) are used to calculate a quantitative relative standard deviation:

δᵢ=\sqrt{δ_{P}_{ᵢ}²+δ_{N}_{ᵢ}²}

Technical variability may result from differences in sample and library preparation (non-Poisson noise) and random transcript selection on the sequencer track due to limited coverage (Poisson noise). Many cell types of the TME may typically occupy a small fraction in tumor samples. Therefore, the inventors have recognized and appreciated that it may be important to consider different levels of variability or noise for different genes, depending on the level of their expression. For example, in some embodiments, a TPM-based mathematical noise model is provided, which accounts for technical noise (both Poisson and non-Poisson). In some embodiments, this model of variability may be added to the artificial mixes generated to train the machine learning models, as described herein. In some embodiments, technical non-Poisson noise is assumed to be normally distributed. These may account for variability in the library preparation, alignment or variations in human handling of different samples. In contrast, Poisson noise is a type of technical noise which may be associated with the sequencing coverage or number of read counts and may not be normally distributed. The resulting dependence of technical noise on coverage and gene expression could be expressed by a formula:

$${δ}_{{P}_{i}} = α \sqrt{\frac{1}{{ℓ}_{i} {\overline{T}}_{i} R}}$$

Where [symbol] ᵢ is an effective gene length, T̅ᵢ is a mean TPM in technical replicates, R is read counts, and α is an estimated proportional coefficient. According to this equation, the lower the coverage the higher the variability. According to this equation, genes with a low expression will present with a high level of Poisson noise.

In addition to technical noise, biological noise, which may be associated with different activated states of a cell, can contribute to the overall variance in an RNA-seq sample. In some embodiments, there may be no need to add biological noise to artificial mixes, as this noise may already be present through the use of RNA-seq data derived from cell subsets representing a variation of biological states.

In some embodiments, the analysis of noise contribution due to single gene expression, as described herein, may be applied to simulate technical and biological noise in artificial mixes. For example, noise may be added to total gene expression in two summands:

$${T}_{i}^{{mix}_{after}} = {T}_{i}^{{mix}_{before}} + β \sqrt{\frac{{T}_{i}^{{mix}_{before}}}{{l}_{i}}} {ξ}_{P} + γ {T}_{i}^{{mix}_{before}} {ξ}_{N}$$

Where ξ_{P}, ξ_{N}˜N(0,1), β is the coefficient of Poisson noise level coefficient, and γ is the coefficient of uniform level non-Poisson noise.

The noise model described herein may be used to add technical (both Poisson and non-Poisson) variation to artificial mixes. This results in artificial mixes which better mimic real tissues. Improved artificial mixes may subsequently be used to train the deconvolution algorithm (e.g., as described herein including with respect to FIG. 6) to ensure model stability when encountering real sequencing variability.

Additional examples and techniques for generating training data including simulated expression data are described in in the “Cellular Deconvolution” section and in U.S. Patent Publication No. 2021-0287759, entitled “SYSTEMS AND METHODS FOR DECONVOLUTION OF EXPRESSION DATA”, the entire contents of which is herein incorporated by reference in its entirety.

Cellular Deconvolution

FIG. 8A is a flowchart depicting a process 800 for determining an composition percentage for at least one cell type. In some embodiments, the process 800 may be carried out on a computing device (e.g., as described herein including at least with respect to FIG. 24). For example, the computing device may include at least one processor, and at least one non-transitory storage medium storing processor-executable instructions which, when executed, perform the acts of process 800. The process 800 may be carried out, for example, in a clinical setting or a laboratory setting, by one or more computing devices such as by computing device 104.

At act 802, the process 800 begins with obtaining expression data for a biological sample from a subject. In some embodiments, obtaining expression data may include obtaining expression data from a biological sample that has been previously obtained from a subject using any suitable techniques. In some embodiments, obtaining the expression data may include obtaining expression data that has been previously obtained from a biological sample (e.g., obtaining the expression data by accessing a database.) In some embodiments, the expression data is RNA expression data. Examples of RNA expression data are provided herein. In some embodiments, the subject may have, be suspected of having, or be at risk of having cancer. The biological sample may comprise a biopsy (e.g., of a tumor or other diseased tissue of the subject), any of the embodiments described herein including with respect to the “Biological Samples” section, or any other suitable type of biological sample. In some embodiments, the origin or preparation of the expression data may include any of the embodiments described with respect to the “Expression Data” and “Obtaining Expression Data” sections. For example, the expression data may be RNA expression data extracted using any suitable techniques. As another example, the expression data obtained at act 802 may comprise RNA expression data measured in TPM.

In some embodiments, the expression data may be stored on at least one storage medium and accessed as part of act 802. For example, the expression data may be stored in one or more files or in a database, then read. In some embodiments, the at least one storage medium storing the RNA expression data may be local to the computing device (e.g., stored on the same at least one non-transitory storage medium), or may be external to the computing device (e.g., stored in a remote database or a cloud storage environment). The expression data may be stored on a single storage medium or may be distributed across multiple storage mediums.

In some embodiments, the expression data of act 802 may include first expression data associated with a first set of genes associated with a first cell type (e.g., a cell type of the cell types and/or subtypes being analyzed in the biological sample). In some embodiments, the first set of genes may comprise genes that are specific and/or semi-specific to the first cell type. For example, for the endothelium cell type, the set of genes may comprise: ANGPT2, APLN, CDH5, CLEC14A, ECSCR, EMCN, ENG, ESAM, ESM1, FLT1, HHIP, KDR, MMRN1, MMRN2, NOS3, PECAMI, PTPRB, RASIPI, ROBO4, SELE, TEK, TIE1, and/or VWF. In some embodiments, the first set of genes may be the same as a set of genes, or a subset of a set of genes, used as part of training a corresponding non-linear regression model for the cell type.

At act 804, the process 800 proceeds with determining first RNA percentages for at least the first cell type. As shown, determining first RNA percentages for the first cell type may comprise processing first expression data associated with a first set of genes for the first cell type with a first non-linear regression model (e.g., of the one or more non-linear regression models) to determine the first RNA percentages for the first cell type. For example, the first expression data may be provided as input to the first non-linear regression model. In some embodiments, other information may be provided as part of the input to the non-linear regression model. For example, a median of the expression data may be included as part of the input to the non-linear regression model. In some embodiments, any other suitable information may additionally or alternatively be provided as part of the input (e.g., an average of the expression data, a median or average of a subset of the expression data, or any other suitable statistics derived from or otherwise relating to the expression data).

In some embodiments, parts of act 804 may be repeated and/or performed in parallel for each cell type and/or subtype being analyzed. For example, a subset of the expression data may be provided as input to each non-linear regression model for each respective cell type and/or subtype.

In some embodiments, the output of the non-linear regression model may comprise information representing estimated percentages of RNA from the first cell type in the sample.

In some embodiments, process 800 then proceeds to act 806 for outputting the first RNA percentages. Regardless of the architecture or input(s) to the non-linear regression models, including the non-linear regression model for the first cell type, the output(s) of the one or more non-linear regression models may be combined, stored, or otherwise post-processed as part of process 800. For example, the RNA percentages for each cell type may be stored locally on the computing device used to perform process 800 (e.g., on the non-transitory storage medium). In some embodiments, the RNA percentages may be stored in one or more external storage mediums (e.g., such as a remote database or cloud storage environment).

FIG. 8B is an example implementation of process 800 for determining one or more RNA percentages based on expression data. In some embodiments, implementing process 800 may include any suitable combination of acts included in the example flowchart of FIG. 8B. In some embodiments, implementing process 800 may include additional or alternative steps that are not shown in FIG. 8B. For example, executing process 800 may include every act included in the example flowchart. Alternatively, process 800 may include only a subset of the acts included in the example flowchart (e.g., acts 812 and 816, acts 812, 814, 816, and 818, acts 812, 814 and 816, etc.).

In some embodiments, the example implementation 820 begins at act 812, where expression data is obtained for a biological sample from a subject. Obtaining expression data for a biological sample from a subject is described herein above including with respect to act 802 of FIG. 8A.

In some embodiments, act 812 may include obtaining first expression data and second expression data. The first expression data may be associated with a first set of genes that is associated with a first cell type, while the second expression data may be associated with a second set of genes that is associated with a second cell type. For example, the first expression data may be associated with a first set of genes that is associated with B cells, while the second expression data may be associated with a second set of genes that is associated with T cells. Additionally or alternatively, the first expression data may be associated with a first set of genes associated with a first cell subtype, while the second expression data may be associated with a second set of genes associated with a second cell subtype. For example, the first expression data may be associated with a first set of genes associated with CD4+ cells, while the second expression data may be associated with a second set of genes associated with CD8+ cells.

In some embodiments, the example process 820 proceeds to act 814, where the expression data is pre-processed. In some embodiments, the pre-processing may make the expression data suitable to be processed using the one or more non-linear regression models. For example, the expression data may be sorted, combined, organized into batches, filtered, or pre-processed with any other suitable techniques.

After the expression data is pre-processed, example process 820 proceeds to act 816, where a plurality of RNA percentages may be determined for a plurality of cell types using the expression data and one or more non-linear regression models (e.g., at least five, at least ten, at least fifteen, models.)

In some embodiments, a separate non-linear regression model may be used to estimate RNA percentages for each cell type and/or subtype. For example, act 816 may include act 816a and act 816b, each of which includes using a separate non-linear regression model trained for determining RNA percentages for the first and second cell types and/or subtypes, respectively. Act 816a includes determining first RNA percentages for the first cell type using the first expression data and a first non-linear regression model. Act 816b includes determining second RNA percentages for the second cell type using the second expression data and a second non-linear regression model. In some embodiments, act 816 may include only one of acts 816a and 816b. In some embodiments, act 816 may include using one or more additional non-linear regression models for determining RNA percentages for one or more other cell types (e.g., a third cell type or subtype). An example implementation of act 816a is described herein including with respect to FIG. 8C.

In some embodiments, the RNA percentages obtained at act 816 are output at act 818 of process 820.

FIG. 8C shows an example implementation of act 816a for determining, using the first expression data and the first non-linear regression model, first RNA percentages for the first cell type. As shown, in some embodiments, the first non-linear regression model may include a first sub-model and/or a second sub-model for processing the first expression data.

In some embodiments, the first expression data may include first expression data associated with a first set of genes associated with the first cell type, as well as second expression data associated with a second set of genes associated with the first cell type.

In some embodiments, the example implementation begins at act 832, for predicting first values for the estimated percentages of RNA from the first cell type, using a first sub-model. In some embodiments, the first expression data associated with the first set of genes and/or any other input information may be provided as input to the first sub-model of the non-linear regression model, and the output may be one or more predicted percentages of RNA from the first cell type.

In some embodiments, after predicting the first values, the example implementation proceeds to act 834, for predicting second values for the estimated percentage of RNA from the first cell type, using a second sub-model. In some embodiments, the second expression data associated with the second set of genes may be provided as input to the second sub-model of the non-linear expression model in addition to the prediction from the first sub-model and/or any other input information provided at the first sub-model. Additionally or alternatively, the first expression data associated with the first set of genes may be provided as input to the second sub-model. According to some embodiments, predictions from multiple non-linear regression models (e.g., the output of the first sub-model of each non-linear regression model for each cell type) may be provided as input to the second sub-model of the non-linear regression model for the first cell type. Regardless of the input to the second sub-model, the output of the second sub-model of the non-linear regression model may be an estimated percentage of RNA from the first cell type in the sample. The output of the second sub-model may comprise the output of the non-linear regression model for the first cell type, in some embodiments.

In some embodiments, the non-linear regression model may comprise more than two sub-models. For example, the second sub-model may be repeated any number of times, with the predictions from one or more of the prior sub-models being included as input each time.

Example Experiments

Experiments were undertaken to test the performance of the machine learning techniques described herein.

Preparation of Datasets

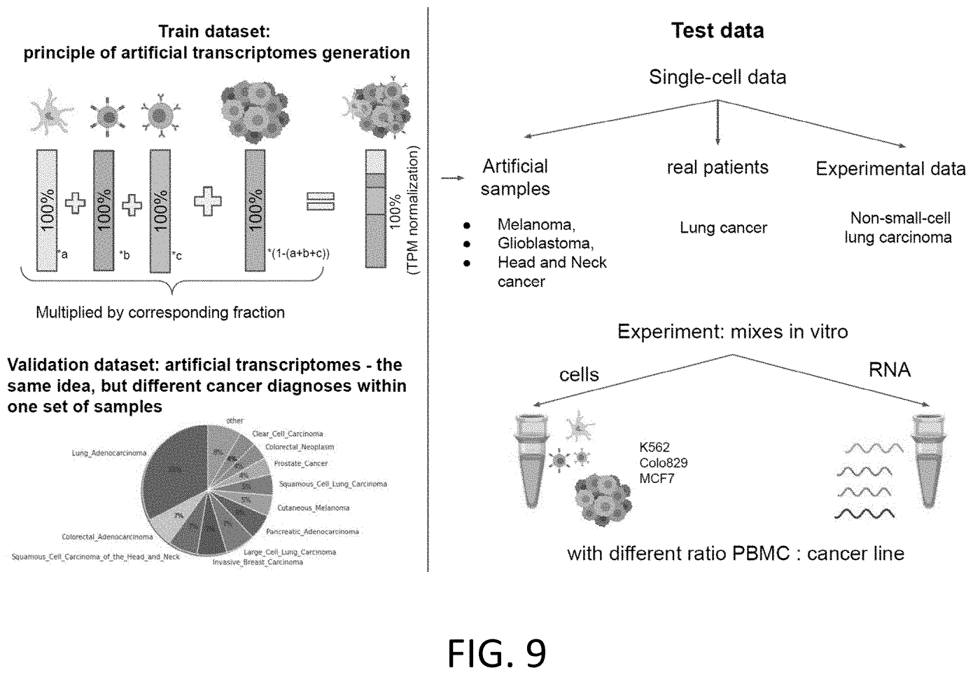

Several types of datasets were used for model development and evaluation. FIG. 9 is a diagram depicting example techniques for preparing data for training, validating, and testing machine learning models for estimating respective TME expression levels of genes in TME cells of one or more biological samples, according to some embodiments of the technology described herein.

First, artificial transcriptomes created from different solid tumor cell lines with the addition of various TME cellular populations (B cells, plasma B cells, CD4+ T cells, CD8+ T cells, macrophages, fibroblasts, endothelium, neutrophils, NK cells, monocytes) were used. Cell proportions were randomly assigned to each TME cell type so that their sum varied from 10% to 60%, while tumor fraction constituted 40-90% of the total sample. Overall, 900000 artificial transcriptomes were generated for training and 100 samples for validation using 7,114 samples of purified TME cell types and 3,143 samples of cancer cell lines.

Single-cell data for different cancer types was used to test the models. For melanoma, glioblastoma and head and neck cancer patient-specific single-cell data scRNAseq-based artificial mixtures were generated following the same strategy described above. Additionally, for lung cancer a public dataset of patient-specific single-cell data without an additional step of artificial transcriptomes generation was used alongside with single-cell data for non-small-cell lung carcinoma.

In vitro experiments were also conducted for additional evaluation of the models, in which different proportions of RNA extracted from PBMCs were mixed with RNA extracted from three cancer cell lines: COL0829 (cutaneous melanoma), MCF-7 (invasive ductal carcinoma), and K562 (chronic myeloid leukemia). The fraction of tumor cell RNA in these in vitro mixtures constituted 25%-95%. After that, gene expression was quantified, and model predictions were compared with the pure cancer cell line expressions.

Model Validation: Validation on Artificial Transcriptomes

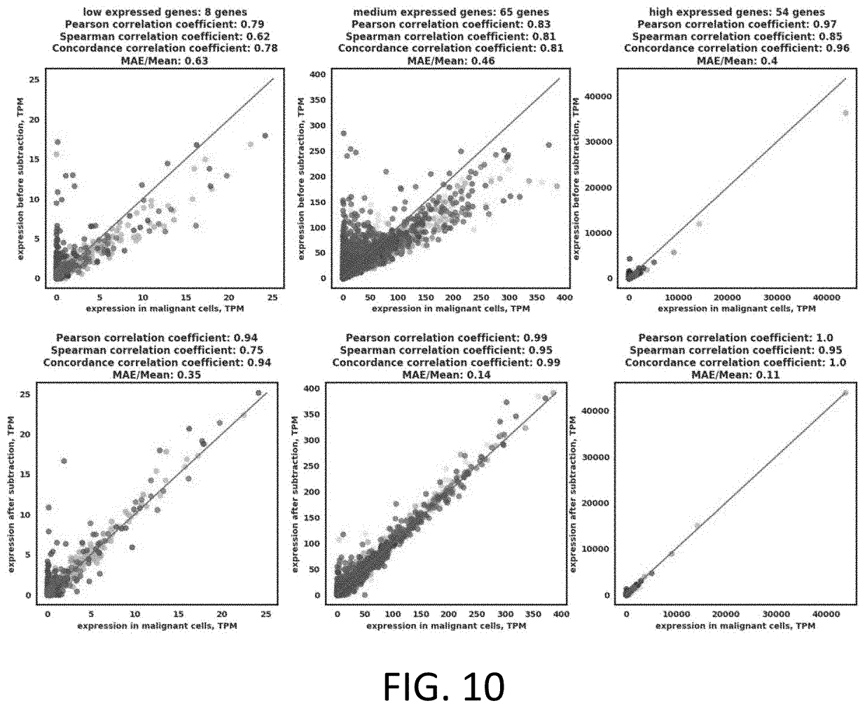

First, the models were validated on the dataset of artificial transcriptomes, in which the percentage of tumor cells varied from 40% to 90%. FIG. 10 demonstrates model performance across all the 127 evaluated genes (e.g., associated with tumor cells) showing that the expression signal obtained using the machine learning techniques described herein significantly improved and became closer to the actual expression of tumor cells. In FIG. 10, the graphs in the top row show the total expression levels of the genes compared to the true tumor expression level those genes. The graphs in the bottom row show the tumor expression levels of the genes, predicted using the machine learning techniques described herein, compared to the true tumor expression level of those genes.

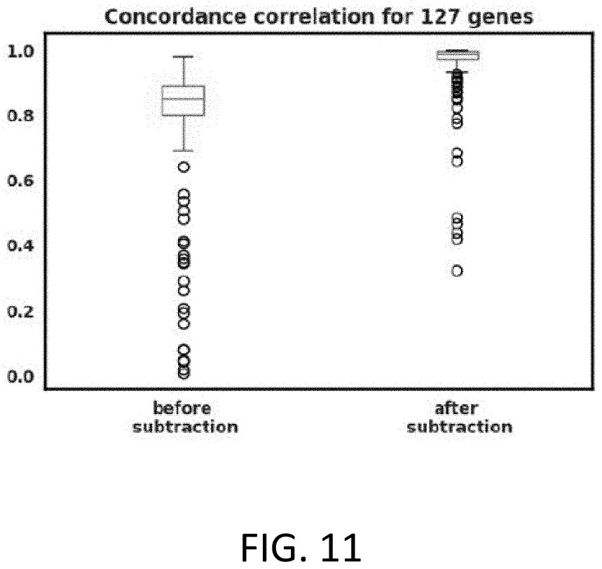

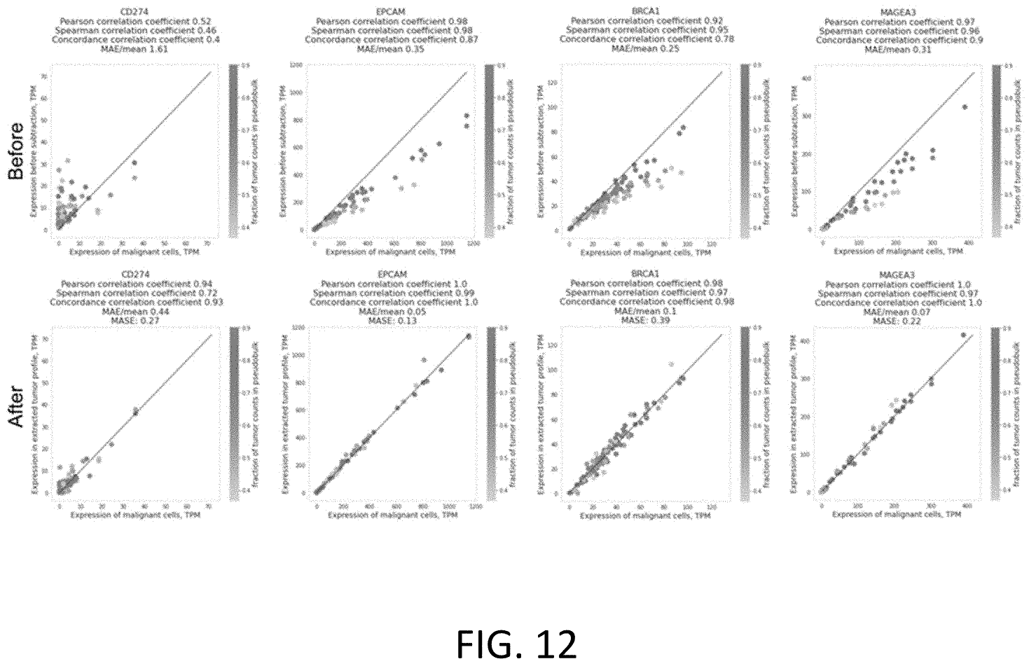

FIG. 11 compares the concordance correlation coefficient for the evaluated gene (a) before using the machine learning techniques described herein (e.g., before subtraction, pure cancer lines) and (b) after using the machine learning techniques described herein (e.g., after subtraction, extracted tumor cell expression). The concordance correlation coefficient between pure cancer cell lines and the extracted tumor cell expression increased on average from 0.85 to 0.98 compared to unprocessed data. Specifically, as shown in FIG. 12, the concordance correlation coefficient increased from 0.4 to 0.93 for CD274, from 0.87 to 1.0 for EPCAM, from 0.78 to 0.98 for BRCA1 and from 0.9 to 1.0 for MAGEA3. FIG. 12 shows examples of the performance of the machine learning techniques on single genes from the artificial transcriptomes dataset.

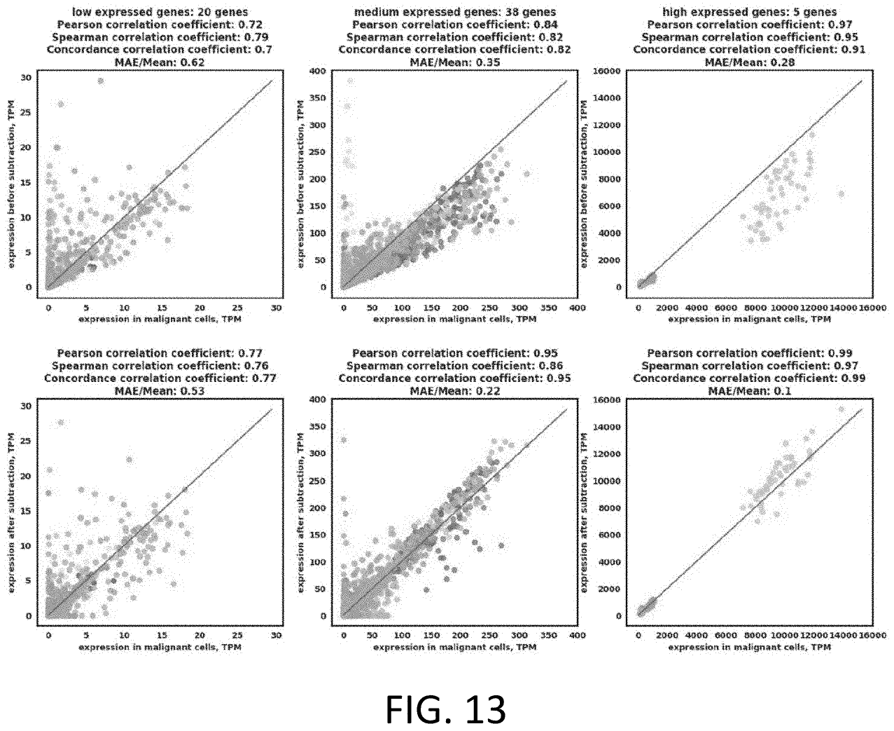

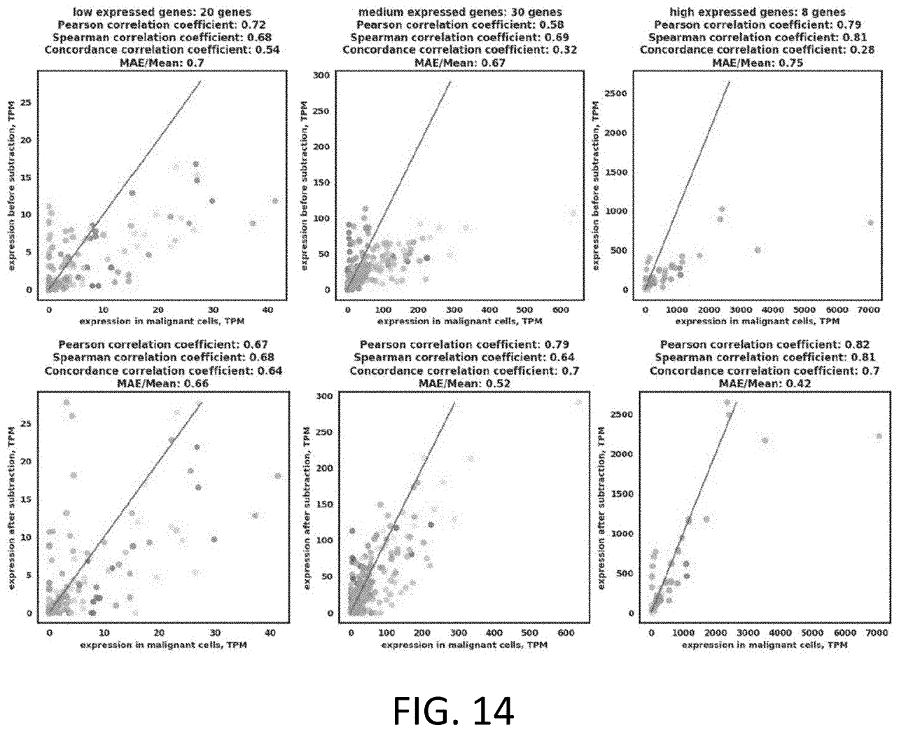

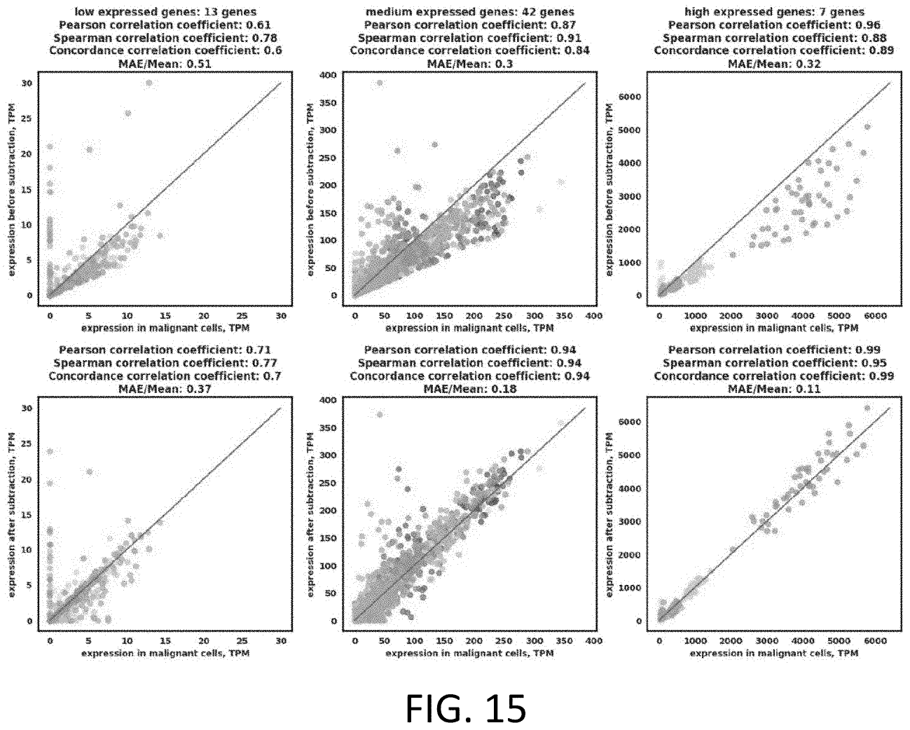

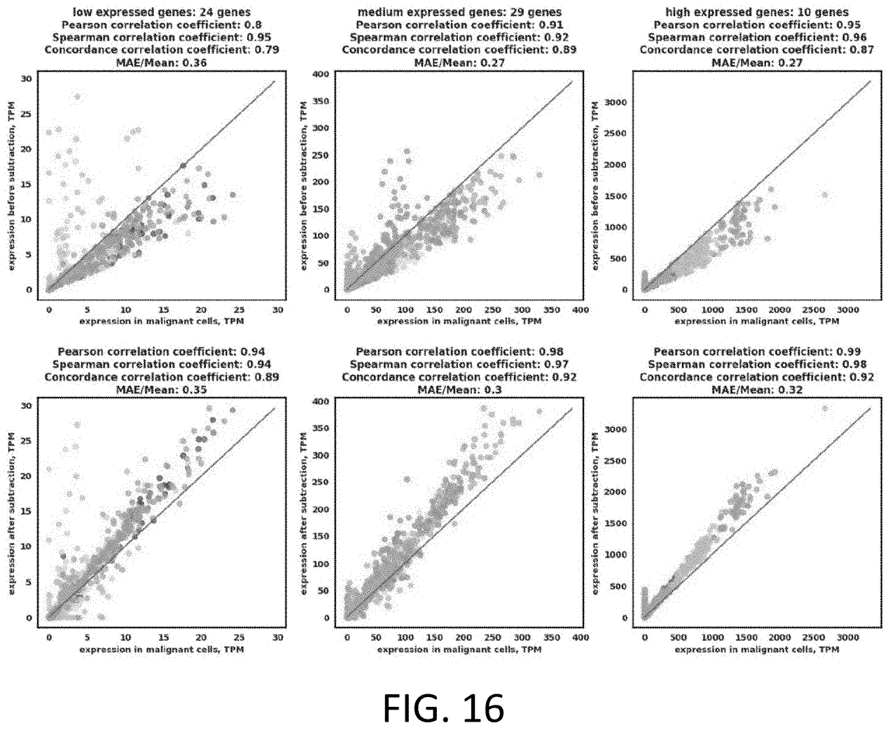

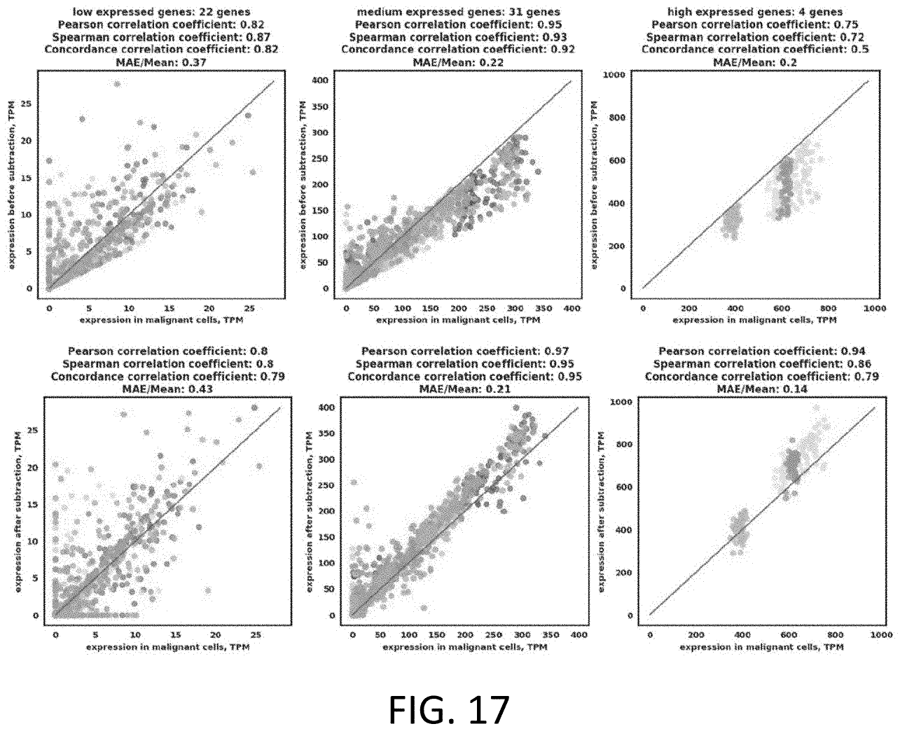

Next, the machine learning techniques were tested on single-cell data from different cancer types. FIG. 13 shows model performance on melanoma single-cell data. FIG. 14 shows model performance on single-cell data for lung cancer. FIG. 15 shows model performance on single-cell data for head and neck cancer. FIG. 16 shows model performance on glioblastoma single cell data. FIG. 17 shows model performance on single-cell data for non-small cell lung carcinoma. In each of FIGS. 13-17, each shade represents one gene, the graphs in the top row show the total expression levels of the genes compared to the true tumor expression level those genes, and the graphs in the bottom row show the tumor expression levels of the genes, predicted using the machine learning techniques described herein, compared to the true tumor expression level of those genes. Concordance correlation values significantly increased for at least 58 genes across all diagnoses after applying the models: from 0.81 to 0.9 in melanoma, from 0.38 to 0.68 in lung cancer, from 0.78 to 0.88 in head and neck cancer, from 0.85 to 0.91 in glioblastoma and from 0.75 to 0.84 in non-small-cell lung carcinoma.

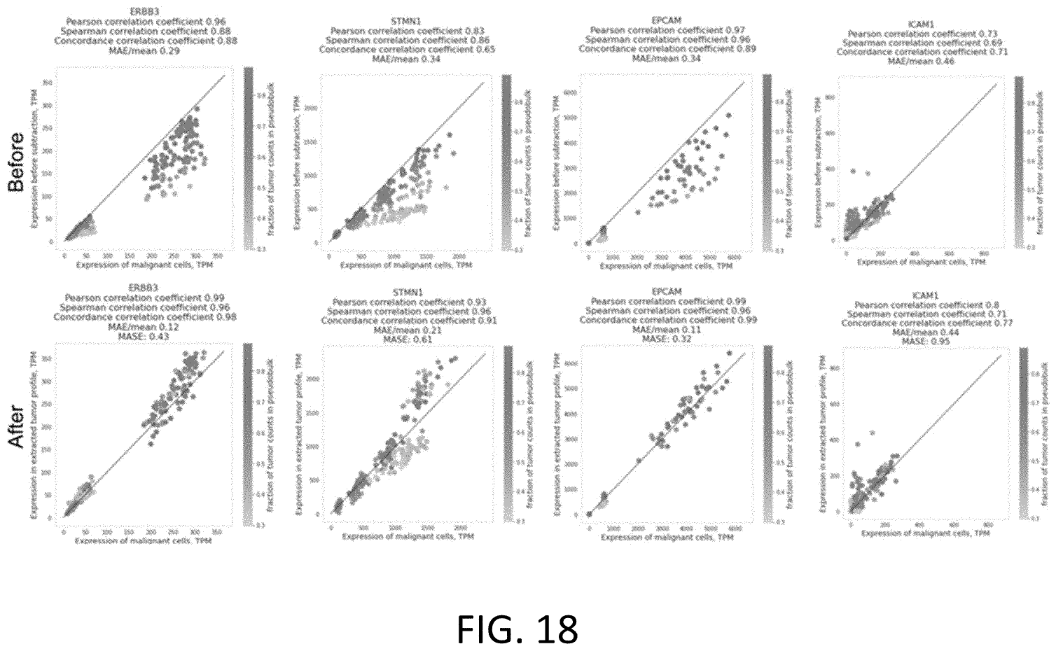

FIG. 18 shows examples of performance of the machine learning techniques on single cells from the scRNA-seq based datasets. In FIG. 18, each data point represents a sample, the graphs in the top row show the total expression levels of the genes compared to the true tumor expression level those genes, and the graphs in the bottom row show the tumor expression levels of the genes, predicted using the machine learning techniques described herein, compared to the true tumor expression level of those genes. In case of single gene examples, concordance correlation values increased by 0.1 for ERBB3 and EPCAM, by 0.26 for STMN1 and by 0.06 for ICAM1.

Model Testing on In Vitro Data

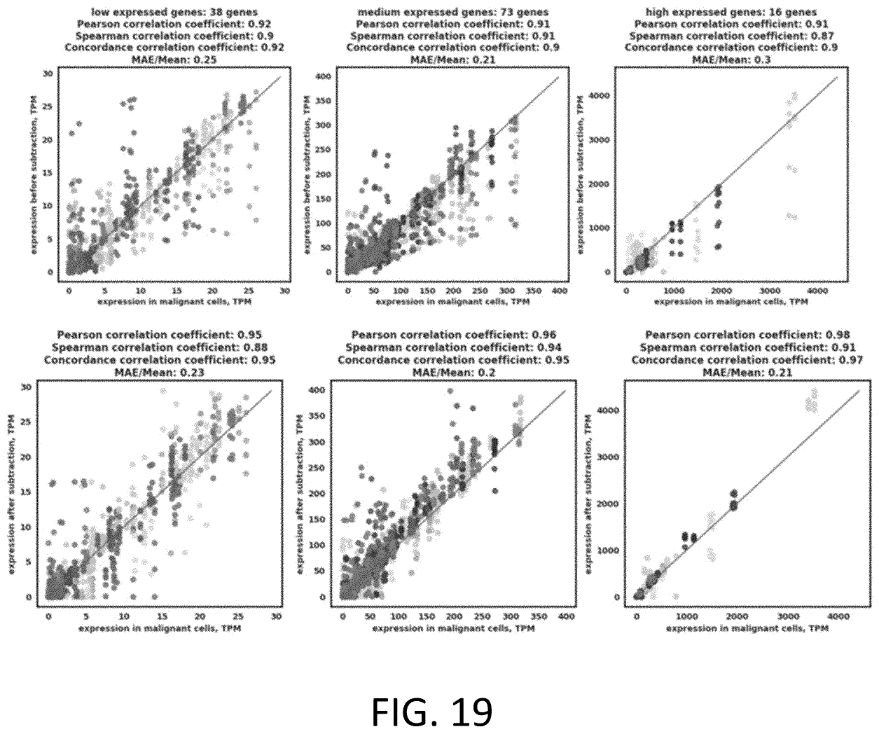

Model evaluation on in vitro data showed that the machine learning techniques described herein improved the concordance correlation coefficient and mean absolute error (MAE) for at least 74 tumor biomarkers (Table 6). Overall, as shown in FIG. 19, concordance correlation values increased from 0.91 to 0.96 in the dataset where RNA fractions were mixed. In FIG. 19, each shade represents one gene, the graphs in the top row show the total expression levels of the genes compared to the true tumor expression level those genes, and the graphs in the bottom row show the tumor expression levels of the genes, predicted using the machine learning techniques described herein, compared to the true tumor expression level of those genes.

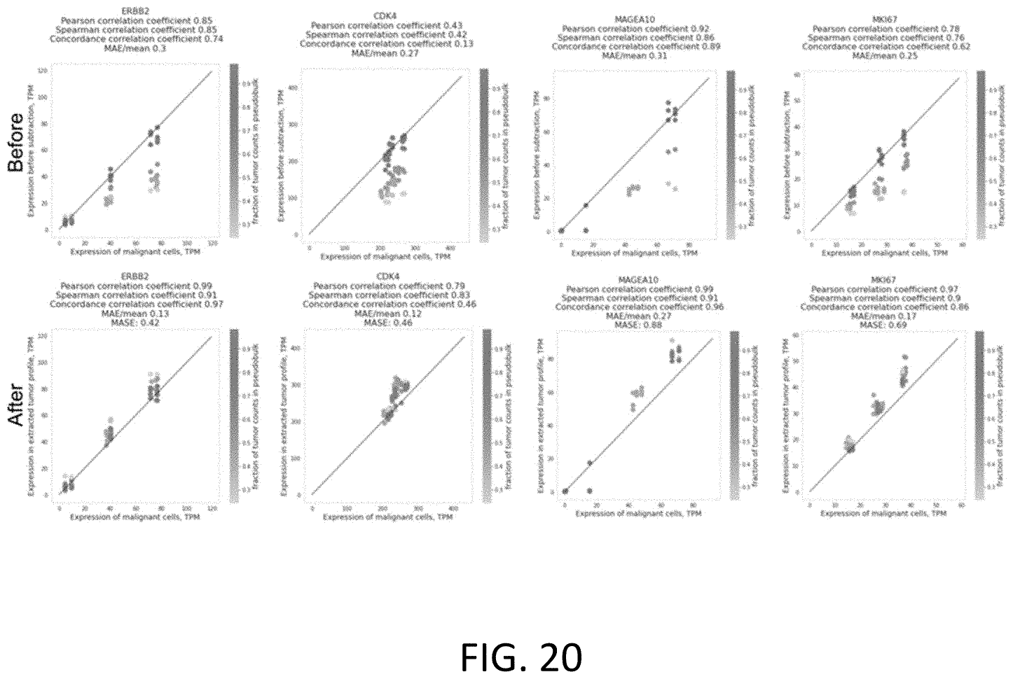

For example, as shown in FIG. 20 the ERBB2 and CDK4 correlation coefficients increased by 0.23 and 0.33, while their MAE were reduced 2-fold. For MAGEA10 and MKI67 genes, concordance correlation coefficients increased from 0.89 to 0.96 and from 0.62 to 0.86, respectively. In FIG. 20, each data point represents a sample, the graphs in the top row show the total expression levels of the genes compared to the true tumor expression level those genes, and the graphs in the bottom row show the tumor expression levels of the genes, predicted using the machine learning techniques described herein, compared to the true tumor expression level of those genes.

TABLE 6
Test-data results for genes in the dataset of in vitro mixed
RNA fractions.

| | | | | MAE/ | Δ |
|---|---|---|---|---|---|
| | Concordance | Pearson | Spearman | Mean | Concordance |
| Gene | (after) | (after) | (after) | (after) | (after-before) |
| BCL2L1 | 0.81 | 0.96 | 0.85 | 0.2 | 0.11 |
| RRM2 | 0.81 | 0.94 | 0.92 | 0.21 | 0.1 |
| IGF2R | 0.84 | 0.92 | 0.79 | 0.31 | 0.13 |
| HDAC2 | 0.84 | 0.95 | 0.91 | 0.19 | 0.03 |
| BCL2L2 | 0.84 | 0.93 | 0.77 | 0.2 | 0.14 |
| CA9 | 0.84 | 0.86 | 0.86 | 0.3 | 0.21 |
| TP53 | 0.85 | 0.94 | 0.94 | 0.31 | 0.02 |
| AURKA | 0.86 | 0.94 | 0.5 | 0.14 | 0.47 |
| MKI67 | 0.86 | 0.97 | 0.9 | 0.17 | 0.24 |
| FGFR4 | 0.86 | 0.93 | 0.9 | 0.18 | 0.25 |
| EGF | 0.87 | 0.97 | 0.49 | 0.35 | 0.06 |
| CD22 | 0.88 | 0.94 | 0.71 | 0.46 | 0.13 |
| FLNA | 0.88 | 0.92 | 0.83 | 0.15 | 0.15 |
| BIRC5 | 0.89 | 0.97 | 0.91 | 0.17 | 0.22 |
| CCNE1 | 0.89 | 0.98 | 0.93 | 0.25 | 0.04 |
| NF1 | 0.9 | 0.97 | 0.91 | 0.16 | 0.04 |
| HDAC9 | 0.9 | 0.9 | 0.69 | 0.43 | 0.49 |
| NF2 | 0.9 | 0.93 | 0.78 | 0.16 | 0.26 |
| AURKB | 0.91 | 0.96 | 0.9 | 0.15 | 0.31 |
| PLK1 | 0.91 | 0.98 | 0.94 | 0.19 | 0.2 |
| CHEK2 | 0.92 | 0.96 | 0.92 | 0.16 | 0.26 |
| TERT | 0.92 | 0.94 | 0.72 | 0.31 | 0.07 |
| STMN1 | 0.92 | 0.98 | 0.93 | 0.19 | 0.1 |
| NAE1 | 0.92 | 0.97 | 0.92 | 0.23 | 0.01 |
| PDGFA | 0.92 | 0.93 | 0.76 | 0.17 | 0.28 |
| RRM1 | 0.92 | 0.99 | 0.81 | 0.18 | 0.12 |
| EPHA2 | 0.93 | 0.97 | 0.86 | 0.21 | 0.18 |
| HDAC1 | 0.93 | 0.98 | 0.86 | 0.14 | 0.02 |
| MAGEA2 | 0.93 | 0.96 | 0.84 | 0.21 | 0.14 |
| MAGEA12 | 0.93 | 0.99 | 0.82 | 0.23 | 0.12 |
| CDKN2A | 0.93 | 0.95 | 0.71 | 0.28 | 0.16 |
| BRCA1 | 0.94 | 0.98 | 0.85 | 0.18 | 0.08 |
| FGFR2 | 0.94 | 0.96 | 0.56 | 0.37 | 0.08 |
| FGFR3 | 0.94 | 0.99 | 0.89 | 0.28 | 0.04 |
| PTK7 | 0.94 | 0.95 | 0.86 | 0.18 | 0.31 |
| MYB | 0.94 | 0.98 | 0.92 | 0.2 | 0.09 |
| MAGEA3 | 0.94 | 0.99 | 0.91 | 0.22 | 0.15 |
| TYMS | 0.94 | 0.97 | 0.89 | 0.2 | 0.14 |
| DLL3 | 0.95 | 0.95 | 0.94 | 0.2 | 0.26 |
| ERBB3 | 0.95 | 0.99 | 0.9 | 0.25 | 0.06 |
| IGF1 | 0.95 | 0.95 | 0.79 | 0.26 | 0.05 |
| IGF1R | 0.95 | 0.98 | 0.89 | 0.21 | 0.1 |
| ADORA2B | 0.95 | 0.96 | 0.66 | 0.25 | 0.13 |
| TUBB3 | 0.95 | 0.98 | 0.83 | 0.17 | 0.17 |
| SMO | 0.95 | 0.99 | 0.75 | 0.28 | 0.1 |
| MAGEA1 | 0.95 | 0.99 | 0.93 | 0.23 | 0.14 |
| ROR2 | 0.95 | 0.99 | 0.91 | 0.27 | 0.05 |
| MAGEA4 | 0.95 | 0.99 | 0.95 | 0.28 | 0.11 |
| CDK2 | 0.95 | 0.99 | 0.93 | 0.2 | 0.12 |
| WT1 | 0.95 | 0.98 | 0.72 | 0.24 | 0.06 |
| ALK | 0.95 | 0.97 | 0.82 | 0.3 | 0.04 |
| MAGEA10 | 0.96 | 0.99 | 0.91 | 0.27 | 0.07 |
| CCND1 | 0.96 | 0.98 | 0.9 | 0.15 | 0.29 |
| PMEL | 0.96 | 0.99 | 0.68 | 0.28 | 0.05 |
| TXNRD1 | 0.96 | 0.98 | 0.93 | 0.13 | 0.3 |
| NOTCH3 | 0.96 | 0.99 | 0.9 | 0.19 | 0.12 |
| ERBB4 | 0.97 | 0.98 | 0.92 | 0.2 | 0.09 |
| NRAS | 0.97 | 0.98 | 0.95 | 0.13 | 0.12 |
| CDKN1A | 0.97 | 0.98 | 0.97 | 0.15 | 0.17 |
| FN1 | 0.97 | 0.99 | 0.78 | 0.22 | 0.18 |
| FLT1 | 0.97 | 0.99 | 0.64 | 0.22 | 0.05 |
| ERBB2 | 0.97 | 0.99 | 0.91 | 0.13 | 0.24 |
| MMP2 | 0.97 | 0.99 | 0.86 | 0.21 | 0.07 |
| EPCAM | 0.97 | 0.99 | 0.92 | 0.14 | 0.16 |
| PGR | 0.98 | 0.99 | 0.91 | 0.15 | 0.18 |
| EGFR | 0.98 | 0.99 | 0.8 | 0.15 | 0.13 |
| ITGB4 | 0.98 | 1 | 0.72 | 0.15 | 0.15 |
| CDH1 | 0.99 | 1 | 0.82 | 0.13 | 0.13 |
| MUC1 | 0.99 | 1 | 0.91 | 0.13 | 0.17 |
| TPBG | 0.99 | 0.99 | 0.82 | 0.09 | 0.16 |
| TACSTD2 | 0.99 | 1 | 0.7 | 0.1 | 0.16 |
| AREG | 0.99 | 0.99 | 0.85 | 0.1 | 0.18 |
| CEACAM6 | 0.99 | 1 | 0.67 | 0.09 | 0.15 |
| SLC39A6 | 0.99 | 1 | 0.9 | 0.09 | 0.17 |

Example Model Parameters

Each machine learning model trained and validated in the above-described experiments comprises a gradient boosted machine learning model trained using the LightGBM, gradient boosting framework.

Table 7 lists example parameters for such a machine learning model:

TABLE 7
Example machine learning model parameters.

| Parameter: | Description | Value: |
|---|---|---|
| subsample | Subsample ratio of the training | 0.9607 |
| | instance. | |
| subsample_freq | Frequency of subsample. | 9.0000 |
| colsample_bytree | Subsample ratio of columns when | 0.2933 |
| | constructing each tree. | |
| reg_alpha | L1 regularization term on weights. | 3.9006 |
| reg_lambda | L2 regularization term on weights. | 2.9380 |
| learning_rate | Boosting learning rate. | 0.0500 |
| max_depth | Maximum tree depth for base learners. | 11.0000 |
| min_child_samples | Minimum number of data needed in a | 271.0000 |
| | child. | |
| num_leaves | Maximum tree leaves for base learners. | 9419.0000 |
| n_estimators | Number of boosted trees to fit. | 3000.0000 |
| n_jobs | Number of parallel threads to use for | 5.0000 |
| | training. | |

### Illustrative Examples

Tumor-specific gene expression analysis plays a decisive role in a wide range of biomedical issues, including, for example, adjustment of personalized genetic-based treatment strategies, determination of prognosis, assessing clinical trial endpoints, identifying new biomarkers, and correcting therapy indications for previously-known biomarkers.

In some embodiments, the effectiveness of a targeted anti-tumor therapy (e.g., monoclonal antibody therapy and CAR-T) depends on the relative abundance of the therapeutic target in tumor cells. As an example, HERCEPTIN® (trastuzumab) is approved by FDA to treat certain breast and stomach cancers but only in patients whose tumors overexpress HER2 (the product of ERBB2 gene), thereby reaffirming the need for accurate determination of intra-tumoral ERBB2 expression. Correct tumor expression determination by the machine learning techniques described herein may allow for avoiding TME-caused false-positive results and the following false-positive indications for HERCEPTIN® (trastuzumab).

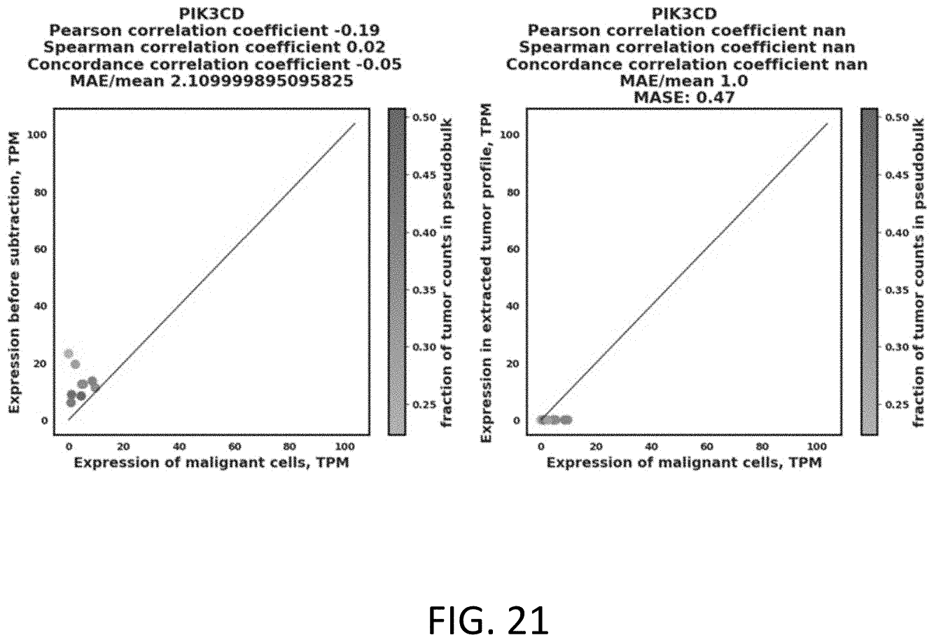

An additional example that demonstrates the range of such false-positive errors is shown for PIK3CD, a target for Idelalisib—FDA approved PI3K selective inhibitor. FIG. 21 shows performance of the machine learning techniques for the PIK3CD gene from the scRNA-seq based datasets. The graph on the left shows the total expression levels of the PI3K gene compared to the true tumor expression level, while the graph on the right shows the tumor expression level of the PI3K gene, predicted using the machine learning techniques described herein, compared to the true tumor expression level of those genes. Each data point represents a different sample.

Despite the moderate initial expression values, the expression of PIK3CD after the application of the machine learning techniques, described herein, is barely detectable, leading to a lack of indications for the use of PIK3CD-specific therapeutics. In the same way, the techniques described herein can be used to correct therapeutic recommendations for the medications targeting any of the genes from Table 6.

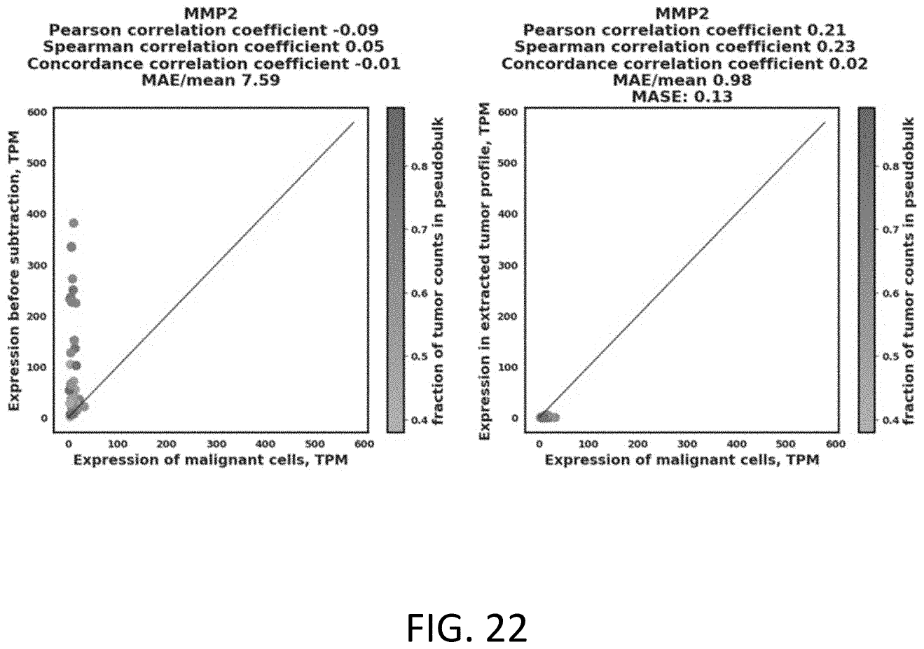

An even more pronounced effect of using the developed algorithm can be observed in the example for MMP2 (matrix metalloproteinase-2), an enzyme that in humans is encoded by the MMP2 gene. FIG. 22 shows performance of the machine learning techniques for the MMP2 gene from the scRNA-seq based datasets. The graph on the left shows the total expression levels of the MMP2 gene compared to the true tumor expression level, while the graph on the right shows the tumor expression level of the MMP2 gene, predicted using the machine learning techniques described herein, compared to the true tumor expression level of those genes. Each data point represents a different sample.

The high level of MMP2 was shown to be associated with both improved disease-free survival and overall survival in breast cancer patients receiving bevacizumab- and trastuzumab-based neoadjuvant chemotherapy. The dramatic change of the gene expression level would entail revising the prognosis for the sample/patient. In the same way, the machine learning techniques described herein can be used to correct prognostic assessments for any of the prognostic/predictive biomarkers listed in Table 6.

Biological Samples

Any of the methods, systems, or other claimed elements may use or be used to analyze a biological sample from a subject. In some embodiments, a biological sample is obtained from a subject having, suspected of having cancer, or at risk of having cancer. The biological sample may be any type of biological sample including, for example, a biological sample of a bodily fluid (e.g., blood, urine or cerebrospinal fluid), one or more cells (e.g., from a scraping or brushing such as a cheek swab or tracheal brushing), a piece of tissue (cheek tissue, muscle tissue, lung tissue, heart tissue, brain tissue, or skin tissue), or some or all of an organ (e.g., brain, lung, liver, bladder, kidney, pancreas, intestines, or muscle), or other types of biological samples (e.g., feces or hair).

In some embodiments, the biological sample is a sample of a tumor from a subject. In some embodiments, the biological sample is a sample of blood from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells. In some embodiments, the sample of tumor can include a mixture of cancerous, non-cancerous, and/or precancerous cells.

Examples of tumors include, but are not limited to, adenomas, fibromas, hemangiomas, lipomas, cervical dysplasia, metaplasia of the lung, leukoplakia, carcinoma, sarcoma, germ cell tumors, melanomas, mesotheliomas, gliomas, and blastoma.

A sample of blood, in some embodiments, refers to a sample comprising cells, e.g., cells from a blood sample. In some embodiments, the sample of blood comprises non-cancerous cells. In some embodiments, the sample of blood comprises precancerous cells. In some embodiments, the sample of blood comprises cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. In some embodiments, a sample of blood is collected to obtain the cell-free nucleic acid (e.g., cell-free DNA) in the blood.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

A sample of tissue, in some embodiments, refers to a sample comprising cells from a tissue. In some embodiments, the sample of the tumor comprises non-cancerous cells from a tissue. In some embodiments, the sample of the tumor comprises precancerous cells from a tissue. In some embodiments, the sample of the tumor comprises cancerous tissue. In some embodiments, the sample can comprise cancerous, precancerous, or non-cancerous cells.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue, or it may be diseased tissue or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

Any of the biological samples described herein may be obtained from the subject using any known technique. See, for example, the following publications on collecting, processing, and storing biological samples, each of which are incorporated herein in its entirety: Biospecimens and biorepositories: from afterthought to science by Vaught et al. (Cancer Epidemiol Biomarkers Prev. 2012 February; 21(2):253-5), and Biological sample collection, processing, storage and information management by Vaught and Henderson (IARC Sci Publ. 2011; (163):23-42).

In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one or more than one cell (a cell biological sample) may be obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixatuve. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example paraffin wax. In some embodiments, the biological sample is a formalin-fixed paraffin-embedded (FFPE) sample. Methods of preparing FFPE samples are known, for example as described by Li et al. JCO Precis Oncol. 2018; 2: PO.17.00091.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris-Cl; 0.5 mM EDTA, pH 9.0)) and other coagulants, and Acids Citrate Dextronse (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −80° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

Methods of the present disclosure encompass obtaining one or more biological samples from a subject for analysis. In some embodiments, one biological sample is collected from a subject for analysis. In some embodiments, more than one (e.g., 2, 3, 4, 5, 6, 7, 8, 9, 10, 11, 12, 13, 14, 15, 16, 17, 18, 19, 20, or more) biological samples are collected from a subject for analysis. In some embodiments, one biological sample from a subject will be analyzed. In some embodiments, more than one (e.g., 2, 3, 4, 5, 6, 7, 8, 9, 10, 11, 12, 13, 14, 15, 16, 17, 18, 19, 20, or more) biological samples may be analyzed. If more than one biological sample from a subject is analyzed, the biological samples may be procured at the same time (e.g., more than one biological sample may be taken in the same procedure), or the biological samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure).

A second or subsequent biological sample may be taken or obtained from the same region (e.g., from the same tumor or area of tissue) or a different region (including, e.g., a different tumor). A second or subsequent biological sample may be taken or obtained from the subject after one or more treatments and may be taken from the same region or a different region. As a non-limiting example, the second or subsequent biological sample may be useful in determining whether the cancer in each biological sample has different characteristics (e.g., in the case of biological samples taken from two physically separate tumors in a patient) or whether the cancer has responded to one or more treatments (e.g., in the case of two or more biological samples from the same tumor or different tumors prior to and subsequent to a treatment). In some embodiments, each of the at least one biological sample is a bodily fluid sample, a cell sample, or a tissue biopsy sample.

In some embodiments, one or more biological specimens are combined (e.g., placed in the same container for preservation) before further processing. For example, a first sample of a first tumor obtained from a subject may be combined with a second sample of a second tumor from the subject, wherein the first and second tumors may or may not be the same tumor. In some embodiments, a first tumor and a second tumor are similar but not the same (e.g., two tumors in the brain of a subject). In some embodiments, a first biological sample and a second biological sample from a subject are sample of different types of tumors (e.g., a tumor in muscle tissue and brain tissue).

In some embodiments, a sample from which RNA and/or DNA is extracted (e.g., a sample of tumor, or a blood sample) is sufficiently large such that at least 2 μg (e.g., at least 2 μg, at least 2.5 μg, at least 3 μg, at least 3.5 μg or more) of RNA can be extracted from it. In some embodiments, the sample from which RNA and/or DNA is extracted can be peripheral blood mononuclear cells (PBMCs). In some embodiments, the sample from which RNA and/or DNA is extracted can be any type of cell suspension. In some embodiments, a sample from which RNA and/or DNA is extracted (e.g., a sample of tumor, or a blood sample) is sufficiently large such that at least 1.8 μg RNA can be extracted from it. In some embodiments, at least 50 mg (e.g., at least 1 mg, at least 2 mg, at least 3 mg, at least 4 mg, at least 5 mg, at least 10 mg, at least 12 mg, at least 15 mg, at least 18 mg, at least 20 mg, at least 22 mg, at least 25 mg, at least 30 mg, at least 35 mg, at least 40 mg, at least 45 mg, or at least 50 mg) of tissue sample is collected from which RNA and/or DNA is extracted. In some embodiments, at least 20 mg of tissue sample is collected from which RNA and/or DNA is extracted. In some embodiments, at least 30 mg of tissue sample is collected. In some embodiments, at least 10-50 mg (e.g., 10-50 mg, 10-15 mg, 10-30 mg, 10-40 mg, 20-30 mg, 20-40 mg, 20-50 mg, or 30-50 mg) of tissue sample is collected from which RNA and/or DNA is extracted. In some embodiments, at least 30 mg of tissue sample is collected. In some embodiments, at least 20-30 mg of tissue sample is collected from which RNA and/or DNA is extracted. In some embodiments, a sample from which RNA and/or DNA is extracted (e.g., a sample of tumor, or a blood sample) is sufficiently large such that at least 0.2 μg (e.g., at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of RNA can be extracted from it. In some embodiments, a sample from which RNA and/or DNA is extracted (e.g., a sample of tumor, or a blood sample) is sufficiently large such that at least 0.1 μg (e.g., at least 100 ng, at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of RNA can be extracted from it.

Subjects

Aspects of this disclosure relate to a biological sample that has been obtained from a subject. In some embodiments, a subject is a mammal (e.g., a human, a mouse, a cat, a dog, a horse, a hamster, a cow, a pig, or other domesticated animal). In some embodiments, a subject is a human. In some embodiments, a subject is an adult human (e.g., of 18 years of age or older). In some embodiments, a subject is a child (e.g., less than 18 years of age). In some embodiments, a human subject is one who has or has been diagnosed with at least one form of cancer.

In some embodiments, a cancer from which a subject suffers is a carcinoma, a sarcoma, a myeloma, a leukemia, a lymphoma, a melanoma, a mesothelioma, a glioma, or a mixed type of cancer that comprises more than one of a carcinoma, a sarcoma, a myeloma, a leukemia, and a lymphoma. Carcinoma refers to a malignant neoplasm of epithelial origin or cancer of the internal or external lining of the body. Sarcoma refers to cancer that originates in supportive and connective tissues such as bones, tendons, cartilage, muscle, and fat. Myeloma is cancer that originates in the plasma cells of bone marrow. Leukemias (“liquid cancers” or “blood cancers”) are cancers of the bone marrow (the site of blood cell production). Lymphomas develop in the glands or nodes of the lymphatic system, a network of vessels, nodes, and organs (specifically the spleen, tonsils, and thymus) that purify bodily fluids and produce infection-fighting white blood cells, or lymphocytes. Melanoma is a type of skin cancer that originates in the melanocytes of the skin. Mesothelioma's cancers arise from the mesothelium, which forms the lining of organs and cavities, such as, for example, the lungs and the abdomen. Glioma develops in the brain, and specifically in the glial cells, which provide physical and metabolic support to neurons. Non-limiting examples of a mixed type of cancer include adenosquamous carcinoma, mixed mesodermal tumor, carcinosarcoma, and teratocarcinoma. In some embodiments, a subject has a tumor. A tumor may be benign or malignant.

In some embodiments, a cancer is any one of the following: skin cancer, lung cancer, breast cancer, prostate cancer, colon cancer, pancreatic cancer, rectal cancer, cervical cancer, and cancer of the uterus. In some embodiments, a subject is at risk for developing cancer, e.g., because the subject has one or more genetic risk factors, or has been exposed to or is being exposed to one or more carcinogens (e.g., cigarette smoke, or chewing tobacco).

Expression Data

Expression data (e.g., indicating expression levels) for a plurality of genes may be used for any of the methods or compositions described herein. The number of genes which may be examined may be up to and inclusive of all the genes of the subject. In some embodiments, expression levels may be examined for all of the genes of a subject. As a non-limiting example, four or more, five or more, six or more, seven or more, eight or more, nine or more, ten or more, eleven or more, twelve or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 35 or more, 40 or more, 50 or more, 60 or more, 70 or more, 80 or more, 90 or more, 100 or more, 125 or more, 150 or more, 175 or more, 200 or more, 225 or more, 250 or more, 275 or more, or 300 or more genes may be used for any evaluation described herein. As another set of non-limiting examples, the expression data may include expression data for at least 5, at least 10, at least 20, at least 25, at least 35, at least 50, at least 75, at least 100, at least 125, at least 150 or more genes selected from the genes listed in Table 1. Additionally or alternatively, the expression data my include expression data for at least 5, at least 10, at least 20, at least 25, at least 35, at least 50, at least 75, at least 100, at least 125, at least 150, at least 200, at least 250, at least 300, at least 350, at least 400 or more genes selected from the genes listed in Table 2.

Any method may be used on a sample from a subject in order to acquire expression data (e.g., indicating expression levels) for the plurality of genes. As a set of non-limiting examples, the expression data may be RNA expression data, DNA expression data, or protein expression data.

DNA expression data, in some embodiments, refers to a level of DNA (e.g., copy number of a chromosome, gene, or other genomic region) in a sample from a subject. The level of DNA in a sample from a subject having cancer may be elevated compared to the level of DNA in a sample from a subject not having cancer, e.g., a gene duplication in a cancer patient's sample. The level of DNA in a sample from a subject having cancer may be reduced and compared to the level of DNA in a sample from a subject not having cancer, e.g., a gene deletion in a cancer patient's sample.

DNA expression data, in some embodiments, refers to data (e.g., sequencing data) for DNA (e.g., coding or non-coding genomic DNA) present in a sample, for example, sequencing data for a gene that is present in a patient's sample. DNA that is present in a sample may or may not be transcribed, but it may be sequenced using DNA sequencing platforms. Such data may be useful, in some embodiments, to determine whether the patient has one or more mutations associated with a particular cancer.

RNA expression data may be acquired using any method known in the art including, but not limited to: whole transcriptome sequencing, total RNA sequencing, mRNA sequencing, targeted RNA sequencing, small RNA sequencing, ribosome profiling, RNA exome capture sequencing, and/or deep RNA sequencing. DNA expression data may be acquired using any method known in the art including any known method of DNA sequencing. For example, DNA sequencing may be used to identify one or more mutations in the DNA of a subject. Any technique used in the art to sequence DNA may be used with the methods and compositions described herein. As a set of non-limiting examples, the DNA may be sequenced through single-molecule real-time sequencing, ion torrent sequencing, pyrosequencing, sequencing by synthesis, sequencing by ligation (SOLiD sequencing), nanopore sequencing, or Sanger sequencing (chain termination sequencing). Protein expression data may be acquired using any method known in the art including, but not limited to: N-terminal amino acid analysis, C-terminal amino acid analysis, Edman degradation (including though use of a machine such as a protein sequenator), or mass spectrometry.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, the expression data comprises whole exome sequencing (WES) data. In some embodiments, the expression data comprises whole genome sequencing (WGS) data. In some embodiments, the expression data comprises next-generation sequencing (NGS) data. In some embodiments, the expression data comprises microarray data.

Obtaining Expression Data

In some embodiments, a method to process expression data (e.g., data obtained from sequencing comprises obtaining expression data for a subject (e.g., a subject who has or has been diagnosed with a cancer). In some embodiments, obtaining expression data comprises obtaining a biological sample and processing it to perform sequencing using any one of the sequencing methods described herein. In some embodiments, expression data is obtained from a lab or center that has performed experiments to obtain expression data (e.g., a lab or center that has performed sequencing). In some embodiments, a lab or center is a medical lab or center.

In some embodiments, expression data is obtained by obtaining a computer storage medium (e.g., a data storage drive) on which the data exists. In some embodiments, expression data is obtained via a secured server (e.g., a SFTP server, or Illumina BaseSpace). In some embodiments, data is obtained in the form of a text-based filed (e.g., a FASTQ file). In some embodiments, a file in which sequencing data is stored also contains quality scores of the sequencing data). In some embodiments, a file in which sequencing data is stored also contains sequence identifier information.

Expression Levels

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

FIG. 23 shows an exemplary process 2300 for processing sequencing data to obtain expression data from sequencing data. Process 2300 may be performed by any suitable computing device or devices, as aspects of the technology described herein are not limited in this respect. For example, process 2300 may be performed by a computing device part of a sequencing platform. In other embodiments, process 2300 may be performed by one or more computing devices external to the sequencing platform.

Process 2300 begins at act 2302, where bulk sequencing data is obtained from a biological sample obtained from a subject. The bulk sequencing data is obtained by any suitable method, for example, using any of the methods described herein including at least with respect to FIG. 1 and in the sections titled “Biological Samples,” “Expression Data,” and “Obtaining Expression Data”.

In some embodiments, the bulk sequencing data obtained at act 2302 comprises RNA-seq data. In some embodiments, the biological sample comprises blood or tissue. In some embodiments, the biological sample comprises one or more tumor cells and one or more TME cells.

Next, process 2300 proceeds to act 2304 where the sequencing data obtained at act 2302 is normalized to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.”, which is incorporated by reference in its entirety herein. In some embodiments, RNA expression level in TPM units for a particular gene may be calculated according to the following formula:

$$A \cdot \frac{1}{\sum ( A )} \cdot {10}^{6} \\ Where A = \frac{total reads mapped to gene \cdot {10}^{3}}{gene legnth in bp} \qquad ( Equation 5 )$$

Next, process 2300 proceeds to act 2306, where the expression levels in TPM units (as determined at act 2304) may be log transformed. Although, in some embodiments, the log transformation is optional and may be omitted.

Process 2300 is illustrative and there are variations. For example, in some embodiments, one or both of acts 2304 and 2306 may be omitted. Thus, in some embodiments, the expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

Expression data obtained by process 2300 can include the sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by process 2300 can include information included in a FASTA file, a description and/or quality scores included in a FASTQ file, an aligned position included in a BAM file, and/or any other suitable information obtained from any suitable file.

Methods of Treatment

In certain methods described herein, an effective amount of anti-cancer therapy described herein may be administered or recommended for administration to a subject (e.g., a human) in need of the treatment via a suitable route (e.g., intravenous administration).

The subject to be treated by the methods described herein may be a human patient having, suspected of having, or at risk for a cancer. Examples of a cancer include, but are not limited to, melanoma, lung cancer, brain cancer, breast cancer, colorectal cancer, pancreatic cancer, liver cancer, prostate cancer, skin cancer, kidney cancer, bladder cancer, or prostate cancer. At the time of diagnosis, the cancer may be cancer of unknown primary. The subject to be treated by the methods described herein may be a mammal (e.g., may be a human). Mammals include but are not limited to: farm animals (e.g., livestock), sport animals, laboratory animals, pets, primates, horses, dogs, cats, mice, and rats.

A subject having a cancer may be identified by routine medical examination, e.g., laboratory tests, biopsy, PET scans, CT scans, or ultrasounds. A subject suspected of having a cancer might show one or more symptoms of the disorder, e.g., unexplained weight loss, fever, fatigue, cough, pain, skin changes, unusual bleeding or discharge, and/or thickening or lumps in parts of the body. A subject at risk for a cancer may be a subject having one or more of the risk factors for that disorder. For example, risk factors associated with cancer include, but are not limited to, (a) viral infection (e.g., herpes virus infection), (b) age, (c) family history, (d) heavy alcohol consumption, (e) obesity, and (f) tobacco use.

An “effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor formation, tumor growth, molecular category identified for the cancer using the techniques described herein) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays. The dosing regimen (including the therapeutic used) may vary over time.

When the anti-cancer therapeutic agent is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward a cancer.

Alleviating a cancer includes delaying the development or progression of the disease or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given period and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

In some embodiments, the anti-cancer therapeutic agent (e.g., an antibody) described herein is administered to a subject in need of the treatment at an amount sufficient to reduce cancer (e.g., tumor) growth by at least 10% (e.g., 20%, 30%, 40%, 50%, 60%, 70%, 80%, 90% or greater). In some embodiments, the anti-cancer therapeutic agent (e.g., an antibody) described herein is administered to a subject in need of the treatment at an amount sufficient to reduce cancer cell number or tumor size by at least 10% (e.g., 20%, 30%, 40%, 50%, 60%, 70%, 80%, 90% or more). In other embodiments, the anti-cancer therapeutic agent is administered in an amount effective in altering cancer type. Alternatively, the anti-cancer therapeutic agent is administered in an amount effective in reducing tumor formation or metastasis.

Conventional methods, known to those of ordinary skill in the art of medicine, may be used to administer the anti-cancer therapeutic agent to the subject, depending upon the type of disease to be treated or the site of the disease. The anti-cancer therapeutic agent can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, an anti-cancer therapeutic agent may be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (e.g., glycerol, propylene glycol, liquid polyethylene glycol, and the like). For intravenous injection, water soluble anti-cancer therapeutic agents can be administered by the drip method, whereby a pharmaceutical formulation containing the antibody and a physiologically acceptable excipients is infused. Physiologically acceptable excipients may include, for example, 5% dextrose, 0.9% saline, Ringer's solution, and/or other suitable excipients. Intramuscular preparations, e.g., a sterile formulation of a suitable soluble salt form of the anti-cancer therapeutic agent, can be dissolved and administered in a pharmaceutical excipient such as Water-for-Injection, 0.9% saline, and/or 5% glucose solution.

In one embodiment, an anti-cancer therapeutic agent is administered via site-specific or targeted local delivery techniques. Examples of site-specific or targeted local delivery techniques include various implantable depot sources of the agent or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568, the contents of each of which are incorporated by reference herein for this purpose.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. The contents of each of the foregoing are incorporated by reference herein for this purpose.

Therapeutic compositions containing a polynucleotide may be administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

Therapeutic polynucleotides and polypeptides can be delivered using gene delivery vehicles. The gene delivery vehicle can be of viral or non-viral origin (e.g., Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). The contents of each of the foregoing are incorporated by reference herein for this purpose. Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed. The contents of each of the foregoing are incorporated by reference herein for this purpose.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581. The contents of each of the foregoing are incorporated by reference herein for this purpose.

It is also apparent that an expression vector can be used to direct expression of any of the protein-based anti-cancer therapeutic agents (e.g., anti-cancer antibody). For example, peptide inhibitors that are capable of blocking (from partial to complete blocking) a cancer-causing biological activity are known in the art.

In some embodiments, more than one anti-cancer therapeutic agent, such as an antibody and a small molecule inhibitory compound, may be administered to a subject in need of the treatment. The agents may be of the same type or different types from each other. At least one, at least two, at least three, at least four, or at least five different agents may be co-administered. Generally anti-cancer agents for administration have complementary activities that do not adversely affect each other. Anti-cancer therapeutic agents may also be used in conjunction with other agents that serve to enhance and/or complement the effectiveness of the agents.

Treatment efficacy can be assessed by methods well-known in the art, e.g., monitoring tumor growth or formation in a patient subjected to the treatment. Alternatively or in addition to, treatment efficacy can be assessed by monitoring tumor type over the course of treatment (e.g., before, during, and after treatment).

A subject having cancer may be treated using any combination of anti-cancer therapeutic agents or one or more anti-cancer therapeutic agents and one or more additional therapies (e.g., surgery and/or radiotherapy). The term combination therapy, as used herein, embraces administration of more than one treatment (e.g., an antibody and a small molecule or an antibody and radiotherapy) in a sequential manner, that is, wherein each therapeutic agent is administered at a different time, as well as administration of these therapeutic agents, or at least two of the agents or therapies, in a substantially simultaneous manner.

Sequential or substantially simultaneous administration of each agent or therapy can be affected by any appropriate route including, but not limited to, oral routes, intravenous routes, intramuscular, subcutaneous routes, and direct absorption through mucous membrane tissues. The agents or therapies can be administered by the same route or by different routes. For example, a first agent (e.g., a small molecule) can be administered orally, and a second agent (e.g., an antibody) can be administered intravenously.

As used herein, the term “sequential” means, unless otherwise specified, characterized by a regular sequence or order, e.g., if a dosage regimen includes the administration of an antibody and a small molecule, a sequential dosage regimen could include administration of the antibody before, simultaneously, substantially simultaneously, or after administration of the small molecule, but both agents will be administered in a regular sequence or order. The term “separate” means, unless otherwise specified, to keep apart one from the other. The term “simultaneously” means, unless otherwise specified, happening or done at the same time, i.e., the agents are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents or therapies described herein.

Combination therapy can also embrace the administration of the anti-cancer therapeutic agent (e.g., an antibody) in further combination with other biologically active ingredients (e.g., a vitamin) and non-drug therapies (e.g., surgery or radiotherapy).

It should be appreciated that any combination of anti-cancer therapeutic agents may be used in any sequence for treating a cancer. The combinations described herein may be selected on the basis of a number of factors, which include but are not limited to reducing tumor formation or tumor growth, and/or alleviating at least one symptom associated with the cancer, or the effectiveness for mitigating the side effects of another agent of the combination. For example, a combined therapy as provided herein may reduce any of the side effects associated with each individual members of the combination, for example, a side effect associated with an administered anti-cancer agent.

In some embodiments, an anti-cancer therapeutic agent is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor, a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

Computer Implementation

An illustrative implementation of a computer system 2400 that may be used in connection with any of the embodiments of the technology described herein (e.g., such as the methods of FIGS. 2A-2C) is shown in FIG. 24. The computer system 2400 includes one or more processors 2410 and one or more articles of manufacture that comprise non-transitory computer-readable storage media (e.g., memory 2420 and one or more non-volatile storage media 2430). The processor 2410 may control writing data to and reading data from the memory 2420 and the non-volatile storage device 2430 in any suitable manner, as the aspects of the technology described herein are not limited to any particular techniques for writing or reading data. To perform any of the functionality described herein, the processor 2410 may execute one or more processor-executable instructions stored in one or more non-transitory computer-readable storage media (e.g., the memory 2420), which may serve as non-transitory computer-readable storage media storing processor-executable instructions for execution by the processor 2410.

Computing device 2400 may also include a network input/output (I/O) interface 2440 via which the computing device may communicate with other computing devices (e.g., over a network), and may also include one or more user I/O interfaces 2450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for using machine learning to estimate tumor expression levels of genes in tumor cells in a biological sample of a subject having cancer, the biological sample comprising the tumor cells and tumor microenvironment (TME) cells, the method comprising:
obtaining expression data for a set of genes, the set of genes comprising a first plurality of genes associated with the tumor cells and a second plurality of genes associated with the tumor microenvironment cells, the expression data comprising first total expression levels for genes in the first plurality of genes and second total expression levels for genes in the second plurality of genes;
determining the tumor expression levels of the first plurality of genes in the tumor cells using a plurality of machine learning models, the plurality of machine learning models comprising a respective machine learning model for each gene in the first plurality of genes including a first machine learning model for a first gene in the first plurality of genes, the tumor expression levels including a first tumor expression level for the first gene in the tumor cells, the determining comprising:
generating a first set of features for the first gene, the generating including:
obtaining, using the expression data, an initial expression level estimate of the first gene in the tumor cells of the biological sample and including the initial expression level estimate of the first gene in the first set of features;
including at least some of the first total expression levels in the first set of features; and
including at least some of the second total expression levels in the first set of features;
providing the first set of features as input to the first machine learning model to obtain an output indicative of a TME expression level estimate of the first gene in the TME cells; and
determining the first tumor expression level for the first gene in the tumor cells using the output of the first machine learning model and a total expression level, in the first total expression levels, for the first gene; and
outputting the tumor expression levels of the first plurality of genes in the tumor cells.

2. The method of claim 1,
wherein the plurality of machine learning models includes a second machine learning model for a second gene in the first plurality of genes and the tumor expression levels include a second tumor expression level for the second gene in the tumor cells, wherein the second machine learning model is different from the first machine learning model and wherein the second gene is different from the first gene, and
wherein determining the tumor expression levels of the first plurality of genes in the tumor cells further comprises:
generating a second set of features for the second gene;
providing the second set of features as input to the second machine learning model to obtain an output indicative of a TME expression level estimate of the second gene in the TME cells; and
determining the second tumor expression level for the second gene in the tumor cells using the output of the second machine learning model and a total expression level, in the first total expression levels, for the second gene.

3. The method of claim 2, wherein generating the second set of features for the second gene comprises:
obtaining, using the expression data, an initial expression level estimate of the second gene in the tumor cells of the biological sample and including the initial expression level estimate of the second gene in the second set of features;
including at least some of the first total expression levels in the second set of features; and
including at least some of the second total expression levels in the second set of features.

4. The method of claim 2,
wherein the plurality of machine learning models includes a third machine learning model for a third gene in the first plurality of genes and the tumor expression levels include a third tumor expression level for the third gene in the tumor cells, wherein the third machine learning model is different from the first machine learning model and from the second machine learning model, wherein the third gene is different from the second gene and from the first gene, and
wherein determining the tumor expression levels of the first plurality of genes in the tumor cells further comprises:
generating a third set of features for the third gene;
providing the third set of features as input to the third machine learning model to obtain an output comprising a TME expression level estimate of the third gene in the TME cells; and
determining the third tumor expression level for the third gene in the tumor cells using the output of the third machine learning model and a total expression level, in the first total expression levels, for the third gene.

5. The method of claim 1, wherein generating the first set of features for the first gene further comprises:
obtaining, using the expression data, a first plurality of RNA percentages for a respective plurality of types of cells that occur in the TME, wherein each of the first plurality of RNA percentages indicates a percent of RNA associated with the first gene and originating from cells of a respective type in the TME in the biological sample.

6. The method of claim 5, wherein generating the first set of features for the first gene further comprises including at least some of the first plurality of RNA percentages in the first set of features.

7. The method of claim 5, wherein obtaining the first plurality of RNA percentages comprises processing at least some of the expression data using at least one non-linear regression model.

8. The method of claim 7,
wherein the TME cells comprise TME cells of a first type and TME cells of a second type,
wherein the at least some of the expression data includes a first subset of the expression data and a second subset of the expression data,
wherein the at least one non-linear regression model includes a first non-linear regression model and a second non-linear regression model different from the first non-linear regression model, and
wherein obtaining the first plurality of RNA percentages comprises:
processing the first subset of the expression data using the first non-linear regression model to obtain a first RNA percentage for the TME cells of the first type; and
processing the second subset of the expression data using the second non-linear regression model to obtain a second RNA percentage for the TME cells of the second type.

9. The method of claim 8,
wherein the first type and the second type are each selected from the group consisting of B cells, CD4+ T cells, CD8+ T cells, endothelial cells, fibroblasts, lymphocytes, macrophages, monocytes, NK cells, and neutrophils, wherein the first type is different from the second type.

10. The method of claim 5, wherein obtaining the initial expression level estimate of the first gene in the tumor cells of the biological sample comprises:
obtaining an average TME expression level of the first gene for each of the plurality of types of cells that occur in the TME;
determining a weighted sum of the obtained expression levels based on the first plurality of RNA percentages; and
subtracting the weighted sum from the total expression level for the first gene to obtain the initial expression level estimate.

11. The method of claim 1, further comprising:
obtaining, using the expression data, a first RNA percentage for the tumor cells, wherein the first RNA percentage indicates a percent of RNA associated with the first gene and originating from the tumor cell of the biological sample.

12. The method of claim 11, wherein determining the first tumor expression level for the first gene in the tumor cells further comprises:
subtracting the TME expression level estimate from the total expression level for the first gene; and
dividing a result of the subtracting by the first RNA percentage.

13. The method of claim 1, wherein the expression data has been previously obtained at least in part by sequencing the biological sample of the subject having cancer.

14. The method of claim 1,
wherein the at least some of the first total expression levels included in the first set of features include total expression levels for at least 25 genes in the first plurality of genes associated with the tumor cells, and
wherein the plurality of machine learning models comprises at least 25 machine learning models corresponding to the at least 25 genes.

15. The method of claim 14, wherein each machine learning model of the at least 25 machine learning models comprises a different gradient boost model.

16. The method of claim 1,
wherein the at least some of the first total expression levels included in the first set of features include total expression levels for at least 10 genes selected from genes listed in Table 1, wherein Table 1 comprises:
TABLE 1
Genes Associated with Tumor Cells

| | |
|---|---|
| NF1 | NM_001042492; NM_000267; NM_001128147 |
| CCNE1 | XM_011527440; NM_001238; NM_001322259; |
| | NM_001322261; XM_047439606; NM_001322262; |
| | NM_057182 |
| PLK1 | NM_005030 |
| ERBB4 | XM_005246376; XM_017003577; XM_017003578; |
| | XM_005246377; NM_001042599; XM_017003581; |
| | XM_006712364; XM_017003582; XM_017003579; |
| | XM_017003580; NM_005235 |
| NF2 | XM_047441386; NM_181828; NM_181830; NM_181826; |
| | NM_000268; NR_156186; NM_181827; NM_181834; |
| | NM_016418; NM_181829; NM_181825; NM_181831; |
| | NM_181835; XM_017028809; NM_181832; NM_181833 |
| XRCC1 | NM_006297 |
| MAGEA1 | NM_004988 |
| PDGFA | XM_011515415; XM_011515419; XM_011515418; |
| | NM_001395365; NR_172526; XM_011515416; |
| | XM_047420455; XM_047420458; NM_001395363; |
| | NM_001395364; NM_033023; XM_017012289; |
| | NM_001395366; XM_047420457; NR_172527; |
| | XM_047420456; NM_002607 |
| HDAC2 | NR_033441; XM_047418692; NR_073443; NM_001527 |
| BCL2L2 | NM_004050; NM_001199839 |
| NOTCH3 | XM_005259924; NM_000435 |
| TUBB3 | NM_006086; NM_001197181 |
| AURKB | NM_001313950; NM_001313953; XM_017025311; |
| | XM_047437050; NM_001313952; NM_004217; |
| | NM_001313954; NR_132730; NR_132731; |
| | NM_001284526; XM_047437051; XM_011524072; |
| | NM_001256834; NM_001313951; NM_001313955 |
| CCND2 | NM_001759 |
| CDKN2A | XM_011517676; XM_011517675; NM_001363763; |
| | NM_001195132; XM_047422597; NM_058195; |
| | XM_047422596; XM_047422598; NM_000077; |
| | NM_058196; NM_058197 |
| CCNE2 | XM_047422411; XM_017013958; NM_057749; |
| | XM_011517366; XM_017013959; NM_004702; |
| | NM_057735 |
| ROR2 | XM_005252008; XM_017014762; XM_047423434; |
| | XM_047423436; XM_006717121; XM_047423435; |
| | NM_004560; XM_005252009; XM_047423437; |
| | NM_001318204 |
| RRM2 | NM_001034; NR_164157; NR_161344; NM_001165931 |
| UMPS | NR_033437; XR_001740253; NR_033434; NM_000373 |
| CIITA | XM_047434115; NM_001379332; XR_007064880; |
| | XM_006720880; XM_011522491; XM_047434119; |
| | NM_001379334; XM_047434118; XM_047434120; |
| | XM_047434123; NM_001379333; XM_011522486; |
| | NM_000246; NM_001286402; XM_047434122; |
| | XM_047434126; XR_001751904; XR_007064879; |
| | XM_047434114; XM_047434117; XM_047434125; |
| | NM_001286403; NM_001379331; XM_011522485; |
| | XM_047434127; XM_047434128; NR_104444; |
| | XM_011522484; XM_011522490; XM_047434116; |
| | XM_047434124; NM_001379330 |
| HDAC4 | XM_011512219; XM_011512225; XM_047446479; |
| | XM_047446483; XM_047446487; NM_001378415; |
| | XM_011512218; XM_017005394; XM_047446484; |
| | XM_047446490; XM_047446492; XM_047446494; |
| | XM_011512224; XM_047446477; XM_047446478; |
| | XM_047446480; XM_047446493; XM_047446496; |
| | NM_001378416; NM_006037; XM_011512223; |
| | XM_011512227; XM_047446482; NM_001378414; |
| | XM_011512220; XM_011512222; XM_024453257; |
| | XM_047446485; XM_047446486; XM_047446489; |
| | XM_047446495; XM_011512217; XM_011512226; |
| | XM_047446476; XM_047446491; XM_047446497; |
| | XM_047446498; NM_001378417; XM_006712877; |
| | XM_006712880; XM_047446481; XM_047446488 |
| DPYD | XM_006710397; XM_017000507; XM_047448077; |
| | NM_000110; NM_001160301; XM_047448076; |
| | XR_001737014; XM_005270562 |
| AKT2 | XM_011526616; XM_047438397; NM_001626; |
| | XM_047438398; XM_047438403; XM_011526619; |
| | XM_047438399; XM_047438401; NM_001243027; |
| | XM_011526618; NM_001243028; NM_001330511; |
| | XM_011526614; XM_047438400; XM_047438402; |
| | XM_011526615 |
| PIK3CD | XM_024447663; XM_047422552; XM_047422561; |
| | XM_047422568; XM_047422573; XM_047422574; |
| | XM_047422575; XM_047422577; XM_024447664; |
| | XM_047422553; XM_047422564; XM_047422566; |
| | NM_005026; XM_047422567; XM_047422569; |
| | NM_001350234; XM_047422554; XM_047422555; |
| | XM_047422589; XM_006710689; XM_047422550; |
| | XM_047422557; XM_006710687; XM_047422558; |
| | XM_047422559; XM_047422563; XM_047422565; |
| | XM_047422580; XM_047422551; XM_047422556; |
| | XM_047422562; XM_047422570; XM_047422571; |
| | NM_001350235; XM_047422560; XM_047422572; |
| | XM_047422576; XM_047422578 |
| AURKA | XM_047440427; XM_047440428; NM_001323304; |
| | NM_001323303; NM_198435; NM_198437; NM_198433; |
| | NM_198434; NM_198436; XM_017028034; |
| | XM_017028035; NM_001323305; NM_003600 |
| ATR | XM_047448362; XM_011512925; NM_001354579; |
| | XM_047448361; XM_011512924; XM_047448363; |
| | NM_001184; XM_047448364; XM_047448360 |
| EREG | NM_001432 |
| FGFR1 | XM_024447097; XM_047421569; XM_047421570; |
| | NM_001174065; NM_001354370; NM_023111; |
| | XM_006716303; XM_006716304; XM_006716310; |
| | XM_011544445; XM_011544449; XM_017013221; |
| | XM_017013225; NM_001354368; NM_001354369; |
| | NM_015850; NM_023106; XM_006716307; |
| | XM_011544444; XM_047421571; XM_047421572; |
| | NM_001354367; NM_023105; XM_00671631 1; |
| | XM_011544446; XM_011544452; XM_017013219; |
| | XM_017013226; XM_047421573; XM_047421574; |
| | NM_023107; NM_023109; XM_011544447; |
| | XM_011544451; NM_023110; XM_006716312; |
| | XM_011544450; XM_017013220; XM_017013227; |
| | XM_017013231; NM_001174067; NM_032191; |
| | XM_006716314; XM_011544448; XM_047421575; |
| | NM_001174063; NM_001174064; NM_001174066; |
| | XM_047421576; NM_023108 |
| HDAC9 | NM_001204147; NM_001321868; NM_001321878; |
| | NM_001321887; NM_001321891; NM_001321897; |
| | NM_058177; NM_001204144; NM_001321873; |
| | NM_001321879; NM_001321884; NR_135835; |
| | NM_001321890; NM_001321894; NM_001321898; |
| | NM_001321900; NM_014707; NM_178425; |
| | NM_001321874; NM_001321877; NM_001321888; |
| | NM_001321895; NM_058176; NM_001321869; |
| | NM_001321885; NM_001321886; NM_001321899; |
| | NM_001321901; NM_001321902; NM_178423; |
| | NM_001204146; NM_001204148; NM_001321870; |
| | NM_001321893; NM_001321871; NM_001321875; |
| | NM_001204145; NM_001321872; NM_001321876; |
| | NM_001321889; NM_001321896 |
| MAGEA2 | NM_001386130.2; NM_005361.3; NM_175742.2; |
| | NM_175743.2; NM_001282501.2; NM_001282502.1; |
| | NM_001282504.1; NM_001282505.1 |
| FLNA | NM_001110556.2; NM_001456.4 |
| SLC39A6 | NM_001099406; NM_012319 |
| FLT1 | NM_001160030; NM_001159920; XM_011535014; |
| | XM_017020485; NM_001160031; NM_002019 |
| CD22 | NM_001185100; NM_001185099; NM_024916; |
| | NM_001185101; NM_001771; NM_001278417 |
| ALK | NM_004304; NM_001353765; XR_001738688 |
| PGR | XM_011542869; NM_001271161; NR_073142; |
| | XM_006718858; NM_000926; NM_001202474; |
| | NM_001271162; NR_073141; NR_073143 |
| TP53 | NM_000546; NM_001126112; NM_001276695; |
| | NM_001126115; NM_001126116; NM_001126118; |
| | NM_001276697; NM_001276698; NM_001276760; |
| | NM_001276761; NM_001126114; NM_001276696; |
| | NM_001126113; NM_001126117; NM_001276699 |
| FGFR2 | XM_017015924; NM_001144919; XM_006717708; |
| | XM_017015925; NM_001144915; NM_001144917; |
| | NM_022975; NM_023028; XM_024447890; NM_000141; |
| | NM_001144913; NM_001320654; NM_022970; |
| | NR_073009; NM_022971; NM_022973; NM_023030; |
| | XM_006717710; XM_024447887; XM_024447888; |
| | NM_001320658; NM_022976; XM_017015920; |
| | NM_001144918; NM_022974; NM_023031; |
| | XM_024447889; XM_024447891; NM_023029; |
| | XM_017015921; NM_001144914; NM_001144916; |
| | NM_022972 |
| TXNRD1 | NM_001261446; NM_182742; NM_182743; NM_003330; |
| | NM_182729; NM_001093771; NM_001261445 |
| STK11 | NM_000455 |
| MAGEA3 | XM_011531161; XM_005274676; XM_006724818; |
| | XM_011531160; NM_005362 |
| CDKN1A | NM_001220778; NM_001374510; NM_078467; |
| | NR_164655; NM_001291549; NM_001374511; |
| | NM_001374509; NR_164656; NM_000389; |
| | NM_001220777; NM_001374512; NM_001374513 |
| MAGEA4 | NM_001386196; NM_001386197; NM_001386200; |
| | NM_002362; NM_001011550; NM_001386202; |
| | NM_001011548; NM_001011549; NM_001386198; |
| | NM_001386203; NM_001386199 |
| NTRK3 | XM_006720550; XR_001751292; XM_024449935; |
| | XM_047432602; NM_001375813; XR_002957645; |
| | XM_017022245; XM_017022252; XM_024449934; |
| | NM_001375812; XM_006720549; XM_017022241; |
| | XM_017022250; NM_001320135; XM_017022240; |
| | XM_047432603; NM_001012338; XM_006720545; |
| | XM_011521638; XM_017022244; XM_017022251; |
| | XM_047432604; NM_001007156; NM_001243101; |
| | XM_017022242; NM_001320134; NM_001375810; |
| | NM_001375814; NM_002530; XM_006720548; |
| | XM_017022243; XM_017022254; NM_001375811; |
| | XR_001751293 |
| TERT | NR_149162; NM_198255; NM_198253; NR_149163; |
| | NM_001193376; NM_198254 |
| CDK4 | NM_000075; NM_052984 |
| XRCC5 | NM_021141 |
| B2M | XM_005254549; NM_004048 |
| CHEK2 | XM_006724114; XM_011529845; XM_024452148; |
| | XM_047441105; XM_047441106; NM_001349956; |
| | XM_006724116; XR_007067954; XM_017028560; |
| | XM_047441104; NM_001257387; NM_007194; |
| | XM_011529842; XM_047441108; NM_145862; |
| | XM_011529839; XM_011529844; XM_024452149; |
| | XM_047441107; XR_937806; XR_937807; |
| | XM_011529840; NM_001005735; XR_007067955 |
| TSC2 | XM_047434556; NM_021056; NM_001318831; |
| | XM_047434555; XM_011522637; NM_001077183; |
| | NM_001318832; NM_001363528; XM_011522639; |
| | XM_017023615; XM_047434557; NM_001318827; |
| | NM_001370405; XM_011522636; XM_011522640; |
| | NM_000548; NM_001370404; NM_021055; |
| | XM_011522638; NM_001114382; NM_001318829 |
| EGF | XM_017007848; XM_005262796; XM_011531707; |
| | XM_017007850; XM_047449723; NM_001178131; |
| | XM_047449725; XM_017007847; XM_017007855; |
| | XM_047449726; XM_047449727; XM_047449729; |
| | XM_017007854; NM_001963; XR_001741156; |
| | XM_017007845; XM_017007849; XM_047449728; |
| | NM_001178130; XM_017007846; XM_017007853; |
| | NM_001357021; XM_017007851; XM_047449724; |
| | XM_047449730 |
| ABCC3 | NM_001144070; NM_003786; NM_020037; NM_020038 |
| IDO1 | NM_002164 |
| ERBB2 | NM_001005862; NM_001382784; NM_001382785; |
| | NM_001382788; NM_001382792; NM_001382793; |
| | NM_001382803; XM_047435590; NM_001289937; |
| | NM_001382786; NM_001382800; NM_001382802; |
| | NM_001382806; NM_001382782; NM_001382789; |
| | NM_001382795; NM_001289936; NM_001382797; |
| | NM_001382805; NM_004448; NR_110535; |
| | NM_001289938; NM_001382791; NM_001382801; |
| | NM_001382783; NM_001382790; NM_001382794; |
| | NM_001382798; NM_001382799; NM_001382787; |
| | NM_001382796; NM_001382804 |
| HDAC1 | XM_011541309; NM_004964 |
| RAD50 | NM_005732; NM_133482 |
| SMO | NM_005631; XM_047420759 |
| STAT6 | NM_001178078; NM_001178080; NM_001178081; |
| | XM_047429475; NM_001178079; XM_047429476; |
| | XM_047429473; XM_047429477; NM_003153; |
| | XM_047429474; NR_033659 |
| PIK3CA | NM_006218; XM_006713658 |
| HDAC7 | NR_160436; NM_015401; XM_011538481; |
| | XM_024449018; XM_047428978; NM_001308090; |
| | NM_016596; XM_011538483; XM_047428981; |
| | NR_160435; XM_047428979; XM_047428984; |
| | XM_011538480; XM_047428980; XM_047428982; |
| | XM_047428983; NM_001098416; NM_001368046 |
| IGF1R | XM_047432444; XM_011521517; NM_000875; |
| | XM_011521516; XM_017022137; XM_047432442; |
| | NM_152452; XM_047432443; XM_047432445; |
| | NM_001291858 |
| IGF1 | XM_017019263; XM_017019261; XM_017019262; |
| | XM_017019259; NM_001111284; NM_001111285; |
| | NM_001111283; NM_000618 |
| ICAM1 | NM_000201 |
| ROS1 | XM_011536053; XM_011536055; XM_011536054; |
| | XM_011536057; XM_011536049; XM_011536058; |
| | NM_001378891; XM_047419232; XM_006715548; |
| | NM_002944; XM_011536050; XM_017011173; |
| | XM_047419231; XM_011536051; XM_011536056; |
| | XM_017011172; NM_001378902 |
| MCL1 | NM_001197320; NM_182763; NM_021960 |
| TACSTD2 | NM_002353 |
| NRAS | NM_002524 |
| CCND1 | NM_053056 |
| XRCC3 | XM_005268046; NM_001371231; XM_047431767; |
| | XM_047431768; NM_001100119; NM_001371229; |
| | XM_047431766; NM_001371232; NM_001100118; |
| | NM_005432 |
| MKI67 | NM_002417; NM_001145966; XM_006717864; |
| | XM_011539818 |
| EPHA2 | XM_017000537; XM_047448267; XM_047448259; |
| | NM_001329090; XM_047448272; NM_004431 |
| BCL6 | NM_001130845; XM_011513062; NM_001706; |
| | XM_047448655; NM_001134738; NM_138931; |
| | XM_005247694 |
| BCL2L1 | XM_047440353; NM_001317919; NM_001322240; |
| | NM_001322242; XM_011528964; XM_047440351; |
| | NM_001191; NM_001317920; NR_134257; |
| | XM_017027993; NM_001317921; NM_138578; |
| | XM_047440352; NM_001322239 |
| ATF3 | XM_047421211; NM_001206488; NM_001674; |
| | NM_001206484; NM_004024; XM_005273146; |
| | NM_001040619; NM_001206486; NM_001030287; |
| | XM_011509579; NM_001206485 |
| MAGEA12 | NM_001166386; NM_001166387; NM_005367 |
| FGFR3 | XM_047449823; XM_047449824; XM_006713869; |
| | XM_006713873; NM_022965; XM_006713868; |
| | NM_001354810; XM_011513422; XM_047449821; |
| | XM_047449822; NM_000142; XM_011513420; |
| | XM_047449820; XM_006713870; XM_006713871; |
| | NM_001163213; NM_001354809; NR_148971 |
| DLL3 | NM_016941; NM_203486 |
| AREG | NM_001657 |
| PMEL | NM_001200054; NM_001200053; NM_001320121; |
| | NM_001384361; NM_001320122; NM_006928 |
| PDCD1LG2 | XM_005251600; NM_025239 |
| TPBG | NM_001166392; NM_001376922; NM_006670 |
| ATM | XM_011542844; XM_047426976; XM_047426978; |
| | NM_001351834; XM_011542840; XM_011542842; |
| | XM_047426975; NM_138293; XM_005271562; |
| | XM_006718843; XM_047426979; NM_000051; |
| | NM_001351835; XM_006718845; XM_047426981; |
| | NM_001351836; XM_011542843; XM_017017790; |
| | XM_047426977; NM_138292 |
| PIK3CG | XM_017012328; XM_005250443; XM_047420479; |
| | NM_001282426; XM_011516317; XM_047420481; |
| | XM_047420480; NM_001282427; XM_011516316; |
| | NM_002649 |
| RRM1 | NM_001033; NM_001330193; NM_001318065; |
| | NM_001318064 |
| INSR | NM_001079817; NM_000208; XM_011527989; |
| | XM_011527988 |
| CDH1 | NM_001317186; NM_004360; NM_001317185; |
| | NM_001317184 |
| KMT2C | NM_170606; NM_021230 |
| CA9 | XM_047423849; NM_001216; XM_047423850 |
| IGF2R | NM_000876 |
| CD274 | XM_047423262; NM_001314029; NM_001267706; |
| | NR_052005; NM_014143 |
| ADORA2B | XM_017024197; XM_011523661; XM_047435375; |
| | NM_000676; XM_047435374; XM_011523659; |
| | XM_047435373 |
| BIRC5 | NM_001168; NM_001012270; NM_001012271 |
| TYMS | NM_001354867; NM_001354868; XM_024451242; |
| | NM_001071 |
| MUC1 | NM_001018017; NM_001044391; NM_001044393; |
| | NM_001204291; NM_001044390; NM_001204285; |
| | NM_182741; NM_001371720; NM_001204289; |
| | NM_001204290; NM_001204293; NM_001018016; |
| | NM_001044392; NM_001204286; NM_001204287; |
| | NM_001204288; NM_001204295; NM_001018021; |
| | NM_001204292; NM_001204294; NM_001204297; |
| | NM_001204296; NM_002456 |
| MYB | NM_001161660; NR_134958; NM_001130172; |
| | NM_001130173; NM_001161656; NR_134959; |
| | NM_001161657; XM_047418834; NR_134963; |
| | NR_134965; NR_134962; XR_942444; NM_001161659; |
| | NR_134961; NM_001161658; NM_005375; NR_134960; |
| | NR_134964 |
| CCND3 | XM_047419491; NM_001287434; NM_001136017; |
| | NM_001760; NM_001136125; NM_001136126; |
| | XM_011514971; NM_001287427 |
| RB1 | NM_000321 |
| TOP1 | NM_003286 |
| MMP2 | NM_001302509; NM_001127891; NM_001302508; |
| | NM_001302510; NM_004530 |
| PTEN | NM_000314; NM_001304718; NM_001304717 |
| FN1 | NM_001306129; NM_001365519; NM_212474; |
| | NM_001306132; NM_001365517; NM_001365522; |
| | NM_001306131; NM_001365521; NM_212476; |
| | NM_212478; NM_212475; NM_001365523; |
| | NM_001365524; NM_002026; NM_001365520; |
| | NM_212482; NM_001365518; NM_054034; |
| | NM_001306130 |
| BRAF | XM_047420766; XM_047420768; NM_001374244; |
| | NM_001374258; NM_001378471; NM_001378473; |
| | NR_148928; XM_047420767; XM_047420769; |
| | XM_047420770; NM_001378467; NM_001378468; |
| | XM_017012559; NM_001378470; NM_001378472; |
| | NM_001378475; NM_001354609; NM_001378469; |
| | NM_001378474; NM_004333 |
| KMT2E | XM_047420611; NM_018682; XM_005250493; |
| | NM_032187; XM_047420613; XM_011516400; |
| | XM_047420612; NM_182931 |
| FGFR4 | NM_213647; NM_022963; NM_002011; NM_001291980; |
| | NM_001354984 |
| BRCA1 | NM_007299; NM_007303; NM_007294; NM_007306; |
| | NM_007298; NM_007295; NM_007301; NM_007300; |
| | NR_027676; NM_007305; NM_007296; NM_007297; |
| | NM_007302 |
| ERBB3 | XM_047428500; NM_001005915; XM_047428501; |
| | NM_001982 |
| CEACAM6 | NM_002483; XM_011526990 |
| EPCAM | NM_002354 |
| SMARCA4 | XM_024451667; NM_001128845; NM_001387283; |
| | NR_164683; XM_047439249; NM_001128848; |
| | XM_047439243; XM_047439246; XM_047439247; |
| | XM_047439251; XM_006722846; XM_024451661; |
| | XM_047439245; NM_001374457; XM_047439250; |
| | NM_001128846; XM_011528198; XM_024451663; |
| | NM_001128847; XM_047439244; NM_001128844; |
| | NM_001128849; NM_003072; XM_024451658; |
| | XM_047439248 |
| BRCA2 | NM_000059 |
| MTOR | NM_001386501; XM_017000900; XM_011541166; |
| | NM_001386500; XR_007058581; XM_047416721; |
| | XM_047416724; NM_004958 |
| CDK2 | NM_001290230; XM_011537732; NM_052827; |
| | NM_001798 |
| PTK7 | NM_152880; NM_152882; NM_152881; XM_047419157; |
| | NM_002821; NR_072997; NR_072998; NM_152883; |
| | NM_001270398; XM_011514766; XM_011514765 |
| EGFR | XM_047419953; NM_001346899; NM_201282; |
| | XM_047419952; NM_201284; NM_001346898; |
| | NM_001346900; NM_001346897; NM_201283; |
| | NM_001346941; NM_005228 |
| STMN1 | NM_203399; NM_203401; NM_152497; NM_005563; |
| | NM_001145454 |
| ADORA1 | NM_001048230; XM_047446499; NM_000674; |
| | NM_001365065; NM_001365066 |
| NAE1 | XM_047434835; NM_001018160; NM_003905; |
| | NM_001286500; NM_001018159 |
| IGF2 | NM_001291862; NM_001291861; NM_000612; |
| | NM_001007139; NM_001127598 |
| IRF2 | NM_002199 |
| ABCB1 | NM_001348946; NM_001348944; NM_000927; |
| | NM_001348945 |
| WT1 | NM_000378; NR_160306; NM_001367854; |
| | NM_001198551; NM_001198552; NM_024424; |
| | NM_024426; NM_024425 |
| MDM2 | NM_006880; NM_006882; XM_047428853; NM_006878; |
| | NM_001145340; NM_001278462; NM_001367990; |
| | NM_006879; NM_001145337; NM_002392; |
| | NM_006881; NM_032739; NM_001145339; |
| | NM_001145336 |
| MAGEA10 | NM_001251828; NM_021048; NM_001011543 |
| ERCC1 | NM_001369419; NM_001369409; NM_001166049; |
| | NM_001369412; NM_001369417; NM_202001; |
| | NM_001369415; NM_001369418; NM_001369408; |
| | NM_001369410; NM_001369411; NM_001369413; |
| | NM_001369414; NM_001369416; NM_001983 |
| ADORA2A | NM_000675; NR_103544; NM_001278498; |
| | NM_001278499; NM_001278500; NR_103543; |
| | NM_001278497 |
| KRAS | XM_047428826; NM_001369786; NM_033360; |
| | NM_004985; NM_001369787 |
| ITGB4 | XM_047435927; XM_005257311; XM_006721866; |
| | XM_006721870; NM_000213; NM_001005619; |
| | NM_001005731; XM_005257309; XM_011524752; |
| | XM_006721867; XM_011524751; XM_047435929; |
| | NM_001321123; XM_047435926; XM_047435928; |
| | XM_006721868 |

17. The method of claim 1,
wherein the at least some of the first total expression levels included in the first set of features include total expression levels for at least 25 genes selected from genes listed in Table 1.

18. The method of claim 1,
wherein the at least some of the first total expression levels included in the first set of features include total expression levels for at least 50 genes selected from genes listed in Table 1.

19. The method of claim 1,
wherein the at least some of the first total expression levels included in the first set of features include total expression levels for at least 75 genes selected from genes listed in Table 1.

20. The method of claim 1, wherein the first machine learning model of the plurality of machine learning models is a gradient boosted model.

21. The method of claim 1, further comprising training the first machine learning by:
obtaining training data comprising simulated expression data for genes in the set of genes, wherein the training data is associated with one or more biological samples; generating, using the training data, a training set of features for the first gene;
training the first machine learning model to estimate a TME expression level of the first gene, the training comprising:
providing the training set of features as input to the first machine learning model to obtain an output comprising an estimate of the TME expression level of the first gene in the TME cells of the one or more biological samples; and
updating parameters of the first machine learning model using the estimate of the TME expression level.

22. The method of claim 21, wherein generating the training set of features for the first gene comprises:
obtaining, using the simulated expression data, an initial expression level estimate of the first gene in tumor cells of the one or more biological samples and including the initial expression level estimate in the training set of features; and
including at least some of the simulated expression levels in the training set of features.

23. The method of claim 1, wherein the first machine learning model was trained at least in part by generating training data comprising simulated expression data, wherein generating the training data comprises:
obtaining training expression data for each of one or more biological samples, the training expression data comprising first training expression levels for the first plurality of genes and second training expression levels for the second plurality of genes;
generating first simulated expression data using the first training expression levels;
generating second simulated expression data using the second training expression levels; and
combining the first simulated expression data and the second simulated expression data to produce at least part of the simulated expression data.

24. The method of claim 1, further comprising:
identifying at least one anti-cancer therapy for the subject based on the first tumor expression level for the first gene in the tumor cells.

25. The method of claim 24, further comprising:
administering the at least one anti-cancer therapy.

26. The method of claim 24, wherein the at least one anti-cancer therapy is selected from the group of therapies for the first gene listed in Table 3, wherein Table 3 comprises:

| Gene | Cancer Types | Therapy |
|---|---|---|
| ALK | anaplastic large-cell lymphoma, | Crizotinib |
| | inflammatory myofibroblastic tumors, | |
| | diffuse large B-cell lymphoma, | |
| | non-small-cell lung cancer (NSCLC), | |
| | colorectal, breast carcinomas | |
| PTK7 | atypical teratoid rhabdoid tumors, | PTK7 Antibody-drug |
| | breast cancer, cholangiocarcinoma, | conjugate, PF-06647020 |
| | colorectal cancer, esophageal | |
| | squamous cell carcinoma and gastric | |
| | cancer, cholangiocarcinoma | |
| PIK3CG | colorectal cancers, | Combination of |
| | colon cancers, | paclitaxel (PTX) and |
| | claudin-low breast cancer | AS-605240 |
| CDH1 | hereditary diffuse gastric cancer, | Suppressor-tRNA |
| | lobular breast cancer | |
| MKI67 | bladder cancer, CNS and brain, breast | Ki-67 labeling index for |
| | cancer (BC), colorectal cancer (CRC), | diagnosis and prognosis |
| | cervical cancer, esophageal cancer | assessment of cancer |
| | (EC), head and neck cancer (HNC), | patients |
| | gastric cancer (GC), liver cancer, | |
| | ovarian cancer, lung cancer (LC), | |
| | lymphoma, sarcoma, and pancreatic | |
| | cancer compared with noncarcinoma | |
| | tissues. | |
| CCND2 | triple-negative breast cancer and lung | Antroquinonol D |
| | adenocarcinoma, non-small-cell lung | |
| | carcinoma and breast cancer patients | |
| BCL2L2 | Neoplasm | Inferior response to |
| | | navitoclax in cancer. |
| CDK2 | glioblastoma, prostate cancer, B cell | CDK2 inhibition (using |
| | lymphoma, triple-negative breast | CYC065) combined with |
| | cancer | eribulin. |
| PDGFA | liver cancer, breast cancer, and oral | PDGF receptor kinase |
| | squamous cell carcinoma, | inhibitors imatinib or |
| | neuroblastomas, osteosarcoma, and | sunitinib |
| | gastric carcinoma, papillary thyroid | |
| | cancer, cholangiocarcinoma | |
| IGF2 | colorectal, breast, prostate and lung | MABs that bind IGF2 |
| | cancers, hepatoblastoma | |
| FGFR | squamous cell carcinomas of the lung | Prognostic biomarker, |
| | and the head and neck, glioblastoma, | that correlates with |
| | melanoma, breast, prostate, bladder, | parameters of worse |
| | and ovarian cancer | outcome |
| FLNA | malignant mesothelioma, breast | Therapy or others to |
| | cancer | induce cleavage of |
| | | FLNA |
| TOP1 | colon cancer, breast cancer, ovarian | Top1 targeting drugs, |
| | cancer, and recurrent small-cell lung | Enhancement of |
| | cancer | radiotherapy with TOP1 |
| | | drugs (Camptothecin). |
| KMT2E | large intestine, ovary, central nervous | Prognostic marker for |
| | system, and stomach, but | patients with AML |
| | downregulation in others, e.g., the | treated in the AMLSHG |
| | pancreas, thyroid, and breast cancer | 0199 and AMLSHG |
| | | 0295 trials |
| B2M | breast cancer, prostate cancer, lung | Inhibitors targeting the |
| | cancer, renal cancer, multiple | B2M in combination |
| | myeloma, and especially non- | with other immune |
| | Hodgkin’s lymphoma, colorectal | checkpoint molecules. |
| | cancer | |
| ERBB3 | ovarian, breast, prostate, gastric, | Activation of HER3 |
| | bladder, lung, melanoma, colorectal | signaling is one major |
| | and squamous cell carcinoma, | cause of treatment failure |
| | pancreatic carcinoma | to EGFR or anti- |
| | | estrogenbased therapies. |
| MDM2 | bladder carcinoma, non-Hodgkin's | Diagnostic tool or as a |
| | lymphoma, prostate carcinoma, | marker, particularly for |
| | testicular germ cell tumors, soft tissue | tumor stage or grade. |
| | sarcomas | |
| MCL1 | multiple myeloma, leukemia, non- | Gapil et al. extracted 26 |
| | Hodgkin lymphoma, lung cancer | carboxamides from |
| | | natural fislatifolic acid, |
| | | one of which exhibited |
| | | submicromolar affinity |
| | | for MCL-1 and BCL-2, |
| | | and showed moderate |
| | | cytotoxicity in lung |
| | | and breast cancer cell |
| | | lines |
| MYB | myeloid leukemia (AML), non- | Block gene function |
| | Hodgkin lymphoma, colorectal | with antisense oligo- |
| | cancer, and breast cancer, colon | nucleotides |
| | cancer | |
| AURKA | adrenocortical carcinoma (ACC), | Aurora kinase inhibitors |
| | LGG, KICH, kidney renal clear cell | (e.g., AKI-001, |
| | carcinoma (KIRC), kidney renal | BPR1K871, MLN8054). |
| | papillary cell carcinoma (KIRP), liver | Use in clinical drugs and |
| | hepatocellular carcinoma (LIHC), | in combination with |
| | lung adenocarcinoma (LUAD), | radiotherapy. |
| | mesothelioma (MESO), PAAD, | PHA680632 treatment |
| | SARC and uveal melanoma (UVM). | prior to radiation |
| | | treatment leads to an |
| | | additive effect in cancer |
| | | cells, especially in p53- |
| | | deficient cells in vitro or |
| | | in vivo. |
| PTEN | prostate cancer, breast cancer, | PTEN loss has |
| | glioblastoma, malignant melanoma, | previously been reported |
| | endometrial, prostate, breast, | to be prognostic for |
| | colorectal and pancreatic cancer | outcome following |
| | | radiotherapy in prostate |
| | | cancer. PTEN expression |
| | | also a predictive marker |
| | | for targeted therapeutic |
| | | agents including anti- |
| | | EGFR mAbs, |
| | | trastuzumab-based |
| | | chemotherapy in breast |
| | | cancer. |
| STMN1 | breast cancer, lung cancer, ovarian | A variety of target- |
| | cancer, prostate cancer, sarcoma, and | specific anti-stathmin |
| | gastric cancer | effectors, including |
| | | ribozymes and si-RNA |
| | | have been used to silence |
| | | stathmin in vitro as |
| | | singlets and in |
| | | combination with |
| | | chemotherapeutic agents |
| | | where additive |
| | | synergistic interactions |
| | | have been demonstrated |
| | | (e.g., taxanes) |

27. The method of claim 24, wherein identifying the at least one anti-cancer therapy for the subject comprises:
determining whether the first tumor expression level satisfies at least one criterion associated with the first gene; and
after determining that the first tumor expression level satisfies the at least one criterion, selecting the at least one anti-cancer therapy from the group of therapies listed for the first gene in Table 3.

28. A system, comprising:
at least one processor;
at least one non-transitory computer-readable storage medium storing processor-executable instructions that, when executed by the at least one processor, cause the at least one processor to perform a method for using machine learning to estimate tumor expression levels of genes in tumor cells in a biological sample of a subject having cancer, the biological sample comprising the tumor cells and tumor microenvironment (TME) cells, the method comprising:
obtaining expression data for a set of genes, the set of genes comprising a first plurality of genes associated with the tumor cells and a second plurality of genes associated with the TME cells, the expression data comprising first total expression levels for genes in the first plurality of genes and second total expression levels for genes in the second plurality of genes;
determining the tumor expression levels of the first plurality of genes in the tumor cells using a plurality of machine learning models, the plurality of machine learning models comprising a respective machine learning model for each gene in the first plurality of genes including a first machine learning model for a first gene in the first plurality of genes, the tumor expression levels including a first tumor expression level for the first gene in the tumor cells, the determining comprising:
generating a first set of features for the first gene, the generating including:
obtaining, using the expression data, an initial expression level estimate of the first gene in the tumor cells of the biological sample and including the initial expression level estimate of the first gene in the first set of features;
including at least some of the first total expression levels in the first set of features; and
including at least some of the second total expression levels in the first set of features;
providing the first set of features as input to the first machine learning model to obtain an output indicative of a TME expression level estimate of the first gene in the TME cells; and
determining the first tumor expression level for the first gene in the tumor cells using the output of the first machine learning model and a total expression level, in the first total expression levels, for the first gene; and
outputting the tumor expression levels of the first plurality of genes in the tumor cells.

29. At least one non-transitory computer-readable storage medium storing processor executable instructions that, when executed by at least one processor, cause the at least one processor to perform a method for using machine learning to estimate tumor expression levels of genes in tumor cells in a biological sample of a subject having cancer, the biological sample comprising the tumor cells and tumor microenvironment (TME) cells, the method comprising:
obtaining expression data for a set of genes, the set of genes comprising a first plurality of genes associated with the tumor cells and a second plurality of genes associated with the TME cells, the expression data comprising first total expression levels for genes in the first plurality of genes and second total expression levels for genes in the second plurality of genes;
determining the tumor expression levels of the first plurality of genes in the tumor cells using a plurality of machine learning models, the plurality of machine learning models comprising a respective machine learning model for each gene in the first plurality of genes including a first machine learning model for a first gene in the first plurality of genes, the tumor expression levels including a first tumor expression level for the first gene in the tumor cells, the determining comprising:
generating a first set of features for the first gene, the generating including:
obtaining, using the expression data, an initial expression level estimate of the first gene in the tumor cells of the biological sample and including the initial expression level estimate of the first gene in the first set of features;
including at least some of the first total expression levels in the first set of features; and
including at least some of the second total expression levels in the first set of features;
providing the first set of features as input to the first machine learning model to obtain an output indicative of a TME expression level estimate of the first gene in the TME cells; and
determining the first tumor expression level for the first gene in the tumor cells using the output of the first machine learning model and a total expression level, in the first total expression levels, for the first gene; and
outputting the tumor expression levels of the first plurality of genes in the tumor cells.
